(19) United States
(12) Patent Application Publication
(10) Pub. No.: US 2006/0084141 A1
(43) Pub. Date: Apr. 20, 2006

(54) Biosynthetic gene cluster for the maytansinoid antitumor agent ansamitocin

(76) Inventors: Heinz G. Floss, Bellevue, WA (US); Tin-Wein Yu, Baton Rouge, LA (US); Eckhard Leistner, Meckenheim (DE)

Correspondence Address: Medlen & Carroll, 101 Howard Street, Suite 350, San Francisco, CA, 94105, US

(21) Appl. No.: 10/496,351

(22) PCT Filed: Nov. 21, 2002
(86) PCT No.: PCT/US02/37547

§ 371 Date: Aug. 25, 2005

Related U.S. Application Data
(60) Provisional application No. 60/332,158, filed on Nov. 21, 2001.

(51) Int. Cl. C12P 17/18; C07H 21/04; C12P 21/06; C12N 9/10; C12N 1/21; C12N 15/74

(52) U.S. Cl. 435/69.1; 536/23.2; 435/193; 435/320.1; 435/252.35; 435/119; 540/462

(57) ABSTRACT

The present invention relates to compositions and methods involving antitumor agents derived from bacteria. In particular, the present invention provides the ansamitocin biosynthetic gene cluster for the production of novel maytansinoid analogs with potent antitumor activity and reduced human toxicity.

This invention was made in part with Government support by the National Institute of Health Grant Number AI76461. Accordingly, the Government has certain rights in the invention.

### FIELD OF THE INVENTION

The present invention relates to compositions and methods involving antitumor agents derived from bacteria. In particular, the present invention provides the ansamitocin biosynthetic gene cluster for the production of novel maytansinoid analogs with potent antitumor activity and reduced human toxicity.

### BACKGROUND OF THE INVENTION

Maytansinoids are extraordinarily potent antitumor agents, that were originally isolated from members of the higher plant families Celastraceae, Rhamnaceae and Euphorbiaceae, as well as some species of mosses (Kupchan et al., J. Am. Chem. Soc. 94:1354-1356 [1972]; Wani et al., J. Chem. Soc. Chem. Commun. 390: [1973]; Powell et al., J. Nat. Prod. 46:660-666 [1983]; Sakai et al., J. Nat. Prod 51:845-850 [1988]; and Suwanborirux et al., Experientia 46:117-120 [1990]). As shown in FIG. 1, maytansinoids are 19-membered macrocyclic lactams related to the ansamycin antibiotics of microbial origin, such as rifamycin B and geldanamycin (Rinehart and Shield, Fortschr. Chem. Org. Naturst. 33:231-307 [1976]). The similarity between these compounds stimulated a search for maytansinoid-producing microorganisms, leading to the isolation of the ansamitocins from the Actinomycete Actinosynnema pretiosum ssp. pretiosum and a mutant strain A. pretiosum ssp. auranticum (Higashide et al., Nature 270:721-722 [1977]; and Asai et al., Tetrahedron 35:1079-1085 [1978]). Both the structures and antitumor activity of the ansamitocins were found to be similar to maytansine and other maytansinoids obtained from plant sources.

Mode of action studies have shown that maytansinoids bind to tubulin at a site overlapping the Vinca alkaloid binding site and prevent the polymerization of tubulin (Remillard et al., Science 189:1002-1005 [1975]; Mandelbaum-Shavit et al., Biochem. Biophys. Res. Commun. 72:47-54 [1976]; and Hamel, Pharmac. Ther. 55:31-51 [1992]). Maytansine was found to inhibit the growth of various murine leukemia cell lines, as well as human carcinoma and leukemia cell lines at remarkably low doses (e.g., 10⁻³ to 10⁻⁷ μg/ml) in vitro (Issell and Crooke, Cancer Treatment Reviews 5:199-207 [1978]. Even so, phase I and phase II clinical trials in human cancer patients were disappointing. In phase I trials, dose limiting gastrointestinal toxicity and neurotoxicity were observed, with modest levels of mylosuppression (Komoda and Kishi, in Douros and Cassady (eds.) Anticancer Agents Based on Natural Product Models (Academic Press, NY) pp. 353-389 [1980]; and Issell and Crooke, Cancer Treatment Reviews 5:199-207 [1978]). A few partial responses were observed in these early trials, leading to a substantial number of phase II trials in patients with different types of tumors (Reider and Roland, in Brossi (ed.) The Alkaloids (Academic Press, NY) vol 23, pp. 71-156 [1984]; Thigpen et al., Am. J. Clin. Oncol. (CCT) 6:273-275 [1985]; Thigpen et al., Am. J. Clin. Oncol. (CC7) 6:427-430 [1985]; Kalser et al., Cancer Treatment Rep. 69:417-420 [1985]; and Ravry et al., Am. J. Clin. Oncol. (CCT) 8:148-150 [1985]). Insignificant response rates were consistently seen in these phase II trials, which lead to the conclusion that further clinical evaluation of maytansine was not warranted.

Nonetheless, the high intrinsic potency of the maytansinoids points to the value of these compounds in cancer therapy. In particular, there remains a need in the art to produce safe and effective new maytansinoid analogs.

### SUMMARY OF THE INVENTION

The present invention relates to compositions and methods involving antitumor agents derived from bacteria. In particular, the present invention provides the ansamitocin biosynthetic gene cluster for the production of novel maytansinoid analogs with potent antitumor activity and reduced human toxicity.

The present invention provides isolated nucleic acids of at least 12 nucleotides in length that specifically hybridize under stringent conditions to the ansamitocin gene cluster I of Actinosynnema pretiosum. In some embodiments, the nucleic acid specifically hybridizes to the sequence set forth in SEQ ID NO:56, or to the complement thereof. In preferred embodiments, the nucleic acid sequence specifically hybridizes to a sequence encoding a protein with ansamitocin biosynthetic activity. In a subset of these embodiments, the ansamitocin biosynthetic activity is a polyketide synthase activity selected from the group consisting of β-ketoacyl-ACP synthase activity, acyltransferase activity, ACP activity, carboxylic acid:ACP ligase activity, β-hydroxyacyl-thioester dehydratase activity, β-ketoacyl-ACP reductase activity and enoyl reductase activity. In other embodiments, the ansamitocin biosynthetic activity is an ansamitocin modifying activity selected from the group consisting of methytransferase activity, amide synthase activity, oxygenase activity, halogenase activity, acylCoA dehydrogenase activity, D-alanyl carrier protein activity, CO dehydrogenase activity, O-methyltransferase activity, 3-O-methyltransferase activity, 3-O-acyltransferase activity, O-carbamoyltransferase activity, kinase activity, ADHQ dehydratase activity, and AHBA synthase activity.

Also provided by the present invention are isolated nucleic acids that comprise a sequence selected from the group consisting of at least one open reading frame from the ansamitocin gene cluster I of Actinosynnema pretiosum, a gene encoding a biologically active portion of the protein produced from the open reading frame, and a gene encoding a biologically active variant of the protein produced from the open reading frame. In some embodiments, the nucleic acid comprises at least one open reading frame of SEQ ID NO:56. In preferred embodiments, the nucleic acid encodes a protein with ansamitocin biosynthetic activity. In a subset of these embodiments, the ansamitocin biosynthetic activity is a polyketide synthase activity selected from the group consisting of β-ketoacyl-ACP synthase activity, acyltransferase activity, ACP activity, carboxylic acid:ACP ligase activity, β-hydroxyacyl-thioesterdehydratase activity, β-ketoacyl-ACP reductase activity and enoyl reductase activity. In other embodiments, the ansamitocin biosynthetic activity is an ansamitocin modifying activity selected from the group consisting of methytransferase activity, amide synthase activity, oxygenase activity, halogenase activity, acylCoA dehydrogenase activity, D-alanyl carrier protein activity, CO dehydrogenase activity, O-methyltransferase activity, 3-O-methyltransferase activity, 3-O-acyltransferase activity, O-carbamoyltransferase activity, kinase activity, aDHQ dehydratase activity, and AHBA synthase activity. Also provided are vectors comprising isolated nucleic acids that comprise a sequence selected from the group consisting of at least one open reading frame from the ansamitocin gene cluster I of Actinosynnema pretiosum, a gene encoding a biologically active portion of the protein produced from the open reading frame, and a gene encoding a biologically active variant of the protein produced from the open reading frame. In some preferred embodiments, the vector further comprises a promoter and a regulator operatively linked to the nucleic acid. In particularly preferred embodiments, the promoter is the pactIII-pactI promoter and the regulator is the actII-ORF4 regulator. Also provided are host cells transformed with the vectors. In some preferred embodiments, the transformed host cell is a bacterial cell selected from the group consisting of a Streptomyces coelicolor cell, a Streptomyces lividans cell, and a Actinosynnema pretiosum cell.

Additionally, the present invention provides transgenic Actinosynnema pretiosum cells having a genome that comprises a disruption of at least one endogenous gene in the ansamitocin gene cluster, wherein the disruption is selected from the group consisting of an insertion, a deletion and a substitution. In some preferred embodiments, the at least one endogenous gene comprises a gene selected from the group consisting of asmB, asm19, asm15, and asm12. In other embodiments, the at least one endogenous gene comprises a gene selected from the group consisting of a gene of the ansamitocin gene cluster II and a gene between the two ansamitocin gene clusters.

Moreover, the present invention provides maytansinoids produced by a bacterial host cell transformed with an expression vector comprising a sequence selected from the group consisting of at least one open reading frame from the ansamitocin gene cluster I of Actinosynnema pretiosum, a gene encoding a biologically active portion of the protein produced from the open reading frame, and a gene encoding a biologically active variant of the protein produced from the open reading frame. In some preferred embodiments, the bacterial host cell is selected from the group consisting of a Streptomyces coelicolor cell, a Streptomyces lividans cell, and a Actinosynnema pretiosum cell. Also provided are embodiments in which the expression vector comprises asmA, asmB, asmC, asmD, and asm09 open reading frames.

Furthermore the present invention provides maytansinoids produced by a transgenic Actinosynnema pretiosum cell having a genome that comprises a disruption of at least one endogenous gene in the ansamitocin gene cluster, wherein the disruption is selected from the group consisting of an insertion, a deletion and a substitution. In some embodiments, the transgenic Actinosynnema pretiosum cell further comprises at least one heterologous biosynthetic gene. In some preferred embodiments, the heterologous biosynthetic gene is derived from a member of the group consisting of Amycolatopsis mediterranei and Streptomyces achromogenes var rubradiris. In a subset of these embodiments, the heterologous biosynthetic gene is derived from a member of the group consisting of the rifamycin biosynthetic gene cluster and the rubradirin gene cluster.

### DESCRIPTION OF THE FIGURES

The following Figures, which form part of the specification, are included to further demonstrate certain aspects and embodiments of the present invention. The invention may be better understood by reference to one or more of these Figures in combination with the detailed description of specific embodiments presented herein. However, it is not intended that the present invention be limited to the specific embodiments presented in these figures.

FIG. 1 shows the structure of the maytansinoids, maytansine and ansamitocin, as well as the structurally related ansamycin antibiotics, geldanamycin and rifamycin.

FIG. 2 depicts the organization of the ansamitocin biosynthetic gene cluster. Panel A provides a restriction map of the genomic region and overlapping inserts of 14 cosmid clones used for mapping and sequencing the cluster. BglII (B 1-4), EcoRI (E1-4) and SacI (S1-57) restriction sites are indicated on the map. The regions D1, D2 and D3 were deleted in the mutants HGF056, HGF057 and HGF051, respectively. Panel B indicates the direction of transcription and the relative sizes of the ORFs deduced from analysis of the nucleotide sequence in clusters I and II. Letters or numbers above the arrows correspond to the ORFs and asm gene products listed in Tables 1 and 2.

FIG. 3 shows the disruption of the ansamitocin asmB locus. Panel A provides a KpnI (K1-5) restriction map of the A. pretiosum ssp. auranticum genome encompassing the C-terminal end of asmA and the whole ORFs of the asmB and asm25 genes. Panel B provides a Southern blot verifying the deletion of the 7.2 Kb DNA fragment (e.g., region D3) of asmB in A. pretiosum HGF051. The 2.5 and 3.5 Kb DNA fragments indicated by rectangles in Panel A were used as a ³²P-dCTP-labeled probe for the blot shown in Panel B.

FIG. 4 depicts accumulation of an N-acetyl triketide. An attempt to co-express the asmA of pHGF7545 and seven AHBA biosynthesis genes (rifGH, rifK-N and rifJ) of pHGF7612 under the control of the pactIII-pactI promoters and the actII-ORF4 regulator failed to result in the expected triketide product. In contrast, co-expression of the asmAB gene of pHGF7547 and the seven rif genes of pHGF7612 gave a triketide derivative.

FIG. 5 depicts the domain organization of the asm PKS and a biosynthetic model for ansamitocin P-3 formation. Each module incorporates the essential KS, AT and ACP domains, while all but one include optional modifying activities (KR, DH, ER). The abbreviations are as follows: ADE, adenylation or carboxylic acid:ACP ligase; ACP, acyl carrier protein; KS, β-ketoacyl-ACP synthase; AT, acyltransferase; DH, β-hydroxyacyl-thioester dehydratase; KR, β-ketoacyl-ACP reductase; ER, enoyl reductase. The putative intermediates in chain extension cycles and the asm genes involved in the various biosynthetic steps are indicated.

### DESCRIPTION OF THE INVENTION

The potent antitumor activity of the maytansinoids stimulated several chemical total syntheses (Smith and Powell, in Pelletier (ed.) Alkaloids, (John Wiley and Sons, NY) vol 2, pp. 149-204 [1984]; Reider and Roland, in Brossi (ed.) The Alkaloids, (Academic Press, NY) vol 23, pp. 71-156 [1984]; and Komoda and Kishi, in Douros and Cassady (eds.) Anticancer Agents Based on Natural Product Models, (Academic Press, NY) pp. 353-358 [1980]), but work aimed at defining structure-activity relationships relied entirely on the natural products and semisynthesis. Interestingly, a number of structural variations are encountered naturally, including different ester side chains at C-3 and presence or absence of the 4,5-epoxide, the N-methyl group, the halogen, and oxygens at C-15 and at the C-14 methyl group (Smith and Powell, in Pelletier (ed.) Alkaloids (John Wiley and Son, NY) vol 2, pp. 149-204 [1984]). Although considerable variation in its structure can be tolerated, the ester group at C-3 was found to be essential for antileukemic activity (Kupchan et al., J. Med. Chem. 21:31-37 [1978]). The free hydroxyl group at C-9 is also essential since an ether derivative has very low activity. The configuration at C-10 and a hydroxy or acyloxy function at C-15 have little effect, but the epoxide function seems to modify the activity. The Takeda Company introduced further modifications by chemical or microbial transformation of ansamitocin P-3, generating a large number of maytansinoid analogs (Smith and Powell, in Pelletier (ed.) Alkloids, (John Wiley and Sons, NY) vol 2, pp. 149-204 [1984]; Reider and Roland, in Brossi (ed.) The Alkaloids, (Academic Press, NY) vol 23, pp. 71-156 [1984]; Izawa et al., J. Antibiot. 34:1591-1595 [1981]; and Kawai et al., Chem. Pharm. Bull. 32:3341-3351 [1984]). The transformations included N-demethylation, deacylation at C-3, hydroxylation at C-15, O-demethylation at C-20, dechlorination, deoxygenation of the epoxide, as well as the replacement of the 9-hydroxy by a sulhydryl group. However, due to the limitation of natural starting materials, many backbone alterations of the parent molecular structure could not be explored.

During development of the present invention, the cloning, sequencing and characterization of the ansamitocin biosynthetic gene cluster (asm) from A. pretiosum was completed. This work permits a detailed analysis of ansamitocin biosynthesis at the genetic and biochemical level and makes it possible to produce genetically engineered maytansinoid analogs carrying backbone structural modifications that are not easily accessible by chemical means.

Based on the origin of the ansa chain and the chromophore, established by feeding experiments with ¹³C- and ¹⁴C-labeled precursors to fermentations of A. pretiosum (Hatano et al., J. Antibiot. 35:1415-1417 [1982]; and Hatano et al., Agric. Biol. Chem. 49:327-333 [1985]), the biosynthesis of the maytansinoids can be predicted to involve the assembly of the carbon framework on a type I modular polyketide synthase (PKS) from 3-amino-5-hydroxybenzoic acid (AHBA) as the starter unit. Chain extension proceeds by incorporation of three propionate and three acetate units and an unusual hydroxylated 2-carbon extender unit (“glycolate unit”) to give a 19-membered macrocyclic lactam. This initial proansamitocin then undergoes a series of post-PKS modifications, which introduce three methyl groups, a halogen, a carbamoyl group, an epoxy group and an ester side chain.

Identification and Cloning of the Ansamitocin Biosynthetic Gene Cluster

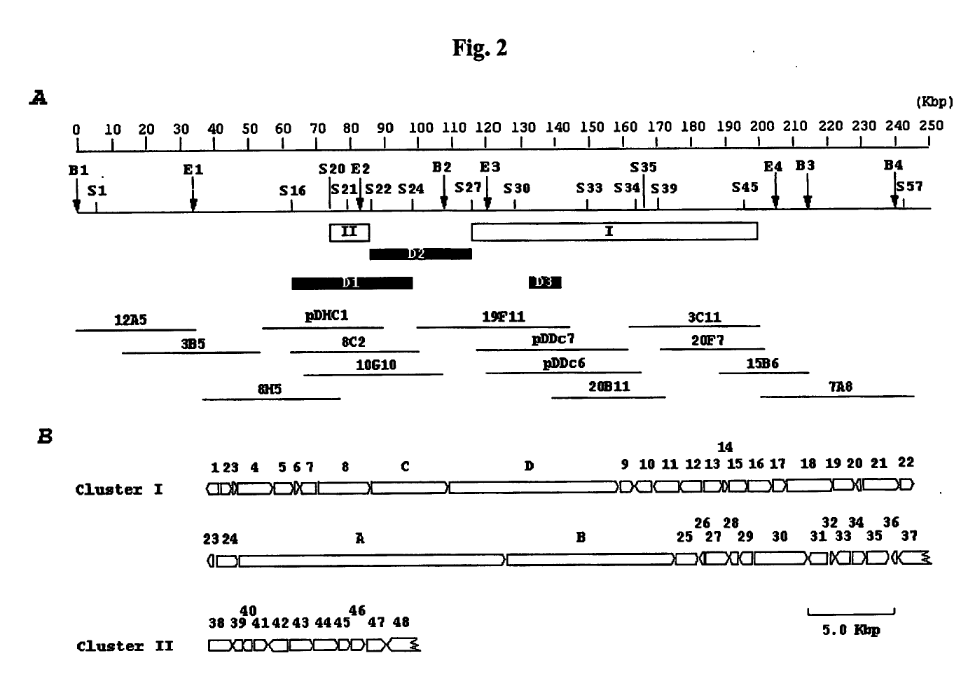

Heterologous hybridization was used to identify genes for AHBA and ansamitocin biosynthesis in A. pretiosum ssp. auranticum ATCC 31565. Initial Southern blot analysis with the rifK gene, which encodes the rifamycin AHBA synthase from Amycolatopsis mediterranei S699 (Kim et al., J. Biol. Chem. 273:6030-6040 [1998]), revealed two separate rifK-homologous DNA fragments in the genome. A total of 250 Kb of contiguous DNA was cloned and mapped with restriction enzymes BglII, EcoRI and SacI (S), and the rifK homologues were located at two regions, S20-22 and S33-34 respectively, as shown in FIG. 2A. Five other AHBA biosynthesis gene homologues from the rifamycin gene cluster (August et al., Chem. & Biol. 5:69-79 [1998]; and Yu et al., J. Biol. Chem. 276:12546-12555 [2001]) were also present and mapped at the same or nearby SacI DNA fragments, which are S20-21 (rifG, -L and -M homologues) and S33-34 (rifJ and -N homologues).

The complete nucleotide sequence of two clusters within the mapped 250 Kb region was then determined, revealing the ansamitocin biosynthetic genes. Cluster I (SEQ ID NO:56) and cluster II (SEQ ID NO:57; GenBank Accession No. U33059) together were shown to contain 50 complete and two partial open reading frames (ORFs) that span 96 Kb (See, FIGS. 2A and 2B). The sequenced genes and the deduced functions of their products are listed in Tables 1 and 2. Two gene disruptions were carried out as described in Example 3 to address the essential roles of the AHBA gene homologues and to define the boundary of the ansamitocin biosynthetic gene cluster (asm) in A. pretiosum. The mutant strain HGF056, in which a 35.3 Kb SacI DNA fragment (S16-24) carrying the rifK-M and -G homologues had been deleted from the genome (See, FIG. 2A), was no longer able to produce AHBA, ansamitocin P-3 or any ansamitocin-related metabolites. Ansamitocin production, however, could be restored by supplementation of the culture with AHBA (See, Table 3). In contrast, there was no significant effect on ansamitocin production in the mutant HGF057, which carried a 30.2 Kb SacI DNA fragment deletion (S22-27) in the region between the two rifK homologues (See, FIG. 2A). Despite the fact that the second rifK homologue and its associated AHBA synthesis genes (S33-34) are separated by 65 Kb from the S20-21 region, two large contiguous regions spanning S30-35 and S39-45 showed strong signals of hybridization to type I PKS probes. In between the two rifK homologues, a carbamoyl transferase, an acyltransferase and halogenase homologue were detected by random sequencing (Freiberg et al., Nature 387:394-401 [1997]; Hara and Hutchinson, J. Bacteriol. 174:5141-5144 [1992]; and Dairi et al., Biosci. Biotechnol. Biochem. 59:1099-1106 [1995]).

TABLE 1
Deduced Functions of ORFs in the Ansamitocin Gene Cluster I

| Protein | # AA | Similarity | Function [Homologue] | ORF |
|---|---|---|---|---|
| AsmA | 4684 | RifA A. mediterranei | polyketide synthase | SEQ ID NO: 1 |
| AsmB | 3073 | RifB A. mediterranei | polyketide synthase | SEQ ID NO: 2 |
| AsmC | 1589 | RifA A. mediterranei | polyketide synthase | SEQ ID NO: 3 |
| AsmD | 3324 | RifB A. mediterranei | polyketide synthase | SEQ ID NO: 4 |
| Asm01 | 241 | ORF4 S. coelicolor | [quinone oxidoreductase] | SEQ ID NO: 5 |
| Asm02 | 214 | NonG S. griseus | [transcriptional repressor] | SEQ ID NO: 6 |
| Asm03 | 88 | unknown | unknown | SEQ ID NO: 7 |
| Asm04 | 748 | Slr2019 Synechocystis sp. | [ABC transporter] | SEQ ID NO: 8 |
| Asm05 | 416 | PatA Synechocystis sp. | [Na/H antiporter] | SEQ ID NO: 9 |
| Asm06 | 97 | unknown | unknown | SEQ ID NO: 10 |
| Asm07 | 348 | EC 2.1.1.38 S. anulatus | methyltransferase | SEQ ID NO: 11 |
| Asm08 | 1117 | unknown | transcriptional regulator | SEQ ID NO: 12 |
| Asm09 | 259 | RifF A. mediterranei | amide synthase | SEQ ID NO: 13 |
| Asm10 | 294 | TcmP S. glaucescens | methyltransferase | SEQ ID NO: 14 |
| Asm11 | 480 | MtmOII S. argillaceus | oxygenase | SEQ ID NO: 15 |
| Asm12 | 441 | PltA P. fluorescens | halogenase | SEQ ID NO: 16 |
| Asm13 | 341 | EC 1.1.1.157 E. coli | [acylCoA dehydrogenase] | SEQ ID NO: 17 |
| Asm14 | 90 | Dltc S. mutans | [D-alanyl carrier protein] | SEQ ID NO: 18 |
| Asm15 | 357 | I30A.22c S. coelicolor | [acylCoA dehydrogenase] | SEQ ID NO: 19 |
| Asm16 | 437 | EC 1.2.99.2 C. thermoaceticum | [CO dehydrogenase] | SEQ ID NO: 20 |
| Asm17 | 167 | MdmC S. mycarofaciens | O-methyltransferase | SEQ ID NO: 21 |
| Asm18 | 913 | SnoA S. nogalater | [transcriptional activator] | SEQ ID NO: 22 |
| Asm19 | 378 | MdmB S. mycarofaciens | 3-O-acyltransferase | SEQ ID NO: 23 |
| Asm20 | 115 | ORFX S. meliloti | unknown | SEQ ID NO: 24 |
| Asm21 | 668 | NolnO Rhizobium sp. | O-carbamoyltransferase | SEQ ID NO: 25 |
| Asm22 | 251 | MitS S. lavendulae | kinase | SEQ ID NO: 26 |
| Asm23 | 144 | RifJ A. mediterranei | aDHQ dehydratase | SEQ ID NO: 27 |
| Asm24 | 388 | RifK A. mediterranei | AHBA synthase | SEQ ID NO: 28 |
| Asm25 | 402 | OleG2 S. antibioticus | glycosyltransferase | SEQ ID NO: 29 |
| Asm26 | 80 | NcnC S. arenae | [acyl carrier protein] | SEQ ID NO: 30 |
| Asm27 | 341 | YxnA B. subtilis | [glucose 1-dehydrogenase] | SEQ ID NO: 31 |
| Asm28 | 154 | unknown | unknown | SEQ ID NO: 32 |
| Asm29 | 193 | Betl S. meliloti | [transcriptional regulator] | SEQ ID NO: 33 |
| Asm30 | 1005 | YrhJ B. subtilis | cytochrome P450 | SEQ ID NO: 34 |
| Asm31 | 348 | RpoT M. leprae | [sigma factor] | SEQ ID NO: 35 |
| Asm32 | 67 | unknown | unknown | SEQ ID NO: 36 |
| Asm33 | 190 | EC 1.5.1.3 T. maritima | [dihydrofolate reductase] | SEQ ID NO: 37 |
| Asm34 | 204 | SCD95A.38c S. coelicolor | [transcriptional regulator] | SEQ ID NO: 38 |
| Asm35 | 392 | RP698 R. prowazekii | [bicyclomycin resistance] | SEQ ID NO: 39 |
| Asm36 | 105 | unknown | unknown | SEQ ID NO: 40 |
| Asm37 | 558+ | unknown | unknown | SEQ ID NO: 41 |

TABLE 2
Deduced Functions of ORFs in the Ansamitocin Gene Cluster II

| Protein | # AA | Similarity | Function [Homologue] | ORF |
|---|---|---|---|---|
| Asm38 | 426 | LysA M. tuberculosis | [DAP-decarboxylase] | SEQ ID NO: 42 |
| Asm39 | 144 | AbaA-ORFA S. coelicolor | [regulator] | SEQ ID NO: 43 |
| Asm40 | 128 | SCH5.12c S. coelicolor | [sigma factor antagonist] | SEQ ID NO: 44 |
| Asm41 | 190 | SCJ1.02c S. coelicolor | glycosyl hydrolase | SEQ ID NO: 45 |
| Asm42 | 336 | EpiH S. epidermidis | [transmembrane protein] | SEQ ID NO: 46 |
| Asm43 | 388 | RifK A. mediterranei | AHBA synthase | SEQ ID NO: 47 |
| Asm44 | 387 | RifL A. mediterranei | oxidoreductase | SEQ ID NO: 48 |
| Asm45 | 212 | RifM A. mediterranei | phosphatase | SEQ ID NO: 49 |
| Asm46 | 266 | unknown | unknown | SEQ ID NO: 50 |
| Asm47 | 342 | RifG A. mediterranei | aDHQ synthase | SEQ ID NO: 51 |
| Asm48 | 518+ | MalT E. coli | [transcriptional activator] | SEQ ID NO: 52 |

TABLE 3
Actinosynnema pretiosum Mutants

| | | Ans P-3 | |
|---|---|---|---|
| Strain | Mutant | Production | Ans Accumulation |
| HGF051 | ΔasmB | no | no |
| HGF052 | Δasm19 | no | N-demethyl-desepoxy- |
| | | | maytanssinol |
| HGF053 | asm15:aac(3)IV | no | 10-demethoxyansamitocin |
| | | | P-3 |
| HGF054 | asm12:aac(3)IV | no | 19-deschloro-ansamitocins |
| HGF056 | Region D1 deletion | no | no |
| HGF057 | Region D2 deletion | yes | yes |

The asm Polyketide Synthase Genes

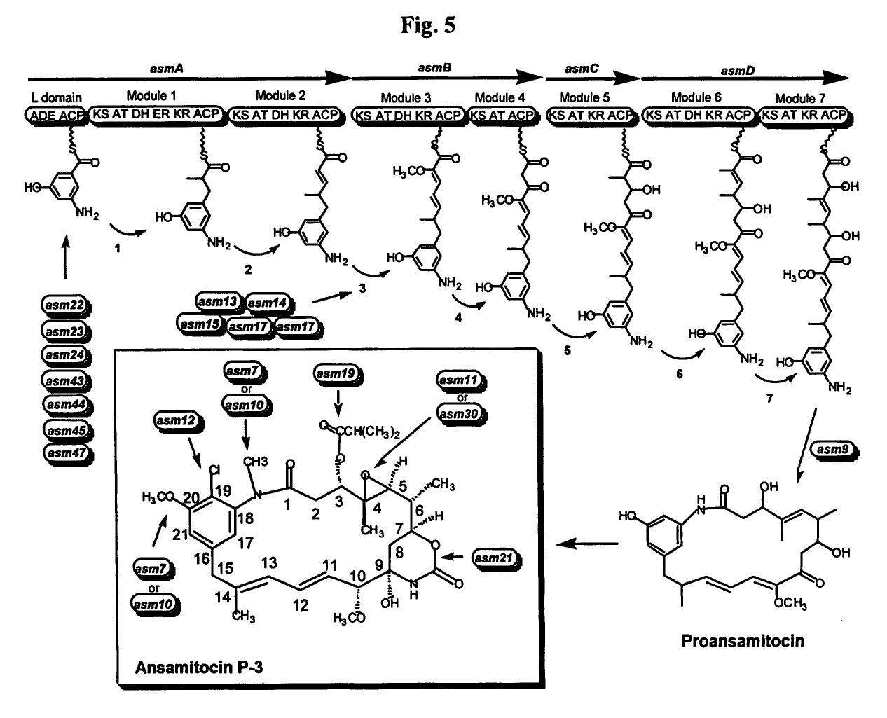

Four large ORFs, asmA-D, encode the loading domain and seven chain extension modules of a multifunctional polyketide synthase (PKS). AsmA (483.1 kDa) contains a chain initiation domain, consisting of an acyl carrier protein (ACP) and an adenyltransferase (Admiraal et al., Biochem. 40:6166-6123 [2001]), presumably required for the activation of the starter unit, AHBA, and the first two expected modules for ansamitocin polyketide chain extension. AsmB (313.2 kDa), AsmC (164.6 kDa) and AsmD (339.3 kDa) contain the next five modules required to continue chain elongation to complete the polyketide portion of ansamitocin. The acyltransferase (AT) domain of module 3 of AsmB, which is predicted to be responsible for the recognition of the unusual hydroxylated 2-carbon extender unit, does not show any unusual sequence signature, which would allow its distinction from other AT domains recognizing malonyl- or methylmalonyl-CoA extender units. Located immediately downstream of asmD is asm9, which encodes a protein with a high degree of sequence similarity to the gene product of rifF from the rifamycin biosynthesis gene cluster and to arylamine N-acetyltransferases, detoxifying enzymes, which acetylate the amino group of aromatic amines (Yu et al., Proc. Natl. Acad. Sci. USA 96:9051-9056 [1999]; and Stratmann et al., Microbiol. 145:3365-3375 [1999]). It is contemplated that the carboxyl terminus of the fully extended polyketide chain is transferred from AsmD to the conserved cysteine residue on the Asm9 protein, and then an amide bond is formed intramolecularly with the amino group of the aromatic moiety to release a 19-membered macrocyclic lactam, proansamitocin (See, FIG. 5).

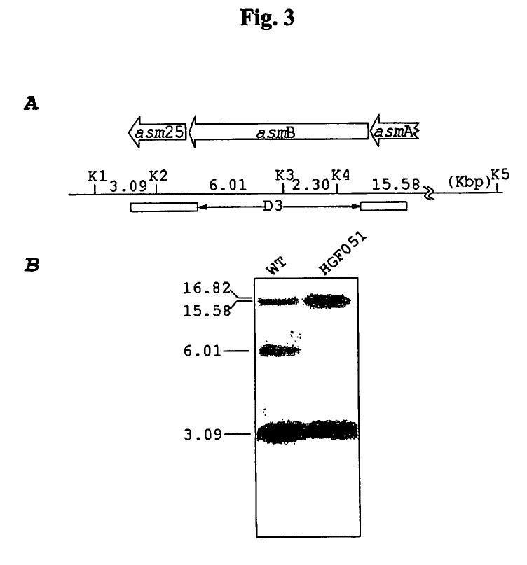

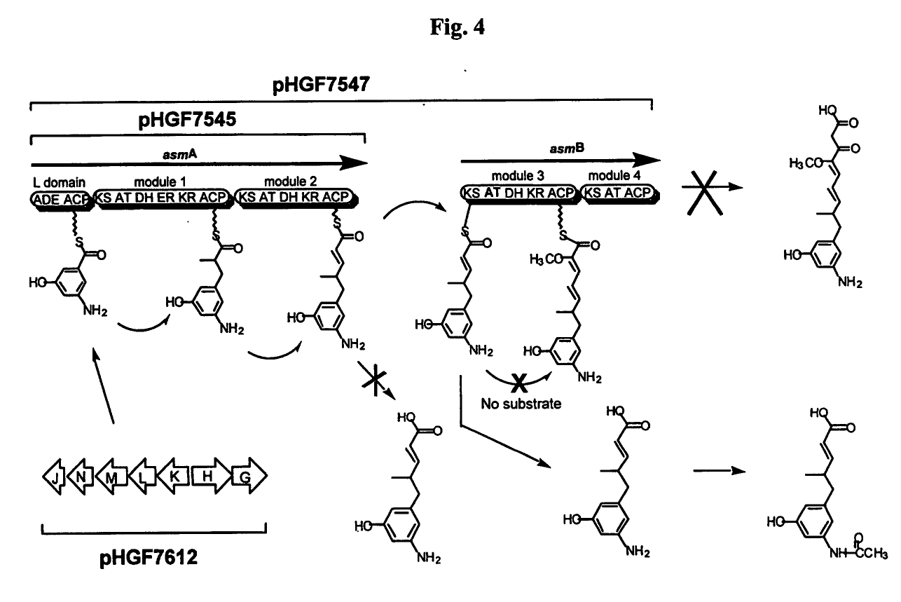

To probe the function of the identified PKS genes in ansamitocin biosynthesis the mutant HGF051 was constructed, in which 2923 amino acid residues were deleted from the coding region of the asmB gene and replaced by five amino acid residues, PSNSI (SEQUENCE ID NO:53; and FIG. 3). The mutant HGF051 failed to produce ansamitocin P-3 or any known related compounds based on a bioassay and HPLC and LC-MS analyses. The functional competence of the cloned PKS genes from the asm biosynthetic gene cluster was further probed by heterologous expression under the control of the bi-directional actI/actIII promoters and the actII-orf4 regulator in S. coelicolor Yu105 (Yu et al., J. Biol. Chem. 276:12546-12555 [2001]). The co-transformants of pHGF7612 (Yu et al., J. Biol. Chem. 276:12546-12555 [2001]), carrying all the genes required for the synthesis of AHBA from the rifcluster, and pHGF7545, carrying asmA, did not accumulate polyketide-related intermediates. However, co-expression of the AHBA synthesis genes with the asmAB cassette, pHGF7547, gave a triketide, as its N-acetyl derivative, with a structure matching that expected for the product assembled by the AsmA protein alone (See, FIG. 4).

The asm AHBA Biosynthesis Genes

Consistent with the Southern hybridization mapping described above, seven AHBA biosynthetic genes that are similar to those cloned from the rifamycin, mitomycin, ansatrienin and naphthomycin biosynthetic gene clusters (August et al., Chem. & Biol. 5:69-79 [1998]; Mao et al., Chem. & Biol. 6:251-263 [1999]; and Chen et al., Eur. J. Biochem. 261:98-107 [1999]) were identified in two groups (See, FIG. 2B). The genes asm22, asm23 and asm24 were found to be in close proximity to asmAB. Asm24 is located upstream of asmA and may share the same transcription unit with the asmAB PKS genes. Its deduced product shows high similarity to the rifamycin AHBA synthase (RifK), which has been demonstrated to catalyze the dehydration of 5-deoxy-5-amino-3-dehydroshikimate (aminoDHS) to AHBA (Kim et al., J. Biol. Chem. 272:6030-6040 [1998]). The asm23 gene, located upstream of asm24 and transcribed in the opposite direction, encodes a protein that has homology to type II dehydroquinate dehydratases of the shikimate pathway and to RifJ, which catalyzes the dehydration of 5-deoxy-5-amino-3-dehydroquinate (aminoDHQ) to aminoDHS in AHBA biosynthesis (Yu et al., J. Biol. Chem. 276:12546-12555 [2001]). Asm22, most similar to MitS and RifN in the mitomycin and rifanycin biosynthesis clusters (August et al., Chem. & Biol. 5:69-79 [1998]; Yu et al., J. Biol. Chem. 276:12546-12555 [2001]; and Mao et al., Chem. & Biol. 6:251-263 [1999]), shows significant similarity to the kanosamine kinase, RifN, of Amycolatopsis mediterranei (Arakawa et al., J. Am. Chem. Soc. 124:10644-10645 [2002]) and the glucose kinase from Streptomyces coelicolor and Bacillus megaterium, which is involved in glucose repression (Angell et al., Mol. Microbiol. 6:2833-2844 [1992]; and Spath et al., J. Bacteriol. 179:7603-7605 [1997]). The genes asm43-47 appear to be organized in a single operon. With the exception that asm46 shows no sequence similarity with any known gene, the deduced proteins of asm43-47 exhibit high homology with the sequences of known products of AHBA biosynthesis genes, including an AHBA synthase (RifK), a class of oxidoreductases that have been implicated in interconversions between hydroxyl and carbonyl groups in sugar moieties (RifL) (Loos et al., FEMS Microbiol. Lett. 107:293-298 [1993]), a phosphoglycolate phosphatase involved in glycolate oxidation (RifM) (Schaferjohann et al., J. Bacteriol. 175:7329-7340 [1993]), and a dehydroquinate synthase (RifG) involved in an early stage of the shikimate biosynthesis pathway, respectively. Interestingly, both isolated AHBA synthase genes were shown to be functionally competent AHBA synthases. Asm43 was expressed in E. coli and AHBA synthase activity was demonstrated, whereas asm24 complemented a rifK mutant of A. mediterranei to restore rifamycin production. Surprisingly, despite extensive sequencing and Southern hybridization analysis, the 3,4-dideoxy-4-amino-D-arabino-heptulosonate 7-phosphate (aminoDAHP) synthase gene corresponding to RifH required in the de novo AHBA biosynthesis, was not found in the cloned 250 Kb DNA region containing the asm cluster.

Genes Involved in the Biosynthesis of the Hydroxylated 2-Carbon Extenter Unit

A set of four contiguous genes, asm13-16, which were identified 4.3 Kb downstream of the asmCD-9 genes, are homologous to genes that encode products linked to the central fermentation pathways for butyrate and butanol production in gram-positive anaerobic bacteria and to the β-oxidation of fatty acids in eukaryotes. The deduced products of asm13 and asm15 show similarities to an NAD-dependent 3-hydroxybutyryl-CoA dehydrogenase and an acyl-CoA dehydrogenase, respectively (Youngleson et al., Gene 78:355-364 [1989]; and Matsubara et al., J. Biol. Chem. 264:16321-16331 [1989]). asm13 and asm14 are potentially translationally coupled. The asm14 product (9.7 kDa) resembles acyl carrier proteins, particularly D-alanyl carrier proteins operating in D-alanyl-lipoteichoic acid synthesis (Boyd et al., J. Bacteriol. 182:6055-6065 [2000]). Asm16 was found to be related to an uncharacterized protein encoded by one of the genes in the fatty acid biosynthetic gene cluster in Bacillus halodurans (Takami et al., Biosci. Biotechnol. Biochem. 63:452-455 [1999]).

A mutant, HGF053, in which the asm15 gene was truncated by inserting an aac(3)IV gene, has been constructed and showed no ansamitocin production, even in the presence of the ester side chain precursor, isobutyrate. However, HPLC, ES-MS and NMR analysis revealed the presence of a small amount of 10-demethoxyansamitocin P-3 in the fermentation. This result rules out the possibility that the asm15 gene product is involved in the formation of the C-3 ester side chain. Instead, it indicates that the asm13-16 subcluster, possibly additionally including asm17, is responsible for generating the unusual hydroxylated polyketide extender unit, which is incorporated at C-9 and C-10 of the ansamitocin skeleton. Asmi 7, apparently transcribed with asm13-16, encodes an O-methyltransferase. Recently, the analysis of the biosynthetic gene cluster for FK-520, another macrolide antibiotic containing hydroxylated 2-carbon extender units, has revealed a set of five genes, fkbGHIJK, resembling asm13-17 (Wu et al., Gene 251:81-90 [2000]). This further supports the notion that asm13-17 are involved in the synthesis of the unusual hydroxylated 2-carbon polyketide chain extender unit.

Genes for the Post-Synthetic Modification of Proansamitocin

The identified asm cluster contains a number of genes that appear to be involved in the further elaboration of the final ansamitocin structure from proansamitocin. The deduced product of asm19 belongs to a family of 3-O-acyltransferases, such as MdmB and AcyA from Streptomyces mycarofaciens and Streptomyces thermotolerans, respectively, which attach short acyl chains in ester linkages to macrolides (Dairi et al., Biosci. Biotechnol. Biochem. 59:1099-1106 [1995]; and Epp et al., Gene 85:293-301 [1989]). The functional assignment as an acyltransferase attaching the 3-O-acyl group of ansamitocin was verified by inactivating asm19 through an internal 549 bp in frame deletion. The mutant, HGF052, produced no ansamitocin P-3, but instead of accumulating the corresponding 3-alcohol, maytansinol, it synthesized a compound identified as N-demethyl-desepoxy-maytansinol.

Asm21 is highly similar to the nodulation proteins of Rhizobium sp. (Freiberg et al., Nature 387:394-401 [1997]) and Bradyrhizobium japonicum (Luka et al., J. Biol. Chem. 268:27053-27059 [1993]), as well as the proteins encoded by cmcH of Streptomyces clavuligerus and novNof Streptomyces spheroides, which carry out the O-carbamoylation steps in cephanycin and novobiocin biosynthesis (Coque et al., Gene 162:21-27 [1995]; and Steffensky et al. Antimicrob. Agents. Chemother. 44:1214-1222 [2000]). On the basis of the analogous chemistry involved, asm21 is assigned to have the putative function of introducing the cyclic carbinolamide group of ansamitocin (Hatano et al., Agric. Biol. Chem. 49:327-333 [1985]).

The asm30 gene product strongly resembles the bifunctional P450-NADPH: P450 reductase, a fatty acid monooxygenase (P450BM-3) from Bacillus megaterium (Ruettinger et al., J. Biol. Chem. 264:10987-10995 [1989]; and Sevrioukova et al., Proc. Natl. Acad. Sci. USA 96:1863-1868 [1999]). Both the heme-P450 and the FMN/FAD-containing reductase domains are linked together on a single 108.6 kDa polypeptide. P450BM-3 performs as a self-sufficient fatty acid hydroxylase, converting lauric, myristic, and palmitic acids to omega-1, omega-2, and omega-3 hydroxy analogs. Analogously, Asm30 is believed to be responsible for the double bond epoxidation occurring at C-4 and C-5 of ansamitocin.

Ansamitocin biosynthesis requires three methylations of the aryl nitrogen and the oxygens at C-10 and C-20, all utilizing S-adenosyl-L-methione (AdoMet) as the methyl donor (Hatano et al., Agric. Biol. Chem. 49:327-333 [1985]). Three AdoMet-dependent methyltransferase genes, asm7, asm10 and asm17, were identified in the asm cluster. The product of asm17 is an O-methyltransferase homologue similar to MdmC from the midecamycin cluster of Streptomyces mycarofaciens (Hara and Hutchinson, J. Bacteriol. 174:5141-5144 [1992]), an O-metyltransferase from the carbomycin cluster of Streptomyces thermotolerans (Epp et al., Gene 85:293-301 [1989]), and the caffeoyl-CoA 3-O-methyltransferases from plants (Martz et al., Plant Mol. Biol. 36:427-437 [1998]). Because of its linkage to asm13-16, asm17 is considered likely to encode the methyltransferase introducing the O-methyl group at C-10. Asm7 is most closely related to a puromycin O-methyltransferase from Streptomyces alboniger (Lacalle et al., Gene 109:55-61 [1991]) and other Streptomyces antibiotic biosynthetic O-methyltransferase (Mao et al., Chem. & Biol. 6:251-263 [1999]; and Madduri et al., J. Bacteriol. 175:3900-3904 [1993]). It may be responsible for either the N-methylation or the aromatic (C-20) O-methylation. Asm10 has significant similarity to proteins of unknown function from Mycobacterium tuberculosis and S. coelicolor, and shares weak similarity with TcmP, an O-metyltransferase involved in tetracenomycin C synthesis in Streptomyces glaucescens (Decker et al., J. Bacteriol. 175:3876-3886 [1993]).

The asm10 gene appears to be polycistronically transcribed with asm11 and asm12. The deduced products of asm11 and asm12 show similarity to a monooxygenase and a halogenase, which are equivalents of Cts8 and Cts4 in Streptomyces aureofaciens, responsible for tetracycline 6-hydroxylation and ring chlorination, respectively (Dairi et al., Biosci. Biotechnol. Biochem. 59:1099-1106 [1995]). The function of Asm11 is unclear, but it may be an alternative candidate to the P450 monooxygenase encoded by asm30 to catalyze the epoxidation at C-4/C-5. The assignment of Asm12 as an aromatic halogenase introducing the chlorine at C-19 was confirmed by inactivation of asm12 to give a mutant, HGF054, which no longer produces ansamitocin P-3. HGF054 accumulates a series of 19-deschloro-ansamitocin derivatives, rather than a single compound, indicating that the presence of the halogen is not an absolute requirement for the occurrence of some of the other post-PKS modification reactions. Regardless, an understanding of the mechanism(s) is not necessary in order to use the present invention.

Thus, the cloning of the asm gene cluster from A. pretiosum and the production of the present invention provides the tools necessary for the genetic engineering of novel maytansinoids for evaluation as potential antitumor drugs with improved efficacy. As discussed herein, the strategy utilized the unique rifamycin biosynthesis genes (e.g., rifK and other AHBA synthesis genes) as probes to identify the asm genes. Surprisingly, two rifK homologues, located 68 Kb apart, were found in A. pretiosum. Sequence analysis of the surrounding DNA revealed that neither homologue is accompanied by a full set of the genes required for AHBA formation (Yu et al, J. Biol. Chem. 276:12546-12555 [2001]). The rifK homologue identified in cluster I is associated with almost the full complement of genes expected for ansamitocin biosynthesis (See, FIG. 5). These include genes for a complete type I polyketide synthase (asmA-D) and its downloading enzyme (asm9), and all the candidate genes for the predicted downstream processing reactions, encoding a halogenase (asm12), a carbamoyltransferase (asm21), three methyltransferases (asm7, -10 and -17), an acyltransferase (asm19) as well as two candidate genes for the epoxidizing enzyme (asm11 and asm30). In addition, it is contemplated that there are several possible regulatory and transport genes, and a glycosyltransferase gene (asm25), which together with asm41 may be part of a glycosylation/deglycosylation excretion/resistance system (Vilches et al., J. Bacteriol. 174:161-165 [1992]), although it remains to be established whether these genes are involved in ansamitocin biosynthesis.

Absent from cluster I are the rifL and rifM homologues, asm44 and asm45, encoding an oxidoreductase and a phosphatase, respectively, which are known to be essential for AHBA formation (Yu et al., J. Biol. Chem. 276:12546-12555 [2001]), as well as an aminoDHQ synthase gene (asm47); these are associated with the second rifK homologue in cluster II. Thus it follows that both clusters I and II must be required for ansamitocin formation, and this was confirmed by deletions of the asmB gene from the cluster I and of a large DNA fragment carrying the cluster II, respectively. Since ansamitocin production can be restored in the cluster II deletion mutant, HGF056, by supplementation with AHBA, cluster II contains genes only required for the formation of the starter unit, AHBA. The 30 Kb of DNA located between the two clusters carry no genes essential for either ansamitocin formation or growth maintenance, since deletion of this region, including parts of asm37 and asm38, in mutant HGF057 caused no discernible phenotypic changes in the organism. It is not clear why the asm genes are split into two separate groups. It can be speculated that they were at one time present as a single cluster and were, in a more recent event, separated by a reorganization of the genome sequence. However, an understanding of the mechanisms(s) is not necessary in order to make and use the present invention.

There are indications that RifK may have two enzymatic activities, the well-characterized dehydratase activity aromatizing aminoDHS (Kim et al., J. Biol. Chem. 272:6030-6040 [1998]) and an aminotransferase activity introducing the nitrogen into a carbohydrate precursor in AHBA formation in Amycolatopsis mediterranei (Yu et al., J. Biol. Chem. 276:12546-12555 [2001]). The presence of two rifK homologues in the asm gene cluster is consistent with this notion. Although both gene products are functionally competent as AHBA synthases, they may be optimized for different reactions in AHBA biosynthesis, one for introducing the nitrogen and the other for the later aromatisation reaction. Interestingly, rifH homologues are absent from the asm gene cluster. The absence of an aminoDAHP synthase gene has also been reported for the mitomycin C biosynthetic gene cluster from Streptomyces lavendulae (Mao et al., Chem. & Biol. 6:251-263 [1999]). Thus, the corresponding early shikimate pathway enzyme, DAHP synthase, may participate in the formation of aminoDAHP and provide an important evolutionary bridge leading to ansamitocin and mitomycin biosynthesis. Indeed, one plant-type DAHP synthase gene has been identified in A. pretiosum and was later shown to be unlinked to the asm cluster.

The formation of the polyketide backbone of the ansamitocins is catalyzed by four genes spanning 38 Kb encoding a type I PKS with an aromatic loading domain closely resembling that of the rifcluster (August et al., Chem. & Biol. 5:69-79 [1998]; and Schupp et al., FEMS Microbiol. Lett. 159:201-207 [1998]) and seven chain extension modules. Each module contains the predicted functional domains for chain extension and chain modification, in an arrangement matching that in other type I PKSs analyzed (Hopwood, Chem. Revs. 97:2465-2497 [1997]), and the modules are arranged colinear with their function in the biosynthetic assembly process (See, FIG. 5). The PKS genes asmAB are separated from asmCD by another set of asm biosynthesis genes, involved in the synthesis of substrates for chain initiation and extension and in downstream processing. The last PKS gene is followed by a gene encoding an amide synthase that is recognized based on work with the rifamycin system as the “downloading” enzyme (Yu et al., Proc. Natl. Acad. Sci. USA 96:9051-9056 [1999]; and Stratmann et al., Microbiology 145:3365-3375 [1999]). It catalyzes the release of the completed polyketide chain from the PKS and its cyclization to a macrocyclic lactam by intramolecular amide bond formation. Therefore, AsmABCD+9 are contemplated to constitute the minimal enzymatic machinery required to catalyze the formation of a hypothetical proansamitocin, the first complete, cyclic polyketide precursor of ansamitocin.

The polyketide synthesized by the asm PKS shows two unusual features. One is the incorporation of a rare hydroxylated 2-carbon extender unit in the third chain extension step. Such “glycolate” units are found in a number of other antibiotics, such as geldanamycin (Haber et al., J. Am. Chem. Soc. 99:3541-3544 [1977]), leucomycin (Omura et al., J. Antibiot. 36:611-613 [1983]), soraphen (Hill et al., J.C.S. Chem. Commun. 2361-2362 [1998]), FK-520 and FK-506 (Byrne et al., Dev. Ind. Microbiol. 32:29-45 [1993]) and concanamycin A (Bindseil and Zeeck, Liebigs Ann. Chem. 305-312 [1994]). Their origin is not entirely clear from the various feeding experiments reported (Hatano Agric. Biol. Chem. 49:327-333 [1985]; Haber J. Am. Chem. Soc. 99:3541-3544 [1977]; Omura et al., J. Antibiot. 36:611-613 [1983]; Hill et al., Chem. Comm. pp. 2361-2362 [1998]; Byrne et al., Dev. Ind. Microbiol. 32:29-45 [1993]; Bindseil and Zeeck, Liebigs Ann. Chem. pp. 305-312 [1994]; and Ono et al., J. Antibiot. 51:1019-1028 [1998]). The C-2 hydroxy group of this extender unit is usually methylated, although there are exceptions to this rule, as in the aflastatins (Ono et al., J. Antibiot. 51:1019-1028 [1998]). A subcluster of five genes, asm13-17, which forms an operon, is evidently responsible for the formation of the substrate for this particular chain extension step, as has been proposed earlier for the corresponding genes in the FK-520 cluster (Wu et al., Gene 251:81-90 [2000]). Evidence supporting this suggestion, and ruling out an alternative function in the formation of the ansamitocin C-3 ester side chain, comes now from the inactivation of asm15, which resulted in the incorporation, albeit inefficiently, of a malonate instead of the hydroxymalonate chain extension unit. One of the genes in this subcluster, asm14, encodes an acyl carrier protein which after activation by transfer of a phosphopantetheinyl group (Lambalot et al., Chem. & Biol. 3:923-936 [1996]), may carry the hydroxymalonate extender unit to the PKS as a thioester. Asm14 is much more similar to ACPs in non-ribosomal peptide synthases (NRPS) than in PKSs, as it encodes a peptidyl-carrier protein which is likely to carry an aminoacyl or a hydroxyacyl group. It is suggested (Wu et al., Gene 251:81-90 [2000]) that the chain extension substrate hydroxymalonyl-ACP is synthesized on this ACP. Since the methyltransferase gene asm17 is part of the operon responsible for these reactions, it is contemplated that the methylation of the hydroxy group is also part of the synthesis, and that the chain extension substrate is methoxymalonyl-ACP.

A second unusual feature of the ansamitocin structure is the position of the double bonds in the ansa ring, located at the C-11 and C-13 positions, rather than at C-10 and C-12 where normal PKS-processing would place them. The unusual placement of the double bonds may be the result of an isomerization, which takes place after the synthesis of the complete polyketide and its release from the PKS, or it may occur on the PKS itself as part of the polyketide assembly process. The modifying domains in modules 2 and 3 of the asm PKS, or for that matter, in any of the modules, show no features that would suggest any unusual catalytic functions. In particular DH3 has the sequence signature of a normal, functionally competent dehydratase domain. On the other hand, there are also no obvious candidate genes outside the PKS region that might encode an enzyme or enzymes, which could catalyze the isomerization of the double bonds as a postsynthetic modification reaction. It is tempting to speculate that the “abnormal” position of the double bonds is related to the presence of the extra oxygen function, hydroxy or methoxy, at C-10 of the polyketide backbone of ansamitocin. However, the formation of 10-demethoxyansamitocin, with the double bonds in the “abnormal” position, in the asm15 mutant rules out such a linkage. Nonetheless, an understanding of the mechanisms(s) is not necessary in order to make and use the present invention.

The precise structure of the first cyclic product of the asm PKS, the hypothetical proansarnitocin (See, FIG. 5), is not certain, as it is dependent upon whether the double bonds migrate before or after polyketide assembly and release, and whether the third chain extension incorporates hydroxymalonate or methoxymalonate. Thus, at least four different structures could plausibly be formed. To examine these possibilities, the expression of the asm PKS genes, together with cassettes of genes ensuring precursor supply, in the heterologous host S. coelicolor is contemplated. The synthesis of an N-acetyl triketide by a transformant expressing an AHBA synthesis cassette, as well as asmAB is a first step in this direction. The curious fact that the accumulation of this product, which should be synthesized by AsmA alone, requires the additional presence of the AsmB protein suggests an interesting facet of the operation of this PKS. Apparently, the product of AsmA cannot be released from the enzyme as long as it is attached to the last ACP domain on the protein (See, FIG. 4). It becomes susceptible to spontaneous release, however, when it can be transferred to the next KS in the assembly line, in the event that the next chain extension reaction cannot take place due to lack of substrate.

The experiments conducted during the development of the present invention set the stage for the metabolic engineering of the ansamitocin biosynthetic pathway to create new maytansinoids containing structural changes which are not accessible by chemical means, with the goal of achieving a better separation of antitumor activity and human toxicity than that associated with the natural products. The present invention also provides means to assess the biosynthetic source of the maytansinoids found in higher plants (e.g., whether the polyketide backbone is synthesized by the plant itself, possibly due to a lateral gene transfer from a microorganism, or whether plants acquire it from an endophytic or otherwise associated microorganism). Regardless, an understanding of the mechanisms(s) is not necessary in order to make and use the present invention.

Definitions

To facilitate understanding of the invention, a number of terms are defined and discussed below.

As used herein, the term “wild type” refers to a gene, gene product or an organism having the characteristics of that gene, gene product or organism when isolated from a naturally occurring source. A wild-type gene is that which is most frequently observed in a population and is thus arbitrarily designated the “normal” or “wild-type” form of the gene.

In contrast, the terms “mutant” and “mutation” refer to a gene, gene product or organism displaying modifications in sequence and or functional properties (i.e., altered characteristics) when compared to the wild-type gene, gene product or organism. It is noted that mutants can be generated by genetic manipulations or can be naturally-occurring mutants that have been isolated and identified by the fact that they have altered characteristics when compared to the wild-type gene, gene product or organism. In preferred embodiments of the present invention, Actinosynnema pretiosum mutants are provided bearing a disruption of an endogenous gene in the ansamitocin gene cluster, where the disruption of the gene is accomplished by insertion, deletion or substitution of one or more nucleotides.

The term “gene” refers to a nucleic acid (e.g., DNA) sequence that comprises coding sequences necessary for the production of a polypeptide or precursor (e.g., asmA, asmB, asmC, asmD, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In particular, the term “asm gene” refers to a full-length asm nucleotide sequence (e.g., contained in SEQ ID NOS: 1-41). However, it is also intended that the term encompass fragments of the asm sequence, as well as other domains within the full-length asm nucleotide sequence. Furthermore, the terms “asm nucleotide sequence” or “asm polynucleotide sequence” encompasses DNA, cDNA, and RNA (e.g., mRNA) sequences.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

The terms “high stringency conditions” and “stringent conditions” when used in reference to nucleic acid hybridization comprise conditions equivalent to binding or hybridization at 42° C. in a solution consisting of 5× SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42° C. when a probe of about 500 nucleotides in length is employed.

“Low stringency conditions” comprise conditions equivalent to binding or hybridization at 42° C. in a solution consisting of 5× SSPE (43.8 g/l NaCl, 6.9 μl NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5× SSPE, 0.1% SDS at 42° C. when a probe of about 500 nucleotides in length is employed.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52 [1989]).

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from 12 nucleotides to the entire nucleotide sequence minus one nucleotide. In some embodiments, the term portion refers to nucleic acid fragments of at least 24 nucleotides in length. In preferred embodiments, the fragments are at least 48 nucleotides in length, in particularly preferred embodiments, the fragments are at least 96 nucleotides in length.

As used herein the term “portion” when in reference to a protein (as in “a portion of a given protein”) refers to fragments of that protein. The fragments may range in size from four consecutive amino acid residues to the entire amino acid sequence minus one amino acid. In some embodiments, the term portion refers to polypeptides of at least 8 amino acids in length. In preferred embodiments, the polypeptides are at least 16 amino acids in length, in particularly preferred embodiments, the polypeptides are at least 32 nucleotides in length.

As used herein, the term “gene cluster” refers to a set of closely linked genes that code for functionally-related products and that are usually grouped together in the genome.

The term “transgenic” refers to any organism into which at least one gene or gene fragment from another species has been introduced or in which an endogenous gene has been specifically inactivated.

“Transgene” refers to a gene inserted by artifice into a cell that becomes part of the genome of the cell, cell line, tissue or organism (i.e., either stably integrated or as a stable extrachromosomal element). The introduced gene can be any nucleic acid (e.g., gene sequence) which is introduced into the genome of an organism by experimental manipulations and may include gene sequences found in that organism so long as the introduced gene does not reside in the same location as does the naturally-occurring gene.

The term “endogenous” refers to a gene or gene product that is native to the biological system, species or genome under study. An “endogenous” gene does not contain nucleic acid elements encoded by sources other than the genome on which it is normally found in nature. In contrast, the terms “exogenous” and “heterologous” refer to a gene or gene product that is foreign to the biological system, species or genome under study.

As used herein, the terms “open reading frame” and “ORF” and “coding sequence” refer to a linear array of codons that encodes an amino acid sequence extending from a translation initiation codon to a termination codon. In bacteria such as Actinosynnema pretiosum, GTG, TTG, ATT, and CTG, as well as the ATG, serve as translation initiation codons.

As used herein, the term “biologically active” refers to a molecule having structural, regulatory and or biochemical functions of a wild type Actinosynnema pretiosum protein encoded by a gene from ansamitocin gene cluster I or II. In some instances, the biologically active molecule is encoded by a homolog of an ansamitocin gene, while in other instances the biologically active molecule is encoded by a portion of an ansamitocin gene. Other biologically active molecules which find use in the compositions and methods of the present invention include but are not limited to proteins encoded by mutant (e.g., variants with at least one deletion, insertion or substitution) ansamitocin genes. Biological activity is determined for example, by restoration (e.g., complementation) or introduction of ansamitocin gene activity in cells which lack such activity, through transfection of the cells with an expression vector containing an ansamitocin gene, derivative thereof, or portion thereof. Methods useful for assessing ansamitocin gene activity include but are not limited to bioassays involving measuring the inhibitory activity of the ansamitocins against Penicillium avellaneum as known in the art (See e.g., Hanka and Barnett, Antimicrob. Agents Chemother. 6:651-652 [1974]). Additional methods useful for assessing ansamitocin gene activity include extraction of the ansamitocins and related compounds from cultures with ethyl acetate, followed by isolation via High Performance Liquid Chromatography (HPLC) and analysis by Electrospray Mass Spectrometry (ES-MS).

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co. [1981]). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “nonconservative substitution” refers to a change in which an amino acid from one family is replaced with an amino acid from another family (e.g. replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.).

As used herein, the term “vector” refers to any nucleic acid molecules that can incorporate foreign DNA and transfer it from one cell to another. Vectors are often derived from plasmids, bacteriophages, or plant or animal viruses. Similarly, the term “expression vector” refers to a recombinant DNA molecule containing a desired coding sequence and appropriate nucleic acid sequences necessary for the expression of the operably linked coding sequence in a particular host organism.

The term “promoter” refers to a DNA nucleotide sequence that when attached to an RNA polymerase molecule, will initiate transcription. The term “regulator” refers to a DNA nucleotide sequence that directly or through its encoded protein influences positively or negatively the transcription of particular coding sequences, their translation into functional proteins or the functions of these proteins.

As used herein, the term “recombinant” refers either to a DNA molecule comprising segments of DNA joined together by means of molecular biological techniques or to a protein encoded by DNA so joined.

The term “transformed host cell” refers to the genetic modification of a cell by incorporation of free DNA. In preferred embodiments, the transformed host cell is a bacterial cell. “Transformation” of bacteria is typically brought about by heat or osmotic shock, electroporation or conjugation with another bacterial species.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “bacteria” and “bacterial” as used herein, refer to prokaryotic organisms (e.g., Archebacteria, Eubacteria, Cyanobacteria). In preferred embodiments, the term “bacteria” refers to Eubacteria, which can be further subdivided on the basis of their staining using Gram stain (e.g., gram-positive and gram-negative).

As used herein, the term “polymerase chain reaction (PCR)” refers to a method for increasing the concentration of a segment of a target sequence in a DNA mixture without cloning or purification (See, e.g., U.S. Pat. Nos. 4,683,195, 4,683,202, and 4,965,188, hereby incorporated by reference). This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” When the template is RNA, a reverse transcription (RT) step is completed prior to the amplification cycles. Thus, this variation is termed “RT-PCR.”

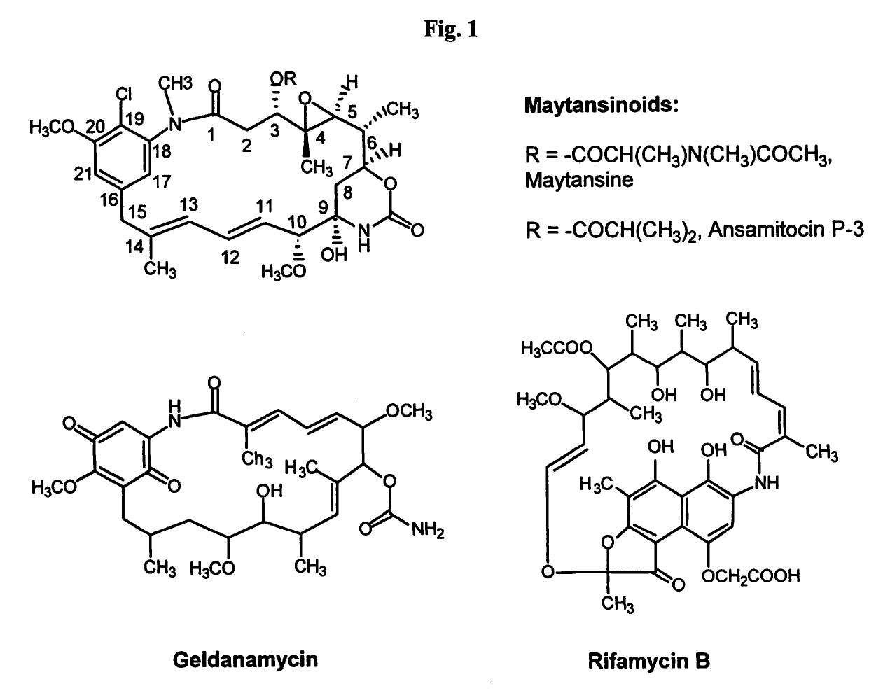

As used herein the term “maytansinoid” refers to compounds resembling maytansine. “Maytansine” is used in reference to the antineoplastic agent isolated from various subtropical trees and shrubs of the genus Maytenus. Maytansine has been assigned the Chemical Abstract Service (CAS) No. 35846-53-8, and the structure of this compound is provided in FIG. 1.

The term “rifamycin” as used herein refers to an antibiotic produced by bacteria of the genus Streptomyces or Amycolatopsis. This group of antibiotics is characterized by a natural ansa structure (chromophoric naphthohydroquinone group spanned by a long aliphatic bridge (O'Neil et al. (eds.) The Merck Index, 13th ed., Merck & Co., Inc., Whitehouse Station, N.J., pp. 1474-1475 [2001]. The term “rifamycin” includes, but is not limited to rifamycins B, O, S, SV and X.

As used herein, the term “rubradirin” refers to an antibiotic produced by Actinomycetes bacteria.

The term “ansamitocin” refers to a maytansinoid produced by the Actinomycete Actinosynnema pretiosum.

As used herein, the term “ansamitocin biosynthetic activity” refers to at least one of the enzymatic activities involved in the building of ansamitocins from more elementary substances. Ansamitocin biosynthetic activity refers to both polyketide synthase activity and ansamitocin modifying activity.

The term “polyketide synthase” as used herein refers to an enzyme or enzymes which catalyze the chain extension of a starter unit, such as 3-amino-5-hydroxybenzoic acid (AHBA), by propionate, acetate, and/or glycolate units. In preferred embodiments, the term “polyketide synthase activity” refers to one or more of the activities catalyzed by the gene products of asmA, asmB, asmC, and/or asmD.

As used herein, the term “ansamitocin modifying activity” refers to at least one of the enzymatic activities involved in the alteration of a nascent (e.g., ansamitocin intermediate) ansamitocin gene product. In preferred embodiments, “anasamitocin modifying activity” includes but is not limited to any one or more of the following activities: methytransferase activity, amide synthase activity, oxygenase activity, halogenase activity, acylCoA dehydrogenase activity, D-alanyl carrier protein activity, CO dehydrogenase activity, O-methyltransferase activity, 3-O-methyltransferase activity, 3-O-acyltransferase activity, O-carbamoyltransferase activity, kinase activity, aDHQ dehydratase activity, and AHBA synthase activity. In particular, the term “anasamitocin modifying activity” refers to one or more of the activities catalyzed by the gene products of asm7, asm9, asm10, asm11, asm12, asm13, asm14, asm15, asm16, asm17, asm19, asm21, asm22, asm23, and/or asm24.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); aa (amino acid); bp (base pair); Kb (kilobase pair); kDa (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); MW (molecular weight); sec (seconds); min(s) (minute/minutes); hr(s) (hour/hours); DMSO (dimethyl sulfoxide); MgCl₂ (magnesium chloride); NaCl (sodium chloride); PAGE (polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); PMSF (phenylmethylsulfonyl fluoride); RT-PCR (reverse transcription PCR); SDS (sodium dodecyl sulfate); Tris (tris(hydroxymethyl)aminomethane); w/v (weight to volume); v/v (volume to volume); YAC (yeast artificial chromosome); HPLC (high performance liquid chromatography); ES-MS (electrospray mass spectrometry); LC-MS (liquid chromatography mass spectrometry); Applied Biosystems (Applied Biosystems, Foster City, Calif.); ATCC (American Type Culture Collection, Rockville, Md.); Difco (Difco Laboratories, Detroit, Mich.); Gene Codes (Gene Codes Corporation, Ann Arbor, Mich.); GIBCO BRL or Gibco BRL (Life Technologies, Inc., Gaithersburg, Md.); Sigma (Sigma Chemical Co., St. Louis, Mo.); and Stratagene (Stratagene Cloning Systems, La Jolla, Calif.).

### Example 1

### Cultivation of A. pretiosum

In this Example, the growth of A. pretiosum is described. Briefly, Actinosynnema pretiosum ssp. auranticum (e.g., ATCC 31565) was obtained from ATCC. For ansamitocin production, the strain was cultivated in YMG medium containing 0.4% yeast extract, 1% malt extract and 0.4% glucose at pH 7.3. The Escherichia coli strain XL1-Blue MRF′ (Stratagene) and ET12567/pUZ8002 (MacNeil et al., Gene 111:61-68 [1992]) were used throughout the study as a cloning host and transient host for conjugation sets, respectively. Conjugations between E. coli and A. pretiosum were performed as known in the art (See e.g., Kieser et al., Practical Streptomyces Genetics, The John Innes Foundation, United Kingdom [2000]). The freshly cultured A. pretiosum mycelia and the overnight grown E. coli cells were mixed and plated on YMG agar plates supplemented with 10 mM MgCl₂. Following incubation at 37° C. for 16 hours, the plates were overlaid with 1 ml of deionized water containing 1 mg of nalidixic acid (Sigma) and 10 μl of a 50 mg/ml DMSO solution of thiostrepton (Sigma). A. pretiosum conjugant colonies were selected by further incubation at 28° C. for 5-7 days. A. pretiosum protoplasts were prepared in P buffer (Kieser et al., Practical Streptomyces Genetics, The John Innes Foundation, United Kingdom [2000]) containing 10 mg/ml lysozyme (Sigma) at 37° C. for one hour. PEG-based transformation (Kieser et al., Practical Streptomyces Genetics, The John Innes Foundation, United Kingdom [2000]) was carried out using denatured plasmid DNA using methods known in the art.

### Example 2

### Cosmid Library Construction and DNA Sequence Analysis

In this Example, the methods used to construct the A. pretiosum cosmid library and to sequence the clones of interest are described. pBluescript SK(−) (Stratagene) was the routine cloning vector used for these experiments. Standard procedures were used to perform plasmid, cosmid and genomic DNA preparations, DNA restriction digests, DNA fragment fractionations, DNA fragment isolations, and ligation reactions (See, e.g., Sambrook and Russell, Molecular Cloning, A Laboratory Manual, 3rd edition (Cold Spring Harbor University Press, NY) [2001]). A. pretiosum ssp. auranticum ATCC 31565 chromosomal DNA was partially digested with Sau3AI, dephosphorylated and ligated into SuperCos 1 (Stratagene), that had been previously digested with XbaI, dephosphorylated and digested with BamHI. The genomic library was made by packaging the ligated mixture with Gigapack III Gold (Stratagene) and transduction into E. coli SURE cells (Stratagene). The entire asm gene cluster was obtained through sequential chromosome walking to extend the primary cosmid clones containing the rifK homologues.

Sequencing templates were obtained by random subcloning 1.5 to 3.0 Kb DNA fragments from selected cosmids generated by ultrasonic fragmentation or by controlled partial HinP1I digestions. Automated DNA sequencing was carried out on double-stranded DNA templates by the dideoxynucleotide chain-termination method with an Applied Biosystems model 373A sequencer. Direct sequencing of cosmid clones using synthesized internal oligonucleotide primers filled occasional sequence gaps. Compilation of the sequence was done using the Sequencher program (Gene Codes). DNA and protein sequence homology searches of various databases were done by using the codon bias of Streptomyces DNA and BLAST programs on the National Center for Biotechnology Information server.

### Example 3

### Asm Gene Inactivations

In this Example, the techniques used to inactivate the Asm gene are described. The knockouts HGF051, HGF052, HGF056 and HGF057 were prepared as follows, with details of the strategy used to create by the D1, D2, and D3 deletions provided herein. A 1.1 Kb DNA fragment of pIJ101 carrying the tsr gene for thiostrepton resistance (Kendall and Cohen, J. Bacteriol. 170:4634-4651 [1988]) and the 0.7 Kb RK2 replication oriT origin (Labigne-Roussel et al., J. Bacteriol. 169:5320-5323 [1987]) were routinely used as the selection marker and for gene-disruption constructs. The target genes or DNA fragments containing the regions to be deleted are shown in FIG. 2A. The D1 deletion (S16-24) was made using the cosmid 8C2 after digestion with SacI, ligation, followed by insertion of the tsr-oriT cassette from pHGF9027 to create pHGF9029. The D2 deletion (S22-27) was made by ligating the 4.6 Kb EcoRI (#2)-SacI (#22) and 5.2 Kb SacI (#27)-EcoRI (#3) DNA fragments to EcoRI-pretreated pDH5 (Hillemann et al., Nucl Acids Res. 19:727-731 [1991]) to create pHGF9011. The D3 deletion, truncating asmB, was made by ligating the 2.5 Kb KpnI-EcoRI and 3.5 Kbp EcoRI-HindIII inserts containing the 5′ and 3′ end sequences of asmB (recovered from a set of pDDc7 subclones), to KpnI-HindIII pretreated pDH5. The oriT fragment was further added to the resulting clone to create pHGF9001. All the constructs, pHGF9001, pHGF9027 and pHGF9029, were delivered into A. pretiosum by either PEG-based transformation or conjugation with E. coli. The thiostrepton-resistant recombinants resulting from the homologous recombination between the delivered DNA vector and the wild-type A. pretiosum were selected, transferred to TSB broth (Difco) for three more rounds of relaxed cultivation, and screened for thiostrepton-sensitive recombinants derived from a second crossover event. Plasmid integrations into the chromosome, as well as the thiostrepton-sensitive recombinants were confirmed by Southern blot analysis of total genomic DNA by well established methods (See, e.g., Ausubel et al., (eds.) Current Protocols in Molecular Biology (John Wiley and Sons, Inc., NY). A representative Southern blot to used to verify the D3 deletion in asmB, is shown in FIG. 3B. Briefly, a 0.7% agarose gel in which 1 μg of KpnI-digested genomic DNA from the wild type and mutant HGF051 strains had been separated, was blotted and hybridized with the ³²P-labeled DNA probe shown in FIG. 3A.

The knockouts HGF053 and HGF054 were prepared by insertion of the aprarnycin gene into asm15 and asm12 respectively. The details of this strategy are provided here for the asm15 knockout. The asm13-17 operon was reconstructed by ligating fragments of the clones A232 (XhoI/ScaI digested) and S125 (EcoRI/ScaI digested) into pBluescript SK(−) previously digested with EcoRI and XhoI. This clone, pHGF9125, was treated with PmlI, and a 1.4 kb fragment of DNA containing the apramycin resistance gene aac(3)IV was blunt-end ligated into this unique site. The resultant clone, pHGF9201, was shown by mapping with PstI to contain the aac(3)IV gene in the same orientation as the disrupted asm15. The 6.23 kb SspI/HindIII fragment containing asm13-17, with the disrupted asm15, was extracted and ligated into pHGF9824, a suicide vector carrying oriT and the thiostrepton resistance gene (tsr), generating pHGF9211-1. This vector was delivered into A. pretiosum by conjugation with E. coli. The recombinants that were sensitive to both thiostrepton and apramycin (resulting from homologous recombination between pHGF9211-1 and A. pretiosum) were grown through 3 further rounds of relaxed cultivation in TSB media. Four hundred single colonies were then screened and 3 isolated that had retained resistance to apramycin, but were sensitive to thiostrepton. Recombinants carrying the disrupted asm15 allele were confirmed by Southern hybridization of their total genomic DNA.

### Example 4

### Heterologous Expression of the asmA and asmAB Genes

In this Example, the methods used to express the A. pretiosum asmA and asmAB genes in S. coelicolor are described. The N-terminal 383 amino acids of the asmA-coding region were PCR-amplified with the sense primer MAY003 PacI, 5′-CAT CGA TTA ATT AAC GGA GAG GCC ATA TGC TGC GAA GCG ACC TGA TCC GTC CC-3′ (SEQ ID NO:54), and antisense primer MAY004 KpnI, 5′-TGC GGT ACC AGC CGT CGC GCA GC-3′ (SEQ ID NO:55) using pDDc7 as a template. The restriction sites in the primers have been underlined. The entire asmA was reassembled by ligating the 1.2 Kb PacI/KpnI-restricted PCR product with the 12.9 Kb KpnI/EcoRI fragment carrying the C-terminal asmA from pDDc7 and cloned downstream of the pactI promoter in PacI/EcoRI-restricted pHGF7505 (Yu et al., J. Biol. Chem. 276:12546-12555 [2001]) to yield pHGF7544. The 15.8 Kb HindIII/EcoRI insert carrying the asmA and actII-orf4 regulatory genes from pHGF7544 was relocated and replaced with the 9.4 Kb HindIII/EcoRI insert carrying the AHBA synthesis genes in the E. coli-Streptomyces bifunctional plasmid pHGF7543, a pHGF7612 (Yu et al., J. Biol. Chem. 276:12546-12555 [2001]) derivative in which the tsr gene has been inactivated by the insertion of an apramycin-resistance aac(3)IV gene (Blondelet-Rouault et al., Gene 190:315-317 [1997]), to yield pHGF7545. Using the same strategy, pHGF7547 was constructed to assemble the entire asmAB by replacing the 12.9 Kb KpnI/EcoRI insert of pHGF7545 with the 22.1 Kb KpnI/EcoRI fragment carrying the C-terminal asmA and asmB from pDDc7.

### Example 5

### Ansamitocin Detection and Analysis

In this Example, the biochemical techniques used to detect ansamitocin production are described. Ansamitocins and related compounds were extracted from the culture with ethyl acetate and analyzed by bioassay or isolated by High Performance Liquid Chromatography (HPLC) and analyzed by Electrospray Mass Spectrometry (ES-MS). The bioassay involved measuring the inhibitory activity of ansamitocins against Penicillium avellaneum as known in the art (See e.g., Hanka and Barnett, Antimicrob. Agents Chemother. 6:651-652 [1974]). The assay was carried out as a standard paper disk assay, in which the diameter of the inhibition zone around a filter paper disk containing the sample on a lawn of the test organism was measured. Liquid Chromatography Mass Spectrometry (LC-MS) analyses were carried out using a Shimadzu LC-10AD pump connected to a Fisons Quattro II mass spectrometer.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in relevant fields, are intended to be within the scope of the present invention.

We claim:

1. An isolated nucleic acid of at least 12 nucleotides in length that specifically hybridizes under stringent conditions to the ansamitocin gene cluster I of Actinosynnema pretiosum.

2. The isolated nucleic acid of claim 1, wherein said nucleic acid specifically hybridizes to the sequence set forth in SEQ ID NO:56, or to the complement thereof.

3. The isolated nucleic acid of claim 1, wherein said nucleic acid sequence specifically hybridizes to a sequence encoding a protein with ansamitocin biosynthetic activity.

4. The isolated nucleic acid of claim 3, wherein said ansamitocin biosynthetic activity is a polyketide synthase activity selected from the group consisting of β-ketoacyl-ACP synthase activity, acyltransferase activity, ACP activity, carboxylic acid:ACP ligase activity, β-hydroxyacyl-thioester dehydratase activity, β-ketoacyl-ACP reductase activity and enoyl reductase activity.

5. The isolated nucleic acid of claim 3, wherein said ansamitocin biosynthetic activity is an ansamitocin modifying activity selected from the group consisting of methytransferase activity, amide synthase activity, oxygenase activity, halogenase activity, acylCoA dehydrogenase activity, D-alanyl carrier protein activity, CO dehydrogenase activity, O-methyltransferase activity, 3-O-methyltransferase activity, 3-O-acyltransferase activity, O-carbamoyltransferase activity, kinase activity, aDHQ dehydratase activity, and AHBA synthase activity.

6. An isolated nucleic acid that comprises a sequence selected from the group consisting of at least one open reading frame from the ansamitocin gene cluster I of Actinosynnema pretiosum, a gene encoding a biologically active portion of the protein produced from said open reading frame, and a gene encoding a biologically active variant of the protein produced from said open reading frame.

7. The isolated nucleic acid of claim 6, wherein said nucleic acid comprises at least one open reading frame of SEQ ID NO:56.

8. The isolated nucleic acid of claim 6, wherein said nucleic acid encodes a protein with ansamitocin biosynthetic activity.

9. The isolated nucleic acid of claim 8, wherein said ansamitocin biosynthetic activity is a polyketide synthase activity selected from the group consisting of β-ketoacyl-ACP synthase activity, acyltransferase activity, ACP activity, carboxylic acid:ACP ligase activity, β-hydroxyacyl-thioester dehydratase activity, β-ketoacyl-ACP reductase activity and enoyl reductase activity.

10. The isolated nucleic acid of claim 8, wherein said ansamitocin biosynthetic activity is an ansamitocin modifying activity selected from the group consisting of methytransferase activity, amide synthase activity, oxygenase activity, halogenase activity, acylCoA dehydrogenase activity, D-alanyl carrier protein activity, CO dehydrogenase activity, O-methyltransferase activity, 3-O-methyltransferase activity, 3-O-acyltransferase activity, O-carbamoyltransferase activity, kinase activity, aDHQ dehydratase activity, and AHBA synthase activity.

11. The vector comprising the nucleic acid of claim 6.

12. The vector of claim 11, further comprising a promoter and a regulator operatively linked to the nucleic acid.

13. The vector of claim 12, wherein said promoter is the pactIII-pactI promoter and said regulator is the actII-ORF4 regulator.

14. A host cell transformed with the vector of claim 13.

15. The transformed host cell of claim 14, wherein said transformed host cell is a bacterial cell selected from the group consisting of a Streptomyces coelicolor cell, a Streptomyces lividans cell, and a Actinosynnema pretiosum cell.

16. A transgenic Actinosynnema pretiosum cell having a genome that comprises a disruption of at least one endogenous gene in the ansamitocin gene cluster, wherein said disruption is selected from the group consisting of an insertion, a deletion and a substitution.

17. The transgenic Actinosynnema pretiosum cell of claim 16, wherein said at least one endogenous gene comprises a gene selected from the group consisting of asmB, asm19, asm15, and asm12.

18. The transgenic Actinosynnema pretiosum cell of claim 16, wherein said at least one endogenous gene comprises a gene selected from the group consisting of a gene of the ansamitocin gene cluster II and a gene between the two ansamitocin gene clusters.

19. A maytansinoid produced by a bacterial host cell transformed with an expression vector comprising a sequence selected from the group consisting of at least one open reading frame from the ansamitocin gene cluster I of Actinosynnema pretiosum, a gene encoding a biologically active portion of the protein produced from said open reading frame, and a gene encoding a biologically active variant of the protein produced from said open reading frame.

20. The maytansinoid of claim 19, wherein said bacterial host cell is selected from the group consisting of a Streptomyces coelicolor cell, a Streptomyces lividans cell, and a Actinosynnema pretiosum cell.

21. The maytansinoid of claim 19, wherein said expression vector comprises asmA, asmB, asmC, asmD, and asm09 open reading frames.

22. A maytansinoid produced by a transgenic Actinosynnema pretiosum cell having a genome that comprises a disruption of at least one endogenous gene in the ansamitocin gene cluster, wherein said disruption is selected from the group consisting of an insertion, a deletion and a substitution.

23. The maytansinoid of claim 22, wherein said transgenic Actinosynnema pretiosum cell further comprises at least one heterologous biosynthetic gene.

24. The maytansinoid of claim 23, wherein said heterologous biosynthetic gene is derived from a member of the group consisting of Amycolatopsis mediterranei and Streptomyces achromogenes var rubradiris.

25. The maytansinoid of claim 24, wherein said heterologous biosynthetic gene is derived from a member of the group consisting of the rifamycin biosynthetic gene cluster and the rubradirin gene cluster.


SEQUENCE LISTING (57 sequences)

SEQ ID NO: 1 - DNA, 14055 bp, Actinosynnema pretiosum
atgctgcgaagcgacctgatccgtcccctgcccgaactgctgcgcgagcacgcggccgagcgcggcgaccgcgtcgccttccgcgacgcgcgccgcgccgtcacctgggtcgacctggagctgcgcacgcgcaggctggccgggcacctggccgcgcgcaggctgctgcccggcgaccgcgccgtgatcctgctgggcaaccgggtggagaccgtcgagtgctacctggcgctggtgcgggcgggcgcggtgggcgtgccgctgaacccggtgtccaccgacgccgagctgggcgccctcctcgacgacagcggcgcccggctcgtcctcaccgacgccgcgcacgccgaccagctcgccgggctccgcgccacccggcccgcgctggaggtcgtgcacgtcgaccgggactacgaggcgctcgcgggcgtcgacgccccgctgcccgcgcacgacgcgctggagctggacgacctggcgtggatgctctacacctccggcaccaccggcgcccccaagggcgtgctgtccacccagcgcaactgcctgtggtcggtggccgcctgctacgcgcccgtgctcggtctgaccccggacgaccgggtgctgtggccgctgccgctgttccacagcctgtcgcacatcgccggggtgctggccaccaccgccgtcggcgccaccacccgcgtggtcgacgggcgctccccgcaggacgtgctggccgcgctgcacgaggagcgctccacggtgctcgcgggcgtgcccacgacctaccaccacctggtgcgcgcggccggtgagcgcgggtttctcgcgcccgacctgcgggtcgggctggtcggcggcgcgatcaccacggccgagctgcgggccgccgtcgagcagcggttcggcgtgccgctcatcgacgcctacggcagcaccgagacgtgcgggtcgatcaccgtgaactggccgaccgggcccagggtcgagggctccagcggcctcccggtgccgggtctggcggtgcggctggtcgacccggacaccggcctggacgcgccgaccggcgcggagggcgaggtgtgggtgcgcgggccgagcgtcatggtcggctaccacaaccggcccgaggcgacggccgccgcgctgcgcgacggctggtaccgcaccggcgacctggcgaccagggacgagtccgggttccacgcggtcaccgggcgcatcgacgacgtcgtggtccgcggcggcgagaaggtgcacccggcggaggtggaggcggtgctgcgggccgtgccgggcgtcgcggacgccgccgtggtgggcaggccgcacgacgtgctgggcgaggtcccggtggcgttcctggtgccgggggaggggttcgacccggcgcaggtgctggcggtgtgccgggagcggctgtcgtaccacaaggtgcccgaggagctgtaccagatcgagtccgtgccgcgcacggcctccgggaagatcacccggagggtgctgctggacacgccggcgcggttgcgggcgagcggtgagcggggggcgctgttccgcaccgactgggtgccgctggccgcagcgcccggtggcgcgaccgcgcccggtgcggtggccgggtctggtgccgcgaccgcgtccagtgctgtgaccggtgccggtgccgcgaccggtcctgcccgtgcgaccacgcccgctgctgcgacagcgcccgcgaccacgcccgccgaggcgtcgaccccggtggcggccgcacggccgggcgggcatccggtggccgtcatggcggagcccaccgccccggctcccggtggcgcgccggtggtgctcgacctgcgcggcgacgacgcgcccgcgctgtccggtcgtcccgacctggtgctcacccgaggtgcggtggccgtggacggcgggccggtgcggctcgggcagctcgccgcgtggaccgccgcctgccgcgccgggatcaccgcggtcgacgccgacgacgacggcgcggtcgacccggtcgggctgctcgccgccggtgagccccggctgctcgtgcgctccggggtcgtgctcgtgccgcgcctgcacgaggtccccggctccgtcgtcgggcgcggcgcgctcgacccccgccgggccgtcgccgtcaccggggcggacagcgggaccgccgcggcgctggcccggcacctgcgcggcgcgcacggcgtgcgcgacctgctgctgatcaccgctccccacgggcgagcccgcgcgggcgcgctgcgcgacgacctcgtggcctcgggcgcgcgggtcgtgctcgccgtcgccggaccggacgcgctcgaccgcgcgctcagccgggcgccgtccccgctgggcgcgctcgtgcacaccggcgacggggacccggaggccctgccccgcctgctcggcgcgcgggtcgagctggtggtgtgcgcgaccgcgttcgactcggccgccgcggaggacgtcgtgctgcgccgccgcgccgccgggatgcccgcgcggtgcctggcctggggaccgtgggaggccgagaccgcgcaggcccggccggggctggccgtgctgccgctgcgggacgggctggccgcgttcgacgccgcgctcggcgccgacgagccggtcgtctcggtgctgcgccccgaccccgacctggtcgccggggacgtgcccgcgccgctgcgcgacctgctgcacgtggtcgccgccgaacccgagccggtccgggtggccgagctgcgggcgagcctggccggtcgcggggagcgggagcgcaggcggcggctgctcgacctggtgcgcgccgagaccgcggccctgcggcccgccgagttcctggtcgacgggcacgagcgcggcttcgccgaactcggcttcacctccgccacctcggtcgccctgcgcaaccggctcaccgccgcgaccgggctcgcgctgcccgcgaccgccgcgttcgagcaccccaccccgaccgcgctcgccgccctgctgcacgacgagctgttcggcgcgtcgcggaccccggtcgcgcccgcgcgcgccgtcgagcgccccgtcgagcgctcgggagagccgatcgcggtcgtcgccgccgggtgcaggctgcccggcggcgtcgcctcccccgaggacctgtgggaggtcgtgcgctccggcgcggacgtggtgtcgccgttcccggtcgaccggggctgggacctggacgcgctgcgcggcgcctgcgcgggcggcgggttcctggccgacgccgccctgttcgacgccgccctgttcggggtgcacccgcgcgaggcactcgcgatggacccgcagcagcggctgctgctcgaagtcgcgtgggaggtgttcgagcgcgccgggatcgacccgcgctcggtggcgggcgacgggaccggcgtgttcgccgggctgatgggccacgactacgggtcgcggccggggctggaccggcagctcgtggagggctacctgagcaccggcaacgcgggcagcgtcgcctccgggcggatcgcctacgcgttcgggctcaccgggcccgcgctcaccgtggacaccgcctgctcgtcgtcgctggtggcggtgcacctggccgtgcagtcgctgcggcgcggcgagtgctcgatggcgctggcgggcggcgcgacggtcatggccacgcccgccgacttcgtcgagttcgcccggcagggcgcgctggccccggacggccgctgcaagccgttctcggcgtcggcggacgggacggcgtggtccgagggcgcggggttggtgctgctggagcggctgtcggacgcgcggcgcaacgggcgtcgggtgctggcggtgatccggggcagcgcggtgaactcggacggggcgtcgaacgggttgacggcgccgagcggtgcggcgcagcagcgggtgatccgggcggcgctcgcggacgccggtctgtccacttcggacgtggacgcggtggaggcgcacgggaccgggacgcggctcggtgatccgatcgaggcccgcgcgctggtcgcgacctacgggcgggaccgggacggcgagccgctgtggctcggctcgctgaagtcgaacttcgggcatccccagggcgcggccgggatcgccgggctcatcaagctcgtgcaggcgctgcggcacggggtgctgccgcgcaccctgcacgtggacgcgcccacccccgaggtcgactgggacggggtggaggtgctggccgagcagcgcgcgtggcccgaggtggaccggccgcgtcgcgcggcggtgtcctcgttcggggtcagcggcaccaacgcgcacgtggtcctggagcaggcgcccgccgagaccgccgcgcccgccgcgcccgagcgcgggcccgtgccgctgccggtgtccgccgtcgacgcggcggcgctgcgcgcccaggcccggcggctggccgacttcctcgccccctccgacgtcacggccgccgagctggcgggcgcgctgagagccggccgggccgacctcggcgagcgcgccgtcgtggtcgccgccgaccgggcgggcgcgctcgccgcgctggaggcgctggccgccgacgagccctcgcccgccctggtcaccgggcgcgcggacgtcaccggccgcacggtgctcgtgttccccggccagggcggccagtgggtgggcatgggcgcccggctgctcgacgagtccccggtgttcgccgagcgggtgggcgagtgcgcggcggccgtcgaccccctggtggacttccgcgtggtcgacgtgctgcgcggcggcgacctgagccgggtggacgtcgtccagccggtcgcgttcgtcgtcatggtcgcgctggcccggctgtggcgctcggtcggcgtcgagcccgacgcggtggtcggccactcgcagggcgagatcgccgccgcctgcgtcgcgggagcgctctcactggccgacgcggcccgcgtggtcgtgctgcgcagccgggccatcgccgccgggctgaatggcggcggcatggcctcgatcccgctgccgcaggcccaggtcgaggagctggtcgcgggcaccggggtgtgcgtcgcggcggtcaacggacccggttcggtggtcgtctccggcgaggtcgacgcgctggtcgcccgcgtggagggcgcccgcaggctgccggtggactacgcctcgcactcgcccgccgtggacgtgctgcgcgagggcctgctcgccgacctcgcgccgatcaccccccgaccggccgccatcccgatgctgtccaccgtcacggggaagtggctggacggcgccgagctcgacgccgactactggttcgccaacctgcgccaccccgtccgcttcgccgacgccaccgcgctcctggtcgccgacgggttccgggcgttcgtggaggtcggcgcccacccggtgctcaccgcgcccgtccaggacctgctcgcgggcgcgggcccctcggtcgccaccggcaccctgcgccgcgacgacggcggcctcgaccggttcctgcgctccgccgccgagctgcacgtgcgcggcgtgcccgtcgacctctccccgctggcgcccccgccggtccgggcgctcgacctgcccacctacccgttccagcgcgcccgctactggctggacgccgaccccaccgccgcctccgcgctgcggcacccgctgctcacctccgccaccgacctcgcgggtggcgaggacctggtgtgccaggcgcggctgagcaccgcgcggcacccgtggctcgccgaccacgcggtgcgcgggtccgccgtcctgcccggcacgtgctgggcggagctggccatccgcgccggcgacgaggtcggcgcgcccgcgctggacgagctgctcgtggaggcaccgctggtgctgcccgagcgcggcgcggtggaggtgcagctcaggatcggcgccccggacgccgccgggcgcaggccgctggaggcccacgcccgcgccgagggcggcgcgtggacccggcacgccagcggctccctcgccgccgaccgctccaccccggtcccgctgaccgagtggccgccgaccggcgcgcgcgaggtcgacctggccgggttctacgacagccgggccgccgagggctacgactacgggcccgcgctgcgcgggctgcgacgggcctggacccgcgacggcgaggtgttcgccgaggtggagctgcccgcaccgctgagcccggacggctggggcctgcacccggccctgttcgacgccgcgctgcacgccaccggcctgctgcccccggcgcgggtcgccgccgacgaggtgctgctgccgttctcctggcgggggaccaggctgcgcgccaccggggcgaccgcgctgcgcgtgcgcctgaccgggaccgccgacgaccacgtgctgcgcgcctacgaccccgagggcgcgccggtcgtggaggtcgacgccctgcggctgcgcccggccccggccaccgcgctcgtcggctcgggcgcgctgttccgcgtcgagtgggtcgacctggacttgccggtgtccacaccggactccacgccgggcgcccccgtgctcgaccggcccgtgctcgacctgcccggcggcgacgcccgcaccctcgtcggcctgaccctggaggccctgcgcgagcacgacgagctgaccgtcgtcacgcgcggcgcggccgaggacccggccgccgcggccgtgtgggggctgctgcgcaccgcccagaccgagcggccgggccgcttcgccgtggtcgacctggacgacgaccccgcctcgcgcgccctgctgcccgccctgcccgccaccggcgaaccgcaggtccgggtgcgcgcgggcagggcgagcgcgccgcgcctggcgcgcacccgcgacgacggcctgccgctgcccgccgccccgacctggcgcctgacctgcgacggcggcacgttcaccgacctggccgcgaccccgagcccggccgcgaaccgcccgctggaacccggcgaggtccgggtgggcgtgcgcgccgcgggcgtcaacttccgggacgccgccgtcgtgctgaacgccgtgcccggccagcgcgggctcggcggcgaggccgcgggcgtggtcctggagaccggccccggcgtcaccggcctcgcggtcggcgaccgggggctcggcacgttcgaccgcgacttcggcgcgttcgggccggtcgcggtcaccgacgcccggctgctcgcgcccgtcccgcccggctgggggttcgcgcgcgccgcctcggtgccggtcgcgttcctcaccgccctctacggcctgcgcgacctcgcccaggtcgggccgggcgaccgcgtcctggtgcacgcgggcgcgggcggcgtcggcggcgcggccgtcaggctggccgcgctgctgggcgcggaggtgttcgccaccgcgtccccggcgaagtggccggtgctgcgcgcgctcggcctggacgacgcgcacctggcctcctcccgcgacaccgggttcgccgagcgcttcccgaacgtcgacgtcgtgctgaactcgctcaccggcgagctggtggacgcctcgctgcggctgctcgcgccgggcgggcggttcctggagctgggcaaggtcgacgtgcgcgacctcgtgcgcgccgacgtgacctaccgggcctacgacctgcgcgacgcgggcccggaccgcaccgccgaactgctcgccgaggtgctggggctgttcgcggcgggccgccttgagccgctgccgctgaccgtccgcgacgtccggcacgcccccgaggcgctgcggcacgtcgcgtccggcgggcacgtcggcaaggtcgtgctcaccgtcccccgcccgctcgaccccgagggcaccgtcctggtccacggcggcggcgcgctcggcgcgctcaccgcccgccacctggtgtccgcgcacggcgcccgccacctgctgatcacctcccggcgcggccccgacgcgcccggcgccgccgagctggccgccgacctcgccgaactcggcgccaccacgcggttcgagcgctgcgacgccgccgaccgcgaccgggtcgccgccctgctggccgacctgggcgggcgcccgctgaccgccgtcgtgcacacggcgggcgcgctcgacgacgccctgctcgccgaccaggacgcggcgcgcgtcgacgcggtcctgcgccccaagctcgacggggcgctgctgctggacgagctgacccgcgacgccgacctggccgcgttcgtgctgttctcctccgccgccgggacgctcggcaacgcgggccaggccaactacgccgccgcgaacgccgcgctcgacgcgctggccgcccgccgccgctccgccgggctgcccgccacctccatcgcctggggccactgggagcacaccagcgccctcaccgccgggctcaccgccgccgacctcgcgcgcggggccaggcacggcgcgggctcgctcaccaccaccgaggcgctggccctgctcgacgccgcgctcgcctcgccggaacccgccgtcctcgccgccgcgctcgacccccgccgcgccgccgaggtcccgccgctgctgcgcgacctcgccggaccgcgccgggcggtcgcggcccaggccgtcgccggcgcggaccggccgctgatcgacgtggtcagggcgcacaccgcgaccgtgctcgggcaccgcgacgcctccgccgtggacgcgaaccgcgcgttcaaggagctgggcgtcgactcgctcacggccgtggagctgcgcaaccggctcgccgccgcgaccgggctcgcgctcgccgccacctcggtgttcgaccacccgaccccggcgctgctcaccgcgcacctgctcgccgagctcgggcacgcccccgccgccgggaccaccgcgccccccgccaccgccgccgtcaccgccgacgcgggggagcccatcgcgatcgtgtcgatggcctgccgcttccccggcgacgtgtcctccccggaggacctgtgggacctcgtcgcgggcggcggggacgcgctcaccccgttccccgccgaccggggctgggacctggcggccctgttcgcccccgacccggaccgcgccgggcgcagccacgcccgcgagggcgggttcctcgccgacgtcgcgggcttcgacgccgacctgttcggcatcagcccccgcgaggccatcggcaccgacccgcagcagcggctgctgctggaggtggcgtgggagctgttcgagcgcgcgggcatcgcccccgactcgctgcgcggcaccgacaccggcgtgttcgcgggcgtcatcgcgcacggctacccggtgctgcccgcccactccgaccaggggctggaggggcaccgggtgaccggggtgtccgggagcgtggtgtccggccgggtcgcctacgccttcgggctgcagggaccggcgatgaccgtggacaccgcctgctcgtcgtcgctggtgggcgtgcacctggcggtgcgggccctgcgcggcggcgagtgctcgctggcgctggcgggcggcgtgacggtgctgagcacgccggacctgttcgtggacttctcccggcagcgcgggctcgccgccgacgggcgctgcaaggcgttcggggcgggcgcggacggcaccgggttcggcgagggcgtcgggctggtgctgctggagcggctgtgggacgcgcgccgcaacgggcgccgggtgctggggctggtgcgcggcagcgcggtgaactccgacggggcctccaacgggctgaccgcgcccagcggccccgcgcaggagcgcgtcatcgggcgggcgctcgcggacgccgggctgtcggcggccgacgtggacctcgtggaggcgcacgggaccgggacccggttgggcgacccgatcgaggcgcgggcgctggtggcgacctacgggcgggaccggggcggggagccggtgtggctcgggtcggtcaagtccaacatcgggcacacccaggccgccgccggggtcgccgggctgatcaaggcggtgcaggcgctgcggcacggggtgctgcccgcgaccctgcacgcggccgagcccacccccgaggtcgagtgggacggggtcgcggtgctggccgagcagcgggactggcccgaggtgggcaggccgcgccgggcggcggtgtcctcgttcggggtcagcggcaccaacgcgcacgtggtgctggagcaggcgcccgaggagttgagccccgaggggcggatccccgaggtgcggatccccgacaggcggacttcagaggggcgggtgcctgaggggcgggcgcccgagggacggacgtccggaggccgggcacccgagggccagacatccgagggccgggcgttcggcggccgggtgcccgaagaccagacatccgaagaccggacatccgagggccaggcgtccatggggcgggcgcctgagggccaggcgcccctgggccaggtgtccgagagccgggcgcccgaccaccgggtgcccgccgggcccgaccgggacgtccccggtcacctgccgctcgtgctgtccgcgcacgacccggccgcgctgcgcgccaccgcggcccgcctgctgccggtcgccgagcgcgtcccgctgcccgatctggcgctgtccctggtcaccggccgcgccgccctgccctgccgggcggtgatccccgcccgcgaccggggtcaggcgctggcggcgctgcgcgacctcgcgtccggcgccaccgcgcccgccgagcgggacggcgggggcgtggcgttcctgctgtccggccagggcagtcagcgggccgggaccgggcggctgctggagcggcggttcccggtgttccgggacgcgctgcgcgaggtgtgcgccctgctggaccggcgcgtcgtcggcggtcccggcgtgcgcgccgcgctggacgaccccgggctgctcgccgacacccgctacgcgcaggccggcctgttcgccgtgcaggtcgcgctggtgcgcctgctcgacgcgctcggcgtccgccccgacctgctcgccgggcactcggtcggcgagatcgccgtcgcgcacgccgccggggtgctgtccctggaggacgcctccaccctcgtggccgcgcgcggcgccctcatgcgcgagctgccccccggcgtgatggtcgcggtccgcgcgggcgaggacgaggcgcgcgccgcgctggtcgacggggtggagctggccgccgtcaacggcccccgcgccaccgtgctcaccggcgacgaggccgccgtcaccgaggtcgccgcccgcctcgggcgcgccacgcgcctgcgggtcgcccacgccttccactcggccgccgtcgacgcggtgctgcccgcgttcgccgacgtgctggcgcgcctggacttccgcccgccgcgcgtcccggtggccaccaccgcgcccggcgcgctcgacgcgcccgagtactggctgggccaggcccgcgagccggtgcgcttcgccgacgccgtcaccgacctgcaccggcggggcgcgaccgcgttcgtcgaggtcggccccggtggtgggctggccgccgccgcgcaggagtgcctggacgggcacgacgtgctctgcgcccccctgctgcaccgcgaccgggacgaggaccactccctgatcaccgcgctcggcgcgctgcacgcgaggggcgcgcgcgtcgacttcgccccgctgctggccgacgcgggcggccggaccaccgacctgcccacctacccgttccagcgccgccgcttctggctcgaccccgccccgcccaccaccgacgccgccccgctggagcacccgctgctcaccggggagctggagatccccggctcggacaccgccgcgttcaccggccgcgccgccaccggcgcccacccgtggctggccgaccaccggctgctcgacgcggtcgtcgtgcccggcacggccctgctggacctggtgcgggcggccggtcgccagctcgggctgcccgaggtcgccgagctggtcaacgaggtcccgctggtgctcggcgacggggcggcgctgctgcgcgtcaccgccgccgcgccgggaccggacgggcggcgggaggtggccgtgcactcccggccggagtcggcagggccgcgctcaccgtggacccggcacgtcaccggccagctcgcccccgagtcggggccgctcgcgttcgacctggcgcagtggccgccgccgggcgcgctgccgctgccggtggccgaccaccgggccgcgatggccgccaccggccacgggtacgggcccgcgttcctggggctgcgcgccgcctggaagctcgacgacgaggtgctggtggaggcggtcctgccggacgaggtcgccgacgacgggcacgccgtgcaccccgcgctgctcgacgcggtgctccaggccatcgcgctcaccggcgtcgaggacggctcgcaggtgcgggtgccgttcagctggcgctccgcgcgcggcgcgggccgggcgggccgcgcggtgcgcgcctccctgcggcccgacggcgacggcggcgtccgggtgcgcgtggcctgcccgcggggccgcccgctgctggagatcgcctcgctggtgtcccggccggtcacccccgaccggctgcggccgggggtgctgctgcgccaggcgtggacggaggtcggcccgccgcccggcgccgccgagcccgtgccgctgcgcgaggtcggcgagcgggtgccgccggtggtgcgggtcgacgccgggcacgtccgcgacctgctgccgctgctccaggaggtggtgaccgagccccgctgggagtcgacgcggctgctcgtgacgacctcgggcgcggtggggccgcgcgccggacgcgccgagggcgccgcggtgtggggcctggtgcggtcggccgccgccgagcacccaggccggttcgtcctggccgacctggacgaccggcccgagtccgccgcgctgctgcccgccctcgccaccggcgcgctgccggaggcggtgctgcgcgcgggacgcgtgctcgtgccggggctgcaccgggtctccgcgcccggcgcaggcggggccgtgctcgaccccggcggcgccgcgctgatcaccggcggttccggcgcgctggccgcgctgctggccgagcacctggtgcgcgcgcacggcgtgcgctgcctggtgctgctcagccgcagcgggcgcggaccggacgtgccgggcgccgacgtgcggcacgtgcgcggcgacgtcgcgaaccgggccgacgtggtcgcggcgctggccgccgtgccgccggaccggccgctgacgctggtggcgcacctggccggggtgctcgacgacggcgccctgaccgcgctgacccccgagcggctggacgcggtgctgcgccccaaggccgagggcgcccggcacctggacgagctgaccagggaccgggacctggcggcgttcgtgctgttctcctcggtcgcgggcgcgctcggccacgccgggcaggccaactacgcggcggccaacgcccacctggacggcctcgcgcacgagcggcacgccctcggcctgcccgccacggcgctggcctgggggccgtggaccgagggcatggcgcgcggtcgcggaacgctctcgccgcaggtcgcgctcggcctgttcgacgcggccctgcgcaccggtctgcccgcgctcgtcccgctcgaccgcgaaccggagcgccgggtccccgccccgcgcgccgcgacccccgccccggccgggccgtcgctggccgaccggctgcgcccgatgccgcccgccgagcggcgcgcccacctgctggagctggtcaccgccgaggcgctgggcgtgctgggccacgacgacggggccgcgctgcccgccgggcaggcgttccgcgacgccgggttcgactcgatcaccgcgatggcgctgcgcaaccggctcgtccggctggccggggtgcggctgcccgcgagcgtggtgttcgaccaccccaccccggccgccaccgccgagcgcctggaggccgagctgttcccggccgaccggctgccggggccccggcacggcgacgacttcgacgacatggccgccgacgagctggtccgccgcgcactcggcggcgagtag

SEQ ID NO: 2 - DNA, 9222 bp, Actinosynnema pretiosum
gtgaccacccgtgagcagctcctggacgcgctgcgcgcctcgctgcgggagaacgagcggctgcgcgccaccgggggcgcggccgagccggtcgcgatcgtcggcgcggcctgccgcttccccggcggggtcacctcgcccgaggagctgtgggacctggtcgtcggcgaggtcgacgcggcgggcccgttcccaggcgaccggggctgggacctggcggacctgttcgaccccgaccccgaccgggtcgggcgctcctacgtgcggcacggggcgttcctgcgcgacatggcctggttcgagccggggttcttcgggatctcgccgcgcgaggccctggtgatcgacccgcagcaccggctgctgctggaggtcgcgtgggaggcgttcgagcgcgcgggcatcgccccggactcggtgcgcgggcgcgacgtcggcgtcttcggcggcgtgctcagccacgaccacggctcgcggttccggcccgtgccgccggagctggagggctacctggggaccggcagctccggcagcgccgcgatcgggcgcgtcgcctacaccctcgggctggaggggcccgcgatcacggtggacaccgcctgctcgtcgtcgctggtcgcgatccacctcgcggtgcgggcgctccagcgcggcgagtgctcgatggcgctggcgggcggggtgacggtgatgtcgacgccggacacgttcgtcgagttctcgcggcagcgcgcgctggcccgcgacgggcgctgcaaggcgttctccgccgacgcggacggcaccgcgtggggcgagggcgcgggtctggtgctgctgcaaccgctctccgccgcccggcgggacgggcggcgggtgttggcggtggtgcgcgggtcggcggtgaactccgacggcgcgtccaacgggctcaccgcgcccagcggtcccgcgcagcagcgggtgatccgggcggcgctggccgacgccgggctgtcggcggcggacgtggacctgctggaggcgcacggcaccgggaccgtgctcggggacccgatcgaggcgcaggcgctgctggagacctacgggcgcggtcgcgcgggcgccgggccgctgtggctcgggtcggtgaagtcgaacatcgggcacacgcagggcgcggcgggcgtggccggggtgctgaaggtggtgcaggcgctgcgcaacggcgtcctgccccccaccctgcacgccgcgacgccgaccaccgaggtcgactgggacgccggggcggtgcgggtgctcgaccgggccctgccttggcgtgggggaggggagccagggagtgcactgggggatgctgctggtggcttggagggtggtgctgcggggggccgaccgggtggtgctgccgggggcgtggcgggtggagtcggtgggggcgccgcggcggtgcggctgaggcggggggcggtgtcggccttcggggccagcggcaccaacgcccacctgatcctcgaagaggcgcccgtggcggaggccgtggcggaggccgtggcacccgccacgaccggctcacctgccgcgcccggcgcggttggcgcaccgggcgtgaccgccgcgcccggaaccttcgccgcgaccgctgccgtgccctctgcgccgctgcccgctgccgcgtccgatgcccccgtgcctgtgcccgtgcccgcccccgtcccggtcgtcgtgtccggggccaccaccgccggtctgcgcgcccaggcggcgcgcctggccgggcacctgcgggagcggcccgcgctcggccccgaggacgtcgcccggccgctgctgctctcccgcgcccagcgcgagcggcgcgccgtcgtggtcgcccgcgaccgggaggcgctgctcggcgggctcgacgccctcgcgggtggtgaggccgggcgccggctcgtgagcggcgcggccgacgtcaccgggcgggtggtcctggtgttccccggtcagggcgcgcactggaccggcgtggccgagcggttgtggcgggaggcgcccgccttcgccgacagcatggcccgctgcgccgacgtgctgcgcgacctcgccgggtgggagctgcgggaggtgctcgtcgacccggtcgcgctggagcgcgtcgacgtgctgcaaccggtctcgttcgcggtcgtcgtgtcgctcgcggcgctctgggcgtcggtcggcgtgcggcccgacgcggtcgtcgggcactcgcagggcgaggtggccgccgcccacgtcgcgggcgcgctcacgctcgccgaggccgcccggatcgtcgtgctccgctccgcgctgatcgcccgcgagctgtccggccgcggcgcgatgctcaccgtcgtcgccgacgtcgagcgggtcaccgcgctgctcgccgggttcgaggggcgggtgtgcgtggcggccgtcaacgggcccgcgtcggtcaccgtctccggcgaggacgcggcggtgcgcgagttcgagcgggtcctgtccgcccggcgggtgctgcggtggcggctgcccggcgtcgacttcgccgggcactccccgcaggtcgacgccctgcgcgcggagctgctggccgcactcggcgacgtcgccgcccgcgaacccgagatcccgctgctgtccaccgtcaccggtcggcccgccacccggctcgacgccgagcactggtaccgcaacctgcgcgagcccgtccggttcgccgacgccgtcaccgccctgctcgaccgggggcaccgggtgttcgtggaggtgagcccgcacccggtgctcaccaccagcgtcgtcgacctcgccgcaccgcaccgcaccgccgtcgtgggcacgctccgccgcgacgagggcgggctcgaccggttcctgctgtccgcgggtgagctgcacgtgcgcggcgtgcccgtcgacctcgcccggcacgcgggcgcgggcacggccgaggtgccgaccacggtcttccagcgcgagcggtactggctggacgcgccccgcccgtccggcgacgcgcgcgccgccgggctcgtgccctcggggcacccgctgctgggcgcgctcgtgccgctcgcggacggcgccgtcctcaccgggcggctctcgctgtcggcccagccgtggctcgccgaccaccgggtgctcggcgcggtcgtcgtgcccggtgtcgcgctggtggagctggcggtgcgcgcgggcgacgaggtcggctgcccggcgctggaggagctggtggtggccgcgccgctggtggtcccggagagcggtgacgtggcggtgcgcgtggtcgtcggcccgcgcggcgcggacgggacgcgccccgtggacgtgctctccggcgaccagcccccggtgctgcacgcgcgcggcgtcctcggcccgccccgcgcgcccgagccggtcgggggccagtggccgcccgcgggcgccgaaccggtcgacctcgccgggctctacccggacttcgccgccaagggcctggagtacgggccgcacttccagcggctgacccggctgtggcggcgcggcgacgaggtgttcggcgaggtcgagctgaccggcgagcagcgcagggccgccgggttcgcgctgcacccggcgctgctggagtgcgcgctgcacgtgagcagcttcaccgggcagcgggtcgcgggggacggcggcgcggtggggctgccgttctccttctcccgcgtcgcgctgcaccgctccggcgtggacgcggtgcgggtgcgggcggcgcgggtcggggacggggtcgtctcgctcgacctcgccgacggcgccggccgcccggtggcgcggatcggcgagctggccttccgacgggcgggcgggatccggtccggtccaccgggggtgctgctggagaccgcgtggcgcccggtggagctgcccgaggccccaccgcccgccgacgtgacgaccgtggtgggggactcggtcgagcgggtgctcgccgccctgcgcgagacgctcgccggggacgggcggctgctcgtgctcaccggcgaccccggcgcgccgatcggcacggcggtcggcggggcggtgtgggggctggtgcgctctgcccagctcgaggagcccggccggatcgtcctggccgacctggacaccgcgcccgagcgggcgggcgcggtgctggaccgggtgctggcctccggtgagccgcaggtcgcgatccgcggcggggccgtgctcgtgccccggttgacgccggtcccggtggtcgccgggcccgccgggctggacccggcgaagacggtggtgctcaccggcgggaccggcgcgctcggcaggctcgtcgcgcgccacctggtcaccgagcacggcgcccgcaggctcctgctgctcgggcgcggcggcccggagcgggcgcgcgacctggtcgccgagctgaccggcctgggcgccgaggtggaggccctcgcctgcgacgtcgccgaccggggcgcgctgtcgaccgcgctcgccgggcgcgccctcaccgccgtcgtgcacctggcgggcgcgctggacgacggcgtcgtgaccgcgctgacccccgagcgcctggccgccgcgttcgcgcccaaggcgctggccgcccggcacctggacgagctgaccagggaccaggacctggccgcgttcgtgctgttctcctccgtcgcctcggtgctcggctcggccggacaggccaactacgcggcggccaacgggttcctggacggcctcgcgcacgagcggcacgcgctcggcctgcccgccacgtccatcgcctgggggctgtgggacgtggacagcgcgctggtgggcgagcgcggcgcggccgaccgggagcggctggccgcgtccgggatgcgcccgctgcccgcgcccgccgcgctcgacctgctcgacgccgcgctggcctgcgggcggcccgccgtcaccgcgctggcgctggcccgcgccgccgaccgggcgcccgccgcgctgggcgaggtggtcgcgtcgcgccgggcgcccgcgcccgccgcgccggtaccagggggcccggccgcgctgctcgcgctggtgcgggccgaggccgccgccgtgctcgccctgcccgtcgcgggcgtcgacgagcggcgggcgttccgcgacctcggggtcgactcgctgaccggggtggagctgcgcaaccggctcgccgaggccaccgggctgcggctgcccgcgacgctggtgttcgaccacccgaccccggtcgagctggccgccgcgctgctggagcgggtgggcgggacctcgggaccggcgggcggcgcgtccgcccccgcgcccgcgtccgcccccggcgagccgatcgcgatcgtcggcatggcctgccgcgcgcccggcggggtcgcctcgcccgaggacctgtgggacctggtcgcgggcggcgtcgacgcggtcggcccgttccccgccgaccggggctgggacctggagcggctggtgcaccccgaccccgaccacgagggctccacctacgtcgaccgggccgcgttcctggacgacccggccgggttcgacgcggacttcttcggcatctccccgcgcgaggccctcgcgatggacccgcagcagcggctgctgctcgaagtcgcgtgggaggcgttcgagcgcgcgggcatcgccccggactcggtgcgggggacggacgtcggcgtgttcgcgggcgtcaccaaccacgactacgaccaggtgctcccgcgcggcgacgacggctaccggatcaccggcgtgtcgggcagcgtcgtgtcggggcgcatcgcctacgcgctcgggctggagggccccgcgctgtcggtggacaccgcctgctcggcctcgctggtggcgatccacctcgcggtgcgggccctgcgcggcggcgagtgctcgatggcgctggcgggcggcgcgacggtcatggccacgcccagggggttcgtggagttcgccaggcagcgcgggctcgccccggacggccgctgcaagccgttctcggcgtcggcggacgggacggcgtggtccgagggtgcggggttggtgctgctggagcggctgtcggacgcgcggcgcaacgggcgtcgggtgctggccgtggtgcggggcagcgcggtgaactcggacggggcgtcgaacgggttgacggcgccgagcggtgcggcgcagcagcgggtgatccgggcggcgctcgcggacgccggtctgtccacttcggacgtggacgcggtggaggcgcacgggaccgggacccggttgggcgacccgatcgaggcccgcgcgctggtcgccacctacgggcgggaccgggacggcgagccgctgtggctggggtccctcaagtccaacctcgggcacatgcagggcgccgccgggatcggcggtgtggtgaagaccgtgcaggcgctgcggcacggggtgctgccgcgcaccctgcacgtggacgcgcccacccccgaggtcgagtgggacggtgtcgcgctgctggccgagcagcgcgcgtggcccgaggtggaccggccgcgccgcgcggcggtctcctcgttcggggtcagcggcaccaacgcgcacgtcgtgctggagcaggcccccgccgagcggaccgcagccgaaaaccccggtgggaacgcagccgggcgcgttggggactttcccggcgtgctgccgctggtgctgtccgcgcggagcccggcggccctgcgcgcccaggccgcgcggctcgccgacgtcctcgacgacctgccgctccgggacgtcgcctccacgctcgcgcgggggcgcgccgcgcacgagcaccgcgccgtcctcgtcgccgccgaccgggacgccctgcgcgccgggctgcgcgcgctggccgggggacgtccgcacgccgccgcccgcaccggggtgcgcgcgcccggcgggctggcgttcgccttctccgggcagggcagccagttcgtcggcatggggcgcggcctgcacgccgcgttccccgcgttcgccgaggtgttcgaccgggtgtgcgcggcgctggacgagcggctgggcgagctcaccgggcgctccgtccgggacgtcgtgctcgacgggcccgccgagctgctggcggagaccgtccacgcccagcccgcgctgttcgccctggaggtcgcgctggcgcggctgctggaggggtggggcgtcgtgcccgacgtcgtgctcgggcactcggtcggcgagctggccgccgcgcacgtcgcgggcgcgctgtcggaggcggacgcctgcgcgctcgtcgcggccagggcgcgggccatgcacggggcccggcgcggcggggcgatgctcgcggtgtccgccaccgaggagcaggtcgcgccgctgctgggcgggggcttggcgctggccgccgtcaacgggccgggcgcggtcgtgctgtccggtgacggggacgcggtcgagcgggccgccgccgagtgcgccgggcgcgggtggcggaccagggcgctgcgggtgagccacgccttccactccgcgcacctggacggggtgctcggtgagctggccgtggccgcgcggcgggtcgaggtccggcccccgcgcgtcccgctgctgtccacgctcaccggcgcgcccgccgacgtcgagctgctcgccgacccggactactgggctcggcaggccagggaggccgtccggttcggcgacggcgtcgcccggctgggcgagctgggcgtgacgcgggtgctggaggtggggcccggcggcgcgctggccgccctggtgcgcgagcaggtcgcgccgggggccgcggtggcgcgcgggctggtcgacggggacgaggtggtcgggctgctctccgggatcggcgacctgttcgcgtccggggccccggtcgactggaccgcgctgctgggcgggccgggggcggtcgtcgacctgcccacctacccgttccagcgcaccaggttctggcccgcggcgcaggcgcccggcgcgtggtgcgaggtccgcctggagcggttgcccgcgcctcggggcggcgggtcggcgaccgtgctggaggtggggccgaccgcctcggccgaggagctggcgagcgtcgccgacgtgctggccgagcacagcggcccgctgctggtggtcaccggggacggcgcggtggccgggctggtgcgctcgctgccggaccgcgcggacgtcgtgctggcggacgtggacgacgacccggcgtcggtggcgctgctgcccgcgctgcacggctgcggcgagcccgagctggtggtgcgcgcgggcgaggtgctcgcgcccaggctgcggcgcgtcaccgcgaccggcgccgccccctcggggccggtgctggtggtcggggaggccgcgcggggcgtggtggcggcctgcggggtgcgcgaaccggtcgtgctgcccgccgactcgggccgggcggagctggtggccgcgctggacgaccaccgcccggactgggtcgtgctcgccgtcgcgccggacgacgcgggcctggcgctggccgcgctgctggacgagctgacccgcgaccggccgcccgcgctgttcgcgctgtgcggaccggcgggcgcgctcggcgacgcggcgcgccccgaggacgcggtggccgccggggtgctggccgcgctcgtgcgccgccgccgcggggacggcctgcccgcgaccctgctgtgcgcgggaccgggcacgccgccgctgagccccgagctgggggtggtcgtgttcgcgcggacgcccgaggtggtgctgcccgagccgaggaccgcccagcccgagccggacgcggtcgacctgaccggcccggaccgggaccgggtgctgctggcgctggtgcgcgaccacgccgccgccgtgctgggccgcgcggacggcccggtggacgggcgggccgcgttccgggacctggggttcgactcgatgacggcggtggagctgcgcgaccgcctggccgccgccaccggcctgaccctgcccgcgaccctgctgttcgaccacccgacggcggccgaggtcgccgccgtgctggcggggctgctcgacggcggcgacccggtgacctcgggcctggaccgcctggaggccgcgctcgccgacccgcccgccgaccccgcgctgcgcgcgtccgccgccgccaggctgcgcgccctgctcgccgccctgtccgacgagggcgacggccgggacggcgctggccaggacggcgctgccgcgctccgggacgcgggcgtggcggagctgttcgagttcatcgacacccggctgggccgcgccgagcgggcctga

SEQ ID NO: 3 - DNA, 4770 bp, Actinosynnema pretiosum
atgaccgacgggacccacgagaagctggtcgactacctccggtgggtcaccgccgacctgcacgagacccggcgcaagctcgccgacctggagtcggcgggccacgagccgatcgcgatcaccggcctggcctgccgcgcccccggcggcgtccgcaccccggaagcgctgtgggagctggtggacggcggcgtcgacgcgatcaccggcttccccaccgaccggggctgggacctggccgggctctacgaccccgacccgaccaggccgggcacctgctacacccgcgagggcgggttcctgcacgacgccgccgagttcgacgccgggttcttcggcatcggcccgcgcgaggccgtcacgatcgacccgcagcaccggatgctcctggagacctcctgggaggcggtcgagcgcgcgggcctcgacccgctgtcgctgcgcggcaccggaaccggcgtcttctccggcgtggtctaccagaactacggcaaccggcggcgcgtcgccgaggagttcgagggccacctggccatcggcagctccggcagcgtcgcctccggccgggtcgcctacgcgctcggcctgcacgggcccgcgatcaccgtggacaccgcctgctcgtcgtcgctggtcgcgatccacctggccgcgcagtcgctgcggcgcggcgagtgcgcgatggcgctggcgggcggcgcgacggtcatgcccctgtcggacgtgttcgtggagttcgcccggcagcgcggactgtccccggacgggcgctgcaaggcgttctccgccgacgccgacggcacggcgtggggtgagggcgtcggcgtggtgctgctggagcggctctcggacgcgcgccgcgccgggaggccggtgctggcggtgatccggggcagcgcggtcaactccgacggcgcgtccaacgggctgaccgcgcccaacggggccgcgcagcagcgcgtcatcgaggcggcgctggaggacgcggggctgcggccgtccgacgtggacctgctggaggcgcacggcaccggcacccgcctcggcgacccgatcgaggcggcggcgctccaggccacctacgggcgcgccaggcccgagggcaggccgctgctgctcggctcgctcaagtccaacatcgggcacgcccagggcgcggccggggtgctgggcctggtcaaggtcgtgcaggcgctgcgccacggcgtcgcgccgcgcaccctgcacgccgaccggctcacccccgaggtcgactggacctccggcgcgctgcgcgtgctgaccgaacccgcgccgtggccgcgcggggagcggccccggcgcgcggcggtgtccgcgttcggcatgagcgggaccaacgcgcacctggtcgtggaggagggcgacgcgccgtccgcgccgggggagcggcgtccggcgggcgcgctgccgctgctgctgtccgcgcgcgggggcgcggcgctgcgcgcggcggcggccgacctggtcgagctgccgggggagccggtggacgtgtgctggtcgctgctcaccggccgcgcccggctggtcgaccgcgcggtggtggtcgccgccgaccgggccgggttcaccgccgggctggaggcgctggccgccgaccggccctcggccgccgcggtgaccgggcgggccgacgtggcgggccgcacggtgctcgtgttccccggccagggcggccagtgggtgggcatgggcgcccggctgctcgacgagtcgccggtgttcgcggcggccgtggcgcgctgcgcggcggccgtggagccgctggtggacttccgcgtggtggacgcgctgcgcggcggcgacctcggccgggtggacgtggtgcagccggtgtcgttcgtgctgatggtggcgctggcgcggctgtggcgctcggtcggcgtcgagccggacgtggtcgtcggccactcgcagggcgagatcgcggcggcctgcgtggccggggcgctctcgctggaggacgcggcgcgggtggtggtgctgcgcagccgggccatcgccgccgaggcgggcggcggcggcatggcctcgatcccgctgccgcgcgcggaggtcgaacgcctgatcaccggcaccggggtgtccgtggcggcggtcaacggacccggatcggtggtcgtctccggcgacgtcgacccgctcgtggcgcgggtggagggcgcgcgcaggctgccggtggactacgcctcgcactcgcccgccgtcgacgtgctgcgcgggcggctgctggccgacctcgcgcgggtcacgccccgaccggccgcgatcccgatgctgtccaccgtcacggggaagtggctggacggcgccgagctggacgccgactactggttcgccaacctgcgcgagccggtccgcttcgccgacgcggtcaccgagctcgccggggcggactgcgcggcgttcgtggaggtcggggcgcacccggtgctcaccgccgcgatccaggacgtcctgggcgagcggaccgccgtggtcaccggcacgctgcgccgcgacgacggcggcctcgaccggttcctgcgctccgccgccgcgctgcacgtgcgcggcgtgcccgtcgactgggcccccgtcttcgagggcctggacgcccggcgcgccgacctgcccacctacccgttccagcgcgcccgccactggctccaggaggccgaacccgacgcccggcaggcgggcggcgacgacccgctgtgggcggcggcgcgcgacgccgacggcctgtgcgccgagctggagctcaccgagcccgaccagcgcgccgcgctgcgcgccgtcctgcccgcgctgaccgcctggcacgagcgcaggagcgagcgggccctgctggactcctggcgctaccggatcacctggcgcaccctgcccgaaccgcccgagtccgccccgcgcggcgactggctcgtcgtcggaccgcaccccgacctgctggccgacctgctgcccggcgccgcgctcgaccccggcgacgccgaccgcgccgccctcgccgacctgctccgcccgcacaccgccgtcgcgggcgtccttgccacgccgggcgccgacgtgctcaccctggtccaggcgctcggcgacgcgggcgccaccgcgccgctgtggtgcgccaccaccggggccgtcgcgatctccccgcacgacggcccgccggaccccgcccaggccgccacctggggactgggccgcgtcgtcgcgctggagcaccccggccgctggggcgggctggtcgacctcgccgaccccgccgacccgcgccagcgccgcgggctcgccgccgtcctcgccggacccggcgtcgacgacggaggtgaggaccaggtcgccatccgcgcgcagggcgtgttcgcgcgcaggctcgtgcgcgccgaaccgcaggccccggccgccccgtggaccccgcgcggcacggtcctgatcaccggcggaaccggcggcgtcgccgcgcacgtggcccgccgcctcgccgccctcggcgccgaccacctcgtcctcaccagccgccgcggcccctcggcccccggcgcgcccgcgctcgccgccgacctggaggcgctgggcgcccgcgtcaccaccgccgcctgcgacgtcgccgaccgcgccgcgctcgccgccctgctgaccggcctggagcgcgcgggcgagcaggtcaccgccgtcgtgcacgcggcgggcgccaacgcccagacccccgtcgccgacaccacccccgaggagcacgcgcgcgtccaggccgccaaggcgctcggcgccgagcacctcgacgagctgctgggcgggcgcccgctcgacgcgttcgtcctgttctcctccaacgcgggcgtgtggggcagcggcggccagtgcgcctacgccgccgccaacgcccgcctcgacgcactggcccagcgccgccgcgcgcacggccgcgtcgccacctccgtcgcctggggcgcctgggacggcggcggcatgtccagcgccaccccggacatcgccgcccagctcgcccaggctggcctgcgcctgatgcccccggacctggcggtgtccgcgctgctcgacgccgtcgcccgcgacgagagcaccaccgtcgtgaccgacgtgcgctgggaggcgttcgccgcccggttcaccgccctgcgccgcagccccctgctcgccgacctgcccgaggcccgccccgaaccggtcgccgccaccgccgagcccgaccgggggctggcgggcgagctggccgcgctgcccgcgcccgagcggctgcgcaggctcaccgacctggtgcgcgcgcacgccgccgccgcgctcggccaccccggacccgagtcggtgcccgccgacgtggccttccgcgacctcggcgtcgactcgctcgccgccgtcgagctgcgcaaccgggcctgcgccgcgaccggcgcgaagctcacgcccaccgcggtcttcgaccaccccaccccgcgcgcgctcgccgagcacctggccgagcacctcgccgccgccagggacagcgccgcccccgacgcggccccggacgccgaggaccccacgtccgcgctgcgcgacctggacgccctggccgccgccctgcccgccgccgcgggcgacgacgcgctgcgcgagcgggtcaccgcgcgcctgcgcgaggtcctgggccgctgggaggagctgaccggcggacccggcggacccgacgacctcgatctcgaccacatcagcgacgacgagctgttccgcctggccgacagcaggctcggcccgaccggcgcctga

SEQ ID NO: 4 - DNA, 9975 bp, Actinosynnema pretiosum
gtgtccgacgagaagaagctgcgcgactacctcaagcgcgcgctggccgagaacgagcgggtccagcagcgcctgcgcgcgctggagaccgcgcaccgcgagccgatcgcggtcgtggccaccgcctgccggttccccggcggcgtcgcctcgcccgaggacctgtgggacctggtggcgcgcggcggcgacgtcatctccgggttccccgccgaccggggctgggacctcgacgccctgttcgacggggaccccgaggcgcagggccgctcctacgtccgcgagggcgggttcctgcacgacgccaccgagttcgacgccgccttcttcggcatcagcccccgcgaggcgctggcgatggacccccagcagcgcgtgctgctggagaccgcctgggaggtcgtcgagcgcgccgggatcgacccgacctcgctgcgcggcagccggaccggcgtgttcaccggggtcgtctactacgacttcgccacccgcctgcaccccgtgcccgagggcatggagggccacctgggcaccggcatcgcccccagcgtcatctccggccgggtcgcctacgtcctcggcctgcacggaccggccgtcaccctggacaccggctgctcctcctcgctggtcggcctgcacctggccgcgcaggcgctgcgctcgggcgagtgctcgctcgcgctcgcgggcggcgtcacggtcatgtcgctgcccggcgcgttccccgagttctcccgccagcgcgggctcgccccggacgggcgctgcaagtcgttcgccgacgccgccgacggcaccgcctggtccgagggcgtcggcctggtgctgctggagcggctgtccgacgccgagcgcgccgggcacccggtgctggccgtgctgcgcggcagcgccgtcaactccgacggcgcctccaacgggctgaccgcgcccaacggcgtctcccagcagcgggtgatccgctccgcgctggaggtcgcgggcctgcggccgtccgacgtggacctcgtggaggcgcacggcaccggcacccggctgggtgacccgatcgaggcgcaggcgatcctggccacctacgggcaggaccgggccgaaccggtgctgctgggctcggtcaagtccaacctcgggcacacccagggcgcggcgggcgcggccgggctgatcaaggtcgtggaggcgctgcggcgcggcgaggtcccgcgcaccctgcacgtcgaccggcccagcgcccaggtcgactggagcgccgggaacgccgcgctggccaccgagcacgtcccgtggcccgaccgggggcggccccggcgcgccgccgtgtccgcgttcggcgtcagcggcaccaacgcccacgtcgtcctggagggcgtccccgcccccgacctgccgcccagggacgaccgcgcgctgccgctgccgctgtccgcgcgcgccgaacccgcgctgcgcgcgctcgccggtcgggtgggcgcgctcctggacgggccggtcgcgctcgccgacgtcgccggttccctcgcggacggccgcgcccggctcacccaccgcgccgtggtcgtcgccgcggaccgcgccggggcgcggaccgcgctcgacgcgctcgcggcgggcgagccccacccggacctggtcaccggggtggccgacgtggacggcaggaccgcgttcgtcttccccggccagggcaccgcctgggtcggcatgggcgcgcggctgctgaccgagtccgcggtgttcgccgactcggtcgaccgctgcgccgccgcgctcgccccgcacaccgacgtcgacctgcgcgccgccctcaccggcggccccctcgaccgggtcgagaccgtgcaggtcgccgcgttcgccatcgccgtgtccctcgccgggctgtggcgcgcgcacggagtcgaaccggacgccgtcctcgggcactcccagggcgagatcgccgcggcctgcgtcgcgggcgcgctcaccctggaggacgccgcgctggtcgtggcgctgcgcagccgcgcggtggccgagcacctgtccgggcggggcgcgatgctgtccgtcgccctgccgccgcacgaggtcgagtcgttcgggctcgcggtcgcggcggtcaacgggccggagtccgtcgtgctgtccggccccgtcgcggacgtggaggccgtgctcgccgcgctgaccgcgaccggcgtgcgggcccggcgcgtcccggtcgactacgcctcgcactcgccgggggtcgacgtgctgcgcgaggtcctggccaccgcgctcgcccccgtccggccccggcgcgcgcacgtgccgttcctgtccagcaccaccggcgcctggctggacggccccgagctggacgccgggtactggttcgcgaacctgcgcggcgcggtcggcttccacgccgccaccacgaccctgctggacgcgggccaccgggcgttcgtcgaggtcagcgcgcaccccgtgctcaccggcgcggtgcaggacagcgccgacgggcgcgcgctcgtcaccggcacgctgcgccgcgaccacggcgggctgcaccggttccggctctcggccgccgagctgcacgcgcgcggcgtccccgtcgactggaacgccgggtccggtgaccggtggcggcgcgtggacctgcccaccaccccgttccaccggcagcgctgctggccggacgcccgccgcgcggccaccgaccagccgggtctgcgcgcggcgaaccacccgctgctcgacaccgtcgtcgtgctcgcgggcgacgacggcgtggtgtgcaccggtagcgcctccacgcgcgcccacccgtggctggccgaccacgccgtgtccgggaccgcgctgctgccgggcgcctgctgggtggagctcgcggtccgcgcgggcgacgaggtcgggctcccggtgctggacgagctggtcgtggagcagccgctgccgctcgggccgcggaccacgccgctgcaggtgcgggtcggcccgccggacgccaccggccgccgaccggtcgagctgcactcgggggagggcggggactggaccaggcacgcgcgcgggcacctcgccgccgcggcgcccgtgaccgccgaggatcccgccgagtggccgccgcccggcgcgcgcgaggtcgacctgaccgggttctacgagcgccaggccaccaccggctacgggtacgggcccgccttccgcggcctgcgccgctgctggacccgcgacggcgaggtgttcgccgaggtcgtgctgccgggcgagcccggcgcgttcggcgtgcacccggcgctgctggacgcggcgctgcacgcggcggccgtcaccgcgcccgagggcgtcccggtgctgctgccgttcacctggaccggggtggcgctgcacgccaccggggcgcgggcgctgcgcgtgcggctgaccaccccgagggcgggcgagctggcgctgaccgcgtccgacggcgacggcaggcccgtggtcaccgtggactccctggtgctgcgcccggcgggcgacctgaccgccacgcgggcgccgcggtcgctgttcgaggtggtgtggaccgaaacgcccgccctgaccccggccgcgaccgcgcccaggccggtgctgcgccacgacgtccccgcggcctccgccgaggacgcggtgtgctccaccctcgccgtggtgcaggggttcctcgccgatccccggcacgccgactcggtgctgctcgtggtcaccaggggcggcgagggcccggacggcgcgaccgccgcgacgtccgccgtgcacggcctcgtccgctccgcccaggccgaggagcccggccggatcgtcctggtggacaccgacggctccgcccccgacgacctgatcaccgcgctcgccgccggaccggagccccagctcgccgtgcgcgcgggccgggtcaccgcgccgaggttgcgccgcgccgcccccgagggcgccgcacccgtcctggaccccgagggcaccgtcctggtcaccggcggcaccggcgtgctcggcgcggccgtcgcccgccacctcgtcaccgcccgcggcgcgcgccacctgctgctgctcagccgccggggccccgacgcgcccggcgcggccgacctggtcgcggagctgaccggcgcgggcgcccgcgtggtggtccgcgccgtcgacgtcgccgaccgcgccgccctcgccgccgcgctcgacgcggtccccgccgcgcacccgctcaccggcgtcgtgcacctggccgccgcgctcgacgacggcacgatcaccgcgctcacccccgaccgcgtgcgccgggtcctcgcccccaaggccaccgccgccgcccacctgcacgagctgacctccgacctggcgctgttcgccctgttctcctcggcctccggcgtgctcggcgcgccgggccagggcaactacgcggcggccaacgcctacctggacgggctggcccgcgagcgccacgcggccgggctgcccgccacctcgatcgcctggggcctgtgggccgaggcgaccggcctcaccggctccctgtccgggaccgaccgggagcgcatggccgcgtccggggtccgccccctgaccgaggccgagggcctcgccctgttcgacgccgccctcgccggaccggcgttcgtcgcggcgggcctggcggggacggcgaccgggcaggtcccgccggtgctgcgcgacgtcgtgcgcgccgcccgccccgcagccgccgccgtccgacccgcgcgcacccccgccgacctgctcgccctggtccgcgccgagtccgcctcggtgctcggccacccggcgggggagccggtcgacgcggaccgggcgttcaaggaggccgggttcgactcgctcaccgccgtggagctgcgcaaccggctcaccgccgccaccggcgtgcggctgcccgccacgctcgtgttcgaccaccccaccccggccgcgctcgccgcccacctggtggagcggatgggcgccccgcccgcgccccggccggagcgcgccgccagggccgcgtccggcgagccgatcgcgatcgtctcgatcgggtgcaggctgcccggcggggtcacctcgccggacgggctgtggcggctgctgctcgacggcgtggacgccaccaccgggccgcccgacgaccggggctgggacccggcgaccccgtaccggggcgggttcctggacggggcgaccgcgttcgacgccgggttgttcggcatcgggccgcgcgaggcgctggcgatggacccgcagcagcggctgctcctggagacctcctgggagctgttcgagcgcgccgggatcgacccgacctcgctgcgcggcaccgacaccggggtgttcgcgggcgtgatgaaccaggactacctcagccgcgcggcgggggcgccgccggacgccgaggggttcctgagcaccggcaactccggcagcgtcgtctccggccgcctcgcctacacctacgggctggaggggcccgcgctcaccgtcgacaccgcgtgctcgtcctcgctggtggccgtgcacctggcggtgcggtcgctgcgctcgggcgagtgctcgctggcgctggcgggcggggtgacggtcatggccaccaccggcctgttcgccgacttcgaccgccagggcgggctcgcggcggacgggcgctgcaaggcgttctccgccgacgccgacggcaccgcgttcggcgagggcgccgggctcgtgctgctggagcggctgtccgacgcccggcgcaacgggcacgccgtgctggccgtgctgcgcggcacggcggtcaactccgacggcgcgtccaacggcctgaccgcgccgaacggcccggcccagcagcgggtgatccgcgcggccctggccgacgccgggctgtccccggccgaggtggacgtggtggaggcgcacggcaccggtacccggctgggtgacccgatcgaggcgcaggcggtcctggcgacctacgggcgggaccggggggagccggtgctgctgggctcggtgaagtccaacctcgggcacacccagggcgcggccggggtcaccgggctgatcaagctggtgctggcgctgcggcacggggtcgtgcccgcgaccctgcacgccgggcgcgcgtcggccgaggtcaactgggaggcgggcgcggcggagctggtgaccgggacgcggccgtggccgcggaccgggcggccccggcgcgcggcggtgtcgtcgttcgggatcagcgggaccaacgcgcacgccgtgctggagcaggcgccgcccggccccgaggccgcaccggtgggcgggccgcgcgcgggggcggcctccgccggttccggcgcggtgggagcacgcggggcggagccggtgggtgtcgcgcgggccgcgggcgcgcgcgagccggaggcggggggcgcggtcgatcccggcggggtgtgtccggcgcctgcggtgggcgccgcgacgcgcgcacccgacccggaggcggctgcgcccggcgtgccgggcccggcggcgcggccggtgggcgccgggccggacgggtccgcgcggaccgtcgacgtggtgggcgcgtccgggccgctggtgcccctggtggtgtccgcgcgcgacgagcgcgcgctggccgcccaggtcgagcggatcaccgcgctgccgcacgacccggtggacgtgggctggtccctggtcaccacgcgcgccaccctgcgccaccgcgccgtgctgctggacggcgaggtcgtcgcctccggcgtcccggcgccgggcggcacggcgtggctgttcaccgggcagggcggctaccggccgggcaccgggcgggcgctgcgggcgcggttccccgcgttccgcgacgccttcgacgccacctgcgccgagctcgaccgcgcgctcgacggccgcgccgagcacccggtgcgcgccgtcgcgctgggggagcgccccggcctggacggcaccctgcacgcgcagaccgccgcgttcgccctgcagaccgcgctggccgcccagctgggcgcgttcggcctcgcgccggacgcggtggcgggccactcggtcggcgagatcgccgccgcgcacgtcgcgggcgcgttcccgctcgacgcggcgtgcgcgctggtcgccgagcgcggcgtgctcatgcgcgacctgcgaccgggcgcgatggtcgccgtcgaggtcaccgaggaggaggcgctggacgcgatcggcgacctgcccgtcgcggtcgccgtcgtcaacggcccggaggcggtcgtgctctccggcgacgccgaggcggtcgtggccgtcgcggcccgcctcggcgggcggcgcaagcggctcccggtggaccgggcgttccactcgccgcacgtcgacccggtgctcgacgccctgcgcggcctggccaccgggaccccgaccacacccctgatctccaccaccaccggccgcacgctgacccacgtcggccacgaccactgggccgaccacgcgcgcggcgcggtccgcttcgccgacgccctgcgcgccctggcgggcagcggcgtcacgacggccgtggagctgggccccgacgccgtgctcaccccgcacgccgccgaggtcctggcccgcgcgatccccgccctgcgcgccgaccacgacgaggtcgtcgccctgctcaccgcgctggccgtgctgcacgcgcgcggcgcggccgtcgactggcggcccgcgttcgagggcgcgggcgcgcgccgcgtcgacctgcccacctaccccttccagcgcacccgctactggctcgacgccgcaccccgcgccaccgcgaccggtcacccgctggtggacgcgctggacgtgctgcccggcgacgaggtcctgctgtccgcgcgctggcacgcgggccgggccggtctgggcgcgaccccgctggcggcgctgctggaaccggtgatcagggcgggcgacgaggtcggctgcccggtggtggccgagcttgacctggccaccccggtcccgctcgacgcgggcccccggcacgtccgggtccgcgtgaccgccccggacgcagacggccgccgtgcggtgaccgtccactcccgaccggacgacgagcccgcctggaccgggcacgcgacgggcgtgctcgccccggcgggccacccggaaccgctaccgcacgactccgaggcgctcgcggtggggaccaatggcgcgtggccgcccgctggggctgatgtcccggcggcgggagccaatggcgcgtccttgccccacggggccggcaccccggcggcagcgaccaacggtgcgcggtccgccctgcccgccgccggaccggcgccgggcgctgagctcgtgctgcccgacggcgccgcgcccgacggcttcggcctgcaccccgcgctctgggaggcgctgctgccgcaccccgcccgctgcgccgacgtcgtcctgcacgccaccggggcgaccgcgctgcgggtgcgcgggacctcgccgctcctggcggtcgacggcgcgggcgacccggtcgtgtcggtggggggtgtcacccgagtggcggcgtccccgcgcgccgtcacgcgggggtcgctgttcgaggtcgtcgaggtccccctgcccgcccccgcgccccccggcccgtccaccgccctgtccgctgacctgtccaccgccctgtccgcggacctgcgcggggtcacgcccgccgaggcgctcgacgtgctccagcgcttcgccgcgagcgacgcgccccgcctggtcgtgcgcaccgaccgccccgacgacccgtggcagtgcgccgtgcgcggcctggtgcggtccgcgcaggccgaggaacccggccggatcctgctcgtcgtccaggacggcgacggccccgaaccgccgcccaccacgcgggaacccgaggtggtcgtccgcgacggggcgctccgcgtgccgcgcctgcgccgcaccgcgccccgccacgacggcgtgcgcgccctccccgacaccgtcctgatcaccggcggcacgggcgcgctcggctccaccctggcccgccacctggccgccgggggagtgcggaccctcgtgctcgccagcaggcgcggccccgccgcgcccggcgcgcccgaactggccgccgacctcgcgggactcggcgcgcgcgcccacctggtcgccgccgacctcacggaccgggccgcgctcgccgcgctgctcgccgagcaccgacccggcggcgtcgtgcacaccgcgggcgtcctggacgacggcgtcctgtccgccctcacccccgcccggctggacgccgtgctggcgcccaaggcgctggtggccgcccacctggacgagctgacccgcgacggcaccacgacgctgttcgtgctgttctcctcggcggcgggcgtgctcggcacccccggccaggccaactacgcggcggcgaacgccttcctggacggcctggccgcacgacggcgggcggcgggtctgcacgcgctctccctggcctgggggccgtggcgctccggcatggccgccggggtcgacgcctcccgcgccgggctgcacccgctggacgacgccgaggccaccgccctgttcgacgccgccctcgccgacggccgcccgctgctggcgccgctgagcctggccgcgacgccgcaggacgcggacgtgccgccgctgctgcgccagctcgtgcccgtgcggcgcaggagcgcccgcgccgaggtcgccccggacccggtcgatcggctgcgcggggccgccgaccacgagctgccggagctgctgctgacgctcgtgcgcgccgccaccgccaccgtcctcggccaccgggggcctgagtccgtcgacccggacgagcccttctgggacaccgggttcacctcgctcaccgccgtcgagctgcgcaacctgctggccgagcgcaccggcgtgcgcgtcaccgccgccgcggtctacgaggagccgaccccgagggcgctgtccgcccacctgcgcgccgcgctgcgcgcgccccacaccagcgggacggtctga

SEQ ID NO: 5 - DNA, 726 bp, Actinosynnema pretiosum
gtgccgctgcccgccgggaccgggcgggacgcggccggggtggtggaccgggtcggcgcgggggtcgacggggtcgcggtcggcgaccgggtgttcggcgaggggatcgagacctgcgcggagttcgcggtgctgacggcgtgggcccacctccccgagggcctctcgttcgaggaggcggccgggtacccgtcggtggtggagacggcgctgcgggtgctgcgtgaggtcggggtgcgggaggggcagtcgctgctggtcagcggcgcgtccgggggtgtcgggtcggcggtgctccagatcgcgcgcgaccggggcgtgcgggtggtcgggaccgcgagcgcggcgaaccaggactacctgcgcgacctgggcgcgaccgcgacgacctacgggccggggtgggtcgagcgggtgcgggcgctggtcccggtggacgcggcgctcgacctggccggggcgggggtgctccccgacctggtcgcgctgaccggcgacccgcggaaggtgatcaccatcgccgaccgggccgccgccgagcacggcgtccggttctccggcgcggcgcacgacgtgcccgccgcgctggccgaggcggtcgacctgatcacgaggggcgccctgcgcatcccggtcgaacgcgcctacccgctcgccgagacggcgcgggcccacgccgacagccaggccgggcacgcgcgggggcgacgggtgatcgtgatctga

SEQ ID NO: 6 - DNA, 645 bp, Actinosynnema pretiosum
gtgaccgcgccaccgggccgtcgcgagcgcaagaaggccgcgacccgtcagaagatcgccgacaccgccctgcgcctgttcctggagcgcggctacgacgcggtcggcgtccgcgacgtcgcggccgaggccgacgtcgccgtcaccacggtcttcgcgcacttcgacagcaaggaggccctggtcttcgagcgcgacgccgacttcgagcaccgcctcgtccgggccgtcgccgagcgcgggcgggacgacctcgtccccgcactgcgccacgaggttcacgcgatcgtgcggcactgcaccaccgacaggtcggccgcgctgggggagttgatccagtcctcaccggacctgcgcggctacaacgaggccatgtgcgcccgccacgccgcctccctcgcgaccgccatcgccgccgaactcggcctgccgccgcgcaccacggcctgccgggcgatcgcccgcttcgcggtcgacgcccacgtcctcgcgcgcgaggcccaggaccccgaggcggccgtggacgaggtcttccggatgatccaggccgcctgggacgccgccgacctcagcgccgacccccacgccgacccccacgccgatccccacgccgacctcagcgcgggcgcgccgcaggaccgctga

SEQ ID NO: 7 - DNA, 267 bp, Actinosynnema pretiosum
atgcccttcgtcgctcctctcctgctcggtctcctgaccgtccccgtggccaggagggtcgccaagccgctcgcccgcggcgtcgtccggacctccgcgggcatcgtcgccggtgcccgccgcgccgcgcacgccgtcggcgaggagctccaggacatcaccgccgaggtgaccgccgagctggaggccgcccggctcgccgccgacgtggtggagcccccgaggggcgcgcccgccgggaccgggccggacgccagagcgcgctga

SEQ ID NO: 8 - DNA, 2247 bp, Actinosynnema pretiosum
gtgacccagctcctcgacgccgccccgaccaccaggctcaccgggttccgggtgcgctcggtcgtccccggccgccagcgctgggacgtcccgcgcgtgcgcaggaacccgcgcgccgcgcgcgccctcgaagccgcgctgctccgccagcccggcatcaccggggcgcaggccagccccgcgaccggccgggtgctggtgctgcacgaccgggcgctcggcgtcgccgaggtcggcgccgccctgcgggacgcggtcgccgccgcggccagcgccccgctggtcgtcgagggcagcaccacgacgagcccaccggccaccgccgcgagcacgtccggcggcacgtccgaggtcgcgctccggcccgcgagcgccgtcctgcgccgcccgctcgccctggtcggcgtggtcaccggcgtctcgtccgccgtcctgcgccacgcctggcgcgccctcctgcggcccctgcgccgcaccgccgcgggcacccggctctccgcccacccgctggtgcgcgccctcggccgccgccggtcggcgctcgccgccgccgccgcgctgacggtggcctgccagctcgccgagctcggcctcgggctgttcctcggctggatcctgctggtgctgatcaagggcgagtacgccccgctcaccgcgctgggcgtcacgagcgcccccgcccagctgctcgcgctcgccgccggggccgccgcgacctgcggggtcgtcgtcgtgctctccgccgcctccggcgcccgctggcgcgcgctcggccagggcgtccggcgcgaccagcgcgcccgcctgcaccgccacacccagcgcctggagatgcggcacatccagcgcgagcggcccagccgcgtcgcgggcgtgctcgccgacgacgtcgaccagctcggcgcgttcttctccggcccggcgggcgaggcggtccagctcgccaccagcgcgctgatcctggtcccgtcgttcctggtgctcgtgcccggcatcgcctggatcgcgttcctgcccctgccgttcgtcgcggccctgtccctgcgccagcgcgaccgctccgccgccgactacgccagcgcgggcgagcaggccgccctgctgcgcagcaggctctccgccgcactggaggccgccgccacggtcaagagcgcctgcgccgaggaccacgaggccgagctggtcgacctgctggagctggcgggcgccgacgcggccacgcgcacccagcagcacgccgtccggttcggcgagggcatccgcgcctggaccacctgctccatggcgggcaccctgctcgtgggcgggctcaaggtgctcgacggcacgctcccgttcgagcggttcagcccgctgatcgggctgccgcagcagatggtcatgcggctgacccggctgggcccgatcgtcgaccagtaccagcgcaccctcgccgcgcacgaccgggtcgagcacctgatgtccctgcccaccgagcccaccggtggccgcgcgctggcccgcgagcgggtcgcgggcgccatcgcgctcgaccgcgtgtccttctcctacgagggccgcccgcgcgtgctggaccggttgtcgctgcgcgcggcggcgggccgggtcaccggcatcgtcggcgcgaccggctcgggcaagaccaccgtcgccaagctgctgatgcgcttccaggacgcggacaccggccgggtgctgctcgacgggcgggacgtgcgcggcctgcgcctgcccgacctgcgcgcggccgtcgggttcgtcgcccaggacccgttcctgttcgacggcacgatcgcggacaacatccgctacggcacgttctccgccaccgacgagcaggtccgcgaggccgcccgcacggccgaggcccacgcgttcgtcgagtcgctgcccctgggctacgacacggtggtcggcgagcacggcgcggcgctgtccggcgggcagcggcagcggatcgcgctcgccaggaccgtgctgaagaacccgcccgtggtggtcctggacgaggccacgtcggcggtggacaacgagaccgaggcggcgatccagcgcgcgctggcgacgttcgcgcggggccgcaccctgatcgtgatcgcgcaccggctctccaccgtccgcaacgcggaccacatctacgtgctggagcggggcggcgtcgtcgagcagggcgcgcacgagggcctggtggcctccggcgggcgctacgcctcgctgtgggagctccaggcgggggcgaggggctga

SEQ ID NO: 9 - DNA, 1251 bp, Actinosynnema pretiosum
gtggcggccccggcgggcgactcgctcgtcgcgctgctgttcctcgacctcgcggtcgtcctggcgctcggcgccctgctcggcgcgctggccaggcggctggggcagccctcggtggtgggggaggtcgcggccgggatcgcgctcggccccagcctgctcggcctgctgcccggcgacccggtggcgctgctgttcccgcccgaggcgcgcccgccgctggcgttcgccgcccagctcggcctggtgctgttcatgttcctggtcggctgcgagctggacgtcgcggagctgcgcggcgcggggcgggtggtcggcgcggtgtcggcgggctcggtcgtgctgccgttctgcctcgggctgggcgtcgcggcgctgctgtggccgcagggcggtgacgcgctcgcgctgttcaccgcagccgccctgtcgatcaccgctttccccgtgctggcgcggatcctggccgagcggcggatgcagcgcacccggtccggggtggtcgcgctggcgagcgccgcggtgaacgacgtggtggcgtggtgcgcgctcgccgtcgtcgccgggatcgtgaccgcgcgcggcccctggtcggcggtggccacgctggcctggacggcggcgctggtcctggtggcggtgctcctcgtccggccgctggtggggtgggttgcccggacggtgggcggctcgccgcgcgcggacgcggtgctgttcgcggtcgtcgtccaggggctgctgctgttcgcgctcgccaccacggccatcggcctgcacgcggtgttcggggcgttcctgttcggggcggttgtgcccaaggacgcgctccgggaggccgcgccgaccctggtggaccgggtcggcgggctcagctccctgctgctgccggtgttcttcgtcgtggccgggctgtccgtggacgtcggcggcctcggctggtccggcgcggcggaggcgctgctggtgctcgtcgccgcgtgcgtcggcaagctggtcggcgcggcgggcggcggtctgctcgcggggctgcccgcgcgcgacgcggccgaggtcggcgtgctgatgaacgccagggggctcaccgagctggtggtgctgggcgtggggctggagctgggggtgctggacgggcgcctgttcaccgtcctggtcgtgatggcgctggtgacgacggcggcgaccggcccgctgctgacgctgatcgcccggcggtccgggcgggaggtcgccgcgctcgaaggactccggtag

SEQ ID NO: 10 - DNA, 294 bp, Actinosynnema pretiosum
ttggcgggcttgccggtcccgaccaccgccagaccgaactggttggtcagcgggaaccgcgccgggctcacgaacgcgacgcaccagacgtcctcgccggccaggcgcctgccctcgaaggcgaccgactccagcgcggtcaggcagttctcggcgcccgcgtccgcgatggcgaccgtcgtcgacccgctctcgctcgacgtcgctcggaacgcgggtcccgccaccggggcgcggaccacgccgaccaccgggatcgtgctccgcgccctgcggctgtcgtgcggccggtag

SEQ ID NO: 11 - DNA, 1047 bp, Actinosynnema pretiosum
gtgacgttgaccgacgacgagcacaccgcgcaccggcacgacgagggcgccgcggccttccaggcgctcgcgaccgccgccgcaggctggggcgcgctgcgcgccgccgtggagctgcgcctgcccgacctgctcccggacgcccccaccgccgtccccgacctggcggcgctggcgggcgtcgcgccgacgcggctcgcgcagctggtcgcggtgctcgccgacgtgggcgtgctcgcgctggacgagcgcgcccacgtccgccacacgccgctctcccgcgccctgcgctcgaccgacggcctggcgctcacccgcctgctcacggcgggctgggcggacgaggcgtggcgcgggctcgcggagggcgtgcgcaccgacacctccccgctggagctggtgcacggccgcccgctgttcgacctcctcgccgccgacccggccgccgccgcggtgttccacgacgcggtggccaccggccccggcgacccgttcgacgaggcgatccgctccgcgctcgacctgaccggggccgcgcgggtggtcgacgtcggcggcgggcggggcaggctgctcgccgcgctgctgcgcgagcaccccgtgctctcgggcgtgctgttcgacctgcccgaggcggtccgggacgcggttcccgagctgcgcggcgacgaccgggtccagatcagggcgggcgacctgttcgcggaccgggtcggcgcggcggacgtctacgtgctgcgcacggtcctgcacgtgctggacgacgcgcaggccgtgcgggcgctggccaacgtccgcgccgacgcgcccgcgcacgccagggtcgtggtcgtggaagcgctttccgacgacgaggccaaccggacctacgcggcgcacagcagcctgcggctgttcgtgctgtgcggcgggcgcgagcgctcgacggcggagttcgccgagctgttcgaccgggccgggctggagctgcgctcggtcaccccgaccgggacgccgtaccacctgctggagggcgccccacgcctgagcccgcgcacggacgggtga

SEQ ID NO: 12 - DNA, 3354 bp, Actinosynnema pretiosum
atgcggctggtggaccgcgacgggcagtgcgaggcgctggctgccctgctggagggcgcggcgggcgggtgcgggggcgcggtgctgctcaggggcgcgccggggcacgggaagaccgcgctgctggggcgcctggtcgtgatggccgagggcgcggggttccggtgcctggtggtcgaggcggacgcgctgcccgaggtcgcggaggtcgcggcgggcggcggcgcgctgctggtggccgtggacgacgccgggggcgccgacgtgggcgcgctgttcgacctggtggcgtgggcgagacgcacgaccgccgtcctcgcgctcaccgacctgccccgcctgcccgacgacgacccgctgggcgccgtggcgcgcacgccgggcgccctcgtgctcgacctcccgccactgcgaccggagaccaccgccgcgctgctcggcgggtccccgtggccggtgaccggggggaaccccgcgctggtgcgcgggctggcccacgacctcgtggcgggggaggcgggaccgggggccgcctaccgcgccgccctcgccggggtgctcgaccgctgcggcgccgaggtggccgccgtcgcgcacgccaccgccgtgctcaccgggcccgccgttgggcttctcaccgcacccgccgccgcgcttcccgccgggcttcgcgccgcacccaccgccgcgcttctcaccgcacccgccgccgtgcttcccggcggggtgcttcctggtgccgcgcttcctgacggggcgcaccctgctgcgggccctggtgaggcgtgcgaaggcaccgcgcctgctggcgaggctctttccgctgctgtgcgccctggcgaggcgtgcccaggcaccgcgcttcccgacggggtgcttccgggggtcgcgtctcccggcggggcgttccccgccgccgacgttctcggcgggcccttccccgccgccgtgcgttccgccgccgcgcttccagactgggccctccccgccgccgtgcgtcccggcgaggcgtgccctggcaccgcgcttcccaccgacgcgccccctggggccgcgccccctggcggggcgcttcccgacggcggggcgcgaccgctcacgtctggcgaggtgggcgtccggcaggggggagtggggccggtcgcgcccggtcgcgccggtgcctcgccggggggcgctgagccgctcgtgcccggcccggtgtctggggtgagggctgatcccgccgggggggacggggcttccgcgttcggtcgggtggctgcggatcacgtcgttcccgctggggtccgcccccgcgcgtcctcggaccccgccgcgcccgtcgacctggtcgccgacctgctcggtcgggcccccgcctccgtcgtcgacgcccgcgcccgcctcgcgggagccgggctcgaccgggccgatcccgccgctgtgctggacgtgctgcccgccgcccggcgcgccgagctgcacctgcgcgccgcgcggacgttgcacgagcgcggtgagtcgcccgccgtcgtcgccgaccacctcgtcaccgccgggctgcccgcgcccgcctgggccgtcgacgtgctcgtcgccgccgccgaccacgcgctcgccgccgacgacgtgcccaccgccgtcgcacggctcgccgccgcccaccgccgggacgacgcggaccaaccgcgggccgtccccccgcgcgcagtcccggacccgcccgtcgccgcccaccggtgcgggtgcgcggaccggacggccgcgcccgccgagcgccgcacgtcgcccgaggccgccgccgtcgccgggcccgcgctcaccgcccggctcgcccgcgcgcgctggcagctcaacccggctgccgcgctgtgccacctcgacccgctcgccgacgcgctcgccgccgaccccgacgcgctcgccccgcgggacgccgccgcgctggtgcgccagctggtctggcacgggcgcaccaccgccgccgggcgcgtgctcgccgcgctgcgcgcccgcgagcacgccgccgagctgcccgacctggagacctggctctccctcgcctgcccgccgctcaccgcctcccgccgccccacaaccctccacgcctccgccccgcacacctccgccccgcgcacctcggcccaccccacccccctcgccccacccccgagccgcgtcgacccgtggttgcgcgaggccgccagcctggtcggcgggttcgcgctcggggaccagcgccgggccgccgccgaggccgcgcactacctgcgccacgcccacctcagccacacctccccgtggtccgaggaacccgcgctgctcgccctgctagccctggtgcgcgccgaccgcgccgccgacgccgaccgggcctgcgcccggctgctcgacgaggcccgcgcccggcgcgcgcccacctgggtcgcctccttcgccgccgccgcctcggtgatcgcgctgcggctcggcgacctcgcgggcgccgccgggcacgcccgcgccgcgctcgacgccgtcccctcgcacggctggggcgcgtccgtgggcctgccgctgggctcgctgatcctcgccgacaccctgcgcgggcggcgccgggaggccgggcggcacgtcaccgcgcccgtgccggacgccctcttcgacggccccgccgccccgcactacctgcacgcgcgcggccaccaccacctcgcgtccgggcgggaccacgcggcgctcgcggacttcctggcgtgcggcgacctcctgcgctcgtggggcctcgacctgcccggcctcgcgccgtggcgcaccggggccgccgaggcgtggacccgcctgggcaaccacgaccgcgcccggcggctggtgttcgaccagctcgccacgaccggcgccgacgcgggccgccccagggcgctggcgctgcgcgcgctcgccccgctcagcccgccgacccgccgcccgcacctgctcgccgaggccgccgacgccctggagcgctgcggtgaccgctacgagctggccgccaccctcgcctgcctcggcgccgcgcaccacgccctcggcgaccaccgccgcgcccgcgccgtgctgcgcagggcctggcacctggccagggcgtgtggcgcgcgaccgctgtgcgcgcgcctggaaccggacgcggccgaccccgtcgagcacgacaccagcggcctcaccggctccgagcgcaaggtcgccgccctggccgtcaccggcctcaccaaccggcagatcgccgagaagctgttcgtcaccccgagcaccgtcgagcagcacctgaccagggtgttccgcaagctgcgcgtcacccaccgcgccgacctgcccaccaccctgcacgccgacctgaccacccccgcctga

SEQ ID NO: 13 - DNA, 780 bp, Actinosynnema pretiosum
gtgtacgacaccaacacctacctggcccggctcggcgtcgaggtcacccgccccgaccgggccgcgctgaccgcgctgcaccgcgcccacctgcgggcgctgcactacgacaacaccgccgccgccactcaggacggccccgttcccgacaacctcgccgacctggacgtggacgcgaccttcgacgggctcgtcacggccgggcggggcggcatctgcttcgagctgaacctgctgttccaccggctgctcaccgacctcggcttcaccacgaccgtgctgtcggcgggcgtggccgacgaggagggcggcttcagccccgacctggcgcaccggttcaccgccgtgcacctggacggcgaggtgctgctggccgacgtgggcttcgccggaccgtcctacctggacccgatcaggctggccccggacgagcaggtccagcacgggtgcgcgttccgggtggtggagcaggacggcaggcacctcgtgctgcgcaggagccgcaccaccgactggcgcccgctctacgagttcgccaccacccccaggacgctgtccgactgggacggcttcaccccgagactgcgccgctacctggaccgcgcggtgatcgcgggcaccaccctgctgtgccgcgcggtcgacgacggccaccgggccctggtgggcaagcggcacctcgtggtccgtgacggccacgagacggtgaccaccctgctggacccggccgagcacgcgcgcgtgagcgccgagatccgcaccgggatctgcgccgactga

SEQ ID NO: 14 - DNA, 885 bp, Actinosynnema pretiosum
atgagcctcccagcggactcaccaccgccggggccggtggagatgcccgacgagagctggacgtcgctgggcaacgcgggggcgcgggcgcaggagtccgcgcgggccgaccggttgttcgacgacccgctggcgcgggcgttcctggacgcggcggggacggggaacccgctgctgcccgaccggggtggcggtgatccggggctgctcgggcagatcacggacgtcatcgtggtcaagacggtgttcttcgacgcggtgctggcgcgggcggcggcggccggggtgcggcaggtggtgctgctggccgccgggttggacgcccgcgcgttccggctgccctggcccgagggggtggtggtgttcgaggtggacctgccggacgtgctggggttcaaggagcgcgtggtgcgggaggtcggggcggagccctcgtgcgaccggcgggtcgtcgcggcggacctgcgctcggactgggtggccgcgctggtcgccgccgggctggaccgggaggccccggtggcctggctggcggagggcgcgctggggctgctggacgaggccgggtgcgaggagctgatggcggcggtgctcggggcctccgcggcggggagccggttcgcgctcgaccacacccacgacgggtggaaggccggggaggcgctgggcgggtacctggaggggaccggggtctgcttggccgacctggtgaaggggggtccgcgggagccgggcggggcgtggttggcgcggcacgggtggcgggtggcggagtacgacgtcgtggcggaggcggcacggcacgggcggcccgcgccgaccctgttccgggtgccggagcggcgggcgaacgcgacgatcctgttcgaggccgagctggggtag

SEQ ID NO: 15 - DNA, 1443 bp, Actinosynnema pretiosum
gtggtcgaccgggcgggcggtcccgcgaccacctcgcgggcgctgggcgtgcagccgcgcggcgtggaggtgctgctgcggctgggcgcgctgggcgacctgccggggctggtgatcccgctgcgcgaggtgcggatgcacagcgagggcaggctggtgtcggtgatgccgatggcgcgcatggcgcaggcgtcggggcgcgcgccgctgctggtgtcccaggtggaggtggaggcgtcgctgcgggagcggctggccgcgctgggcggcgcgccggagtggggcgtggagctgctggactgcgcgcaggacgcctccggggtgaccgcgctgctgcgcgacggcggcggtgagcggcgggtgcgggcggactgggtggtcgggtgcgacggcgcgcgcagcctggtgcgggagctggcgggcatcgggttccccggcgagcaggtggccgagcacttcctgatcgccgacgtgcgcgcggactgggcggtggaccggggcgtgatgggcagctggacgcgcgggcgcgacatggtcagcgtgtgcccgcttcccggcgaggaccggtggcggctcatgggccccgcgcccgccgggacggccgggcggctgcgcgacgacgaggtgcgggagctggtgctgcgccagctcgcgcgctgcacgccgcaccccgccgaggcgatcacgcgggtggagtggctgtcgtcgttccggatccaccgcaggctcgccgaggcgcaccgccgggacaggctgctgctggccggggacgccgccgcgatccaccacccgttcggcgggcagggcatgaacaccgggctgggcgacgcggagaacctggcgtggaagctggcgctggtggcgagcgggcgggccgggcaggagctgctggacacctacgcggccgagcgcaggcccgtcgcggcgggggtgctctcggggaccggggtgaccaccggcctggtcgcggcggacaacccgctggtggcgctggtgcgggaccgggtggtgatgccgctgctgaacacccggctcgcgcaggggttcctgtggcgcagggcctcccagctgggggtcgggtaccggcgcgggccgctggccgctccgggcgcccggcggggatcggggcccgtgcccggcgaccgggtgcccgacctggcgtgccacgacgggggcgggcggacgacgctgcacgcggcgctgcgcggccggtgggcggtgctggcgccggacggggtgggcgggtcgaccgtggaagcggtgcgggggctgctcggcgtggacgccgtggtggggctggcgccggtggcgggcgggttggcggaggtgctgctggtgcggccggacgggcacctggcgtggcgcgggcggccggaccccgagggggtcgcggcgtggctggggtcggcgctgagcctggtcggggagggggcgctgcgatga

SEQ ID NO: 16 - DNA, 1326 bp, Actinosynnema pretiosum
atgctcgacgcgatcgtgatgggtggtgggcccgccgggtccgtctgcgccgcggtgctcgcccggcagggccgctcggtgctggtgctggagcggcaggagttcccccgcttccacatcggcgagtcgatgctgccgtacatggtgggcctgctggagcggcacggcctgctcgacgcggtgcgggagcagggctacgtcgtcaagcgcggcggcgagttcatcgacccgaccggcacgaagttcttccgcgccggggtgttccgcgccgacttcgccaagaccggcgacggccgccaccacgagacgttccaggtcgagcgctcgcacttcgaccgggtgaacctggaccaggcgcgcgcggcgggcgcgaccgtgcgggagggcgcgcaggtcgtcgggctgctggaggagggcggccgggtggtgggcgtgcgctaccgcgagggcggggtcgagcgcgaggagcgcgcccggtacgtggtggacgccaccggccgggcgggcgtggtcgccaaccggttcgggctgcggcggatgatcgaggacctgcgcatggtggcggtgttccaccaccgcgacgggctggacgaggcgcacaaccccggccacgagggcgacatccaggtcggcagccactcggacgggtggatctgggcgatcccgctgtcggcggaccggatcagcgtcggcacggtcatgcaccgcgaccggctgcgcgggcgcaccccggccgaggcgttcgccgagcacgtcgagcgggtgcccaggatcaaccagcggctcaccggcacctccgcgacctcggacttctgggtggagaccgactacagctaccactccgaccaggtcaccggccccggctgggtgatggtcggggacgccggctgcttcggcgaccccatgttctccggcggggtgctggtgggcatggccacgggcgcggaggccgccgaggcgctcgggcgggcgctcgactcgcccgccgacgaggagcaggcgctcaccggctactcgaacttcttcaagaccggctacgacacgtacgtgcggctgatcttctccttctacgagggcgagctgctgcccgcgctggccgaggcgcactcggaggcggggaacctctccgaggcggacatggagatgtacgtggtgcggctgctcggcggcgacttctggagcgcgcgcaacccggtggcgaacgcgctgcgcgcgaacccggcgtggtcgacgttctcgccgttcgagccggtgcaccgctgcccggtgtaccccgagctggacgtggcggagctgggcgggctcgcgggcacggtcggccggtga

SEQ ID NO: 17 - DNA, 1026 bp, Actinosynnema pretiosum
gtgcccggcgacaccgacgacccaccggcccgagcgctcgccagcccgtcacccgacggcccgccacccgacggcccgtcgtccctcggtccgtcctccggcggcatgtcgcccggcggcgcggcgcgcggcggcgcgtcgcccggcgaaccgggccgcccggccggggagcgcccgctgctggtgctgggcgcgggcgtcatgggctgcgggatcaccgcgctcgcgctctccaggggcctgcccgtgctcctggtggacccggacgccgaccggctcgacgcggcccgcgccgacgtgcgcgcgcacctgcggaccgcccagctgctcggcgtcgccgccgggccgctcggcgagctgaccaccgcgaccgacacgggcggcccccgcgaggtggtcgccgtggtcgaggcggtcaccgaggacgccgagaccaaggccaaggcgctcaccggggtctgcgccaccgtcccgccgggcaccccgctggtctccaacacctcctccatccccatgggcgagctggcccccgcgctgcccaggcccggcgacctcgtgggcgcgcacttcatgaacccgccgtacctgatccccgccgtcgaggtagcgcgcggcccgctgacctcggacgcggcgttcgccgggctcaccgccctgctcgcgcgcctcggccgcgccccggtacaggtcggcgacgcgccgggcttcgtcaccagccgcctgctgcacccgatgatcaacgacgcggcgcgcgtcgtggagtcgggcaccgccgacgccgccgccgtcgacgcgctcatgcgcgactgcctcggccaccgcgagggcccgctgcgcaccgccgacctgatcggcatcgacaacctcgtcgactcgctgcgcgtgctgcgcaggcgcaccggggacgagcggtgcgagccgtgcgacctgctgctggccaaggtcgacgcggggcacctcgggcgcaagagcggccggggcttctacgactacggccaccccggccgtgaagggacggggcgatga

SEQ ID NO: 18 - DNA, 273 bp, Actinosynnema pretiosum
atgacctcgggcacggagtcgcggaccgacgacgtccagggcgcggtgctggacttcctgcgcgcccgcaccggcgccgagtgggacgccgagcgggacctgttcgccgagggcggcctgtcgtcgctgttcgcgatggagctggtcgtgcacctggagcagtgcttcgacgtcgagatcgcgggccccgacctgcggctggacaacttccgcacggtgcgcgccatgaccgcgctcgtggaccggctccgcgcgggcggcgccggtggctga

SEQ ID NO: 19 - DNA, 1074 bp, Actinosynnema pretiosum
gtggtcgccgaccgggcgggggagtgggacctggcgggcgagctgccggtggccgcgctccgcgagctcggcgcggcgggcgcgctgtgcggtgaggtcgccgagcggcacggcggcctcgggctcggcagcctggacaacggcgagctgaccgcccgcaccggcgcgctgtgcggctcggtgcgcagcgtccagacctcgcagggcatggccgcctggaccgtgcgccgcctcggcgaccaggcgcagcgcgccgccgtgctgcccaggctcaccgggggctcgctggccgccgtggcgctcagcgagcccggcgcgggcagcgacctgtccgccatggccaccaccgtcgaggacgaccccggcgaaccggggacggtcctggtgcgcggccacaaggtgtggaccaccgcggcccgctacgccgacctgctcctcgtggcgggcaggcgcggcgacggcgcggcggtcgcgctggtgcccgccaccgcgcccggtgtcacgatcacccccgtcgccaacccgatgggctgccgggccgcgggccacgccgacgtcgagctggacgacgtgcgcctgcccgccacctcggtgctgggcggcgacggcgtctccaccgcgctgctggtcaccacggtcctcgcgcacggcaggctgtccgtggcgtggggctgcgtcggcatcctgcgcgcctgcctggccgcgtccgccgagcacgcccgcacccgccgccagttcggcgtgccgctggccgcgcaccagctcgtcgcgggccacctcgccgacctgctggtcgccgagcaggtctccaccagggtgtgcgagcacgccagccgccgctgggacgagagctcggccgagctggtcaccgccaccgtgctcgccaagcacgtgagcgcggggcacgcggcgcgcgccgccgccaccgccgtccaggtgctcgcctcggcgggcgcccacgacggccaccccgtcgcgcgcgcctaccgggacgccaagctcatggagatcatcgagggcagcaacgagctgtgcaggctcgtgctggccgagcacgcggtggcgacggcgtga

SEQ ID NO: 20 - DNA, 1314 bp, Actinosynnema pretiosum
gtgctggacgagcgggtccgcgccgcgatcgtcgagctggacgcgcgcggcgtgctccagtcggtggccagccgcaacgaccccgagcccgccctcgccgcgctggaggcgctgggcgtcgccgagtacttcgtgctgcccgagatcggctggggcgtcaagtccgccgccgtcgcccgcatcgccgaccggctgggcttcgcgcaccgcgccatcgcgttcgtcgacgaccggcccgccgagcgcgccgaggtcgccttccacctgcccgaggtccgctgctacccggccgagcgcgtgctggagctgtgctcgctgcccgagttcagccccgccgtcgtcaccgaggacgcccgcaaccgccgcgccctctaccgggccgggttcgcgcgcggcgccgagcaggccgcgttcaccgggcccgacgaggagttcctgcgctcgctgggactggtgctgcggatcgaccgggccaggcccgaggacctgcaccgggtcgaggagctgaccgcgcgcaccagccagatgaacgcgaccggcgtgcactacccgcacgggcagttgcgcgccctgctggacgacccggcgcacgaggtgctggtggcgggcctgaccgaccggttcgggccgcacggcgcgatcggcatcgtgctgctggagcggcacaccgccgcgtggcacctgaagctgctggcgacctcgtgccgggtggtgccctacggcgtgggcagcgtgctgctggactggctgtgcgggcaggcgctggaggcgggcgcgcacctggtggccgacttccggcgcaccgaccgcaaccgcatgatggagatcgcctaccggttcgccgggtacgacgacgccgactgcgcctgccgcgccgagctggcggcggtgggcggcggccccgaacacctcgctgcggcaggcgccccaggcgcgggcgctccggggatcggcgcctcgggagccggtgcttcaggagccggtgctcccgcgggcggtgctcccgcgggcggtgctccaggaaccggtgctgccgagactcgcgctgccgagcctcgcgctgccgagtctcgcgtggctgagccccgcgctgccgagtctcgcgtggctgagccccgcgctgccgagtctcgcgtggctgagccccggaccgccgagccccgcgccgctgatgtccccgtcgccgaggttcccgaggcgcgtgccagcgggaacccggtgcggcggctgcacctgcgccccgcgccccgacccgccccgcgcgcggtcaccgtcgaagccccgtcgctgctggaggtgcccacccgtgggtga

SEQ ID NO: 21 - DNA, 504 bp, Actinosynnema pretiosum
atgctggtcgcgctcaccggggcccggctggtcgtggaggtcggcaccttcaccgggtgcagcacgctgtgcctggcctccgccctccccgacggcggccgggtgatcacctgcgagctgagccccaagtggcccgcgatcgcccggccgcactgggagcgcgcgggcgtcgcggaccggatcgaggtgctcgtcggcgacgccgcgggcagcctcgccgggctgaccggcgagctcgcgggccgggtcgacctggtgttcgtggacgcggacaaggccgggtacccccgctactacgagctggccgtcgagctggtgcgccccggcgggctggtcgtcgtggacaacaccctgttctccggccgggtcgccgaccccgccgcggtggaccccgacaccgagggcgtccgggagctcaaccgcaggatcgccgccgacgaccgggtccaggccgtgctgctcgcgcacgccgacggcatgaccctggcccgtcgcacgcgctga

SEQ ID NO: 22 - DNA, 2742 bp, Actinosynnema pretiosum
atgcgctttcagctcttgggtccgctcgaagtcgtcgaccggtcccgaccggtggcgctcggcggcaccaagcagcgcgccgcgctcggctacctgctgctgcacccgaacagcgtggtgcccaccagcagcctgctcaaggcgctgtggccgcaggacatgccgcccaccgggcgcaagatgctccagaacgcggtcgccgccctgcgcggcgtgctgaccgccgccggtgacgcgcccggcgcgcccgccctgctcacccacacccccggctacctgctgcgcgtcgacccggaggacgtggacctgagccgcttccgcaggctcgtccacgccgggcgcgccgacctcgcggcgggctcctggcagcgcgccgcccgcaccctgcgcgacgcgatcggcctgtggcgcgggccgctgctggccgacctggtcgagcagggcgtcgactggcccgagctggcggccgtgcgcaacacccggctggccgcgctggaggactgcgtcgaggcggagttcgcgtccggccgctacggcgagatgatctacgagctggagcggtgggtcgaggccgagccgctgcgcgagcggctgtgcggccagctcatgcgggcgctctaccactgcggcaggcaggccgacgcgctcggcgtgtaccggcgcaccagggccaggctggtggacgagctggggctcgaccccggccgcgagctgcgcgagctggagcgggcgatcctcaaccaggaggtgccgcaccggctcccggcccggcgcgaggcggcgcccgccgcgcggtccggggcgaccgagggcggtggcccggtcgcggcgcccccggtcgcggcgcccccggccgggatcgtcccgcagcggcaggcgcccccggcgcccgcgcctgcccccgtcgcgctcgcgcccctccccccggcgtccgcgccgaccgccgagctcaagcacgtcagcgtcctcatggtcctcacccggttcgagcacgacgtggccgccgccccgcccgaggaggtggacgacgcgctgagcgacgtcgccaccgcggtgcgcgaggaggtcgagcgggcgggcggcgtcgtcaccgccaaggccgcgtcgctgatcgtcgccctgttcggctccacccgcagcggcgaggacgacgccaccagggcggtgcgcgccgcgctcgccgtgcgcgaccggctcgcccacgggccgcgcggcgccgggcccgccgcgctgggcgtcaccgccgccgtcgccaccgggcacgccctggtgcggcgccacccagggcgcgcgcccgaggtcggcggcggcgtgctcgaccgctgcatgaggctgctcgcgcacgtctcgcccggcgaggtcagggtgtgcgaggagaccaggggcgccagcgagggcgccatcggctacgacggcccccacgacccggtgcgcgggtcctgggccaccggggccaggcagggcgcgggcgggcccccggccacgccgccgctggtggaccgggaccgcgagctggccctgctgcgcggcctgctggagcaggtcggccgcaggtcccggacgcacctggtgaccgtgctcggcgagcccggcatcggcaagagcaggctcgtggacgagttccagcgcgtggccgagacctcggcgaccgttgtggtccgctgcccccgcctcggcggagggggcgacctcgccccgctcgccgacgcgctcgccccgcgcatgggcatcggcccggccgacccggtcgggctggtcgccgccaagctcgccgccgccgcgcgcggcagcggtggcgcgggcgagcgcgcggaccgggtgctggccagcctgcgcgtcctgttcgggctcgacgtgcccgacgtgctcccggacggctcgttcgcggcgtggcggcacgtgctggaggacctcggcggcgcggacccgctcgtcctggtcctggaggacctgcactgggcgggcgaggcgctgctggacttcgtggaccacctcaccgccgaggccacgtcgtccccgctgctggtcgtggtcaccgcgcgccccgagctgttcgaccggcgccccgcctggggcggcggcaaggccgaggccagcacgatcaccctcgacccgctcgccgaccaggacgtggagcggctgctcaccgccctgtgcgtccggcgcggccccgccaccgacgaggcgagcgccgcctaccgcgcggtgatcgcccacatcggcgggaacccgctgttcgccgtcgagtacgccagggtgctcgccgacgcgggcccgtcgtccgggggcggcgggcagtcgccgccgctgccgccgctggtgcgccgggtcgtggcggcccgcatcgacagcctgcccgccgacgccaaggcggtgctgctcgacgcggccgtcctcggcgaggacctgtgcgaggagggggtgcgcgcgctcggcggcggcgccgtgcccgacgtggcgcgcgcgctggagcacctggagcggcgcgaggtcctgcgccgggaccgggacgccgagcgcgccaccgcctacgcgttccgcagcccgctcgtgcgcgacgtcgcctacctgaccatcccccagcacgcccgcgccgagaagcaccggcgagcggccgactggttggagcgcgtgcccggtcacgacgcgctgcggctggtccaccacgggctcaggtcgggcgggtgccgcccgaccgagccctcgccggtgctcccgcaaccccggcagcacgcgcgctga

SEQ ID NO: 23 - DNA, 1137 bp, Actinosynnema pretiosum
atgacccccggtcccgtcctccctcctcggcttccctccctcaccggcatccgcgcgccgctggccctgctcgtgttcgtggcccacgcgctcggctccgcccggttcttcgccgacgaatcggtcaactcgctcgggttcctcctgccctacgggcccgcggcgctgtcgttgttcttcgtgctcagcgggttcgtgctggtgtggtcggagccgtggcgcgagggcgtgggcccctacttccgacgccgggtcgtgcgcatcctgcccacccacgtgctgacgtgggccgcggtgctgctgctcctggcggcgctcggcccgctgccgctgctggggccgctgcccgaggtggggcccgcgctggtcaacctgtcgctgctccagtcgctcgtgccgctgcccgactacctgctgtcggtcaacggcatcaactggtcggtgtcctgcgaggtggtgttctacctgctgctgccgctgctgtcccggccgctgctgcgggtgcccgaccaccggttgtgggcgtgcttcggcgtgctcgccgcggtggtgctcgcgctgccgggcgtgatcggggcgctggtggacgggccgccgtgggcgctgtggccgccgctgtcgttcgagcaggcgtggctggtgaacttcttcccgctgacccggttgccggagttcctgatgggcgtggtgctcgccaggatcgtggccaccgggcggtggcggccggtgcgggcgtggtggccgctgctgggggtggcggcggtgtgggcgctgctgccggtgctgccgcaggtgtacgcgcgcagcgcgatcgcggcggtcccgctggcgctgctcgtccccgtcatcgccgtgcgggacctggaggggcggcggtccgtgctgtcccggcggtgggtgcgggtcgcgggggacatgagctacgcgacctacctgctgcactggccgctgctcgcggtcgccaagcacgtggcgggggaccggctgctcgggctgggggaggggctgctgctggtggcggcgctgtacgcgctcacccaggggctcagcctgctgctgcaccggtgggtggagcgtccgctgctgcggcgcgcccaccggccgaggcggtcgcccgccccggacccggcggggtga

SEQ ID NO: 24 - DNA, 348 bp, Actinosynnema pretiosum
gtgaagtacatgctgctcatccacaccagcgacgagcggtggcagaccctgacgcaggccgacaaggaggagttcctggccgagtacgccgcgttcaccgaggagatcgagtccaccggcgagcactgccacggctccccgctgacccaccgcaggaacgcgctgtccgtgcgggtgcgcgacggccgcgccgccgtggagcgcggcccgctcaccgacgacgaccgctacctggccgggtacttcgtggtggactgcgccgacgaggaccgcgcgctggagatcgccgcgcgcgtgcccgacgcgaagctgaacgtggtggaggtcagggcgctcgcggtggtctga

SEQ ID NO: 25 - DNA, 2007 bp, Actinosynnema pretiosum
gtgctcggcctgaacggcaacttctcgaccgccgccgaggagctggtgccggggatgctggagttcttcttccacgactcgtcggcgagcctggcgcgcgacggcgagctgctggccgccgtggaggaggagcggtacaaccggatcaagaagaccacccggttccccagcgcggccgtgcgggcctgcttcgaggagaccgggctggggcccgacgacgtggacgcggtggcgttctacttccccgaggaccacgtggacaaggtgctcggccagctctactgccagagccccgccgtgccgctgcggtactcgcgcgacctggtgcgcgaccggctgcgcagcgagctgggctgggacctgcccgcggagaagctgcactacgtgccgcaccacctggcgcacgcgatgtcctccttcgggcactccgggttcgagtcggcgctgacggtcgtgttcgacggccggggggagaacgcgtccgggaccgtctaccggatcgggcccggcggggtcgagtcgctggcctcgtaccccgtgcaccggtcgctggggttctggtacctgttcggcacgcagctgctcggctacggcttcggcgacgagtacaaggtgatggggctggcgccctacggcgacccggcccgctaccggtcggtgttcgagtcgctgcactcgctgggcgagggcggcgagtacgagctgctgggcaacgtggtcacgcccaacgccttcctccccgcgatgctcgacaccgggttcgcgccgcgccgcaagggcgaggagttcacccaggaccacaaggacttcgccgccgcgctgcaggagaccgtggagcggatcgcgctgcacgtgatccggcactgggccgagcggaccggtgagcgcgcgctgtgcttcggcggcggggtcgcgcacaactccagcctcaacggggtgatcctgcggtcggggctgttcgaccgggtgttcgtgcaccccgcctcgcacgacgcgggcgccgggaccggggccgccctggcggtggagcaccggctgaccgggcgggtgccgcgcgtggcgctgcgcagggcggacctcggtggtgggctcggggacgagcgggacgtcgtgctcaagctcaagggctgggacgggttcgtcgagctggtgcaccgggcggcggacctggaggacctggtggacacctcggcccggctcctcgcggacggcgcggtgctcgggtgggcgcgcgggaggtcggagttcgggccgcgcgcgctgggcagccgcagcatcgtcgccgatccccggcccgccgcgaaccagaccaggatcaacgcgatggtgaagaagcgggagagctaccggccgttcgcgcccgtggtcacccccgacgccgccgccgagtacttcgagatcccggacgtcgaggccgagctggggttcatgtcgttcgtggtgcccgtgcggcccgagcaccgggccgggttgggcgcggtgacccacgtggacggcaccgcgcgggtgcaggtggtgacctcgacggccaacccggcgttccaccggctggtggagcggttcggcgagctgaccggcacgccggtgctgctgaacacctcgttcaacaaccacgccgagccgatcgtgcagacctttgaggacgtgctgaccagcttcctgaccaccgagctggactacctggtggtcggcgactcggtggtccgcaggcgcccggacttcctgggcgcgctggacgacgcggtgatccggttccgccccgagaccaggctggtgggccgggtgcggttcccggtcggtggcggtcgggtggaggagcacgaggtcttcctggactacgcggaggggccgcgcaaggcgatctcggcgcgggcgcacgggctgctgacgcgggtggacgggaagagcgcggtggccgaactggtggacggcgaggtggacgacgccctccgacaggaggtcctcgacctgtggaccgaccggtacttcacgctggggcctgtttag

SEQ ID NO: 26 - DNA, 756 bp, Actinosynnema pretiosum
ttgctcgttcgggcggtcgagcgggctggtgggggtgggggtgtcaggtcggttggtgtcgctgtgcctgccacgctcgatcggtccggcgtcgtggtcgcctggccgggtcggcccgggtgggtcggggtggacgtgctcggtgcgttgggggcgatgttccccggcgcctcggtgcggcacggggatgacggtgacctcgcggcgcttgccgaggccgatgccgccgggtgcggtgagctgctgtacgtcggtgtgggcaccggggttggtggcgggatcgtctcgggtggactgccgctgaactcgggtggctcgtgcgaggtggggcacgtggtcgtcgaacggggtggggggctgtgcgactgcgggcggcgggggtgcgtgcaggcgttcgcgtccgggcccgcgattctgcgcggggccgtcgcgcgtggtgggcggcccggtgggctgggggacgtccgggacggggtcgcgcgtggggagcggtgggcggtggaggcttgtgaggacgccgcgtccgcgttggccgcgctggtgatcggggtgggggagctggttcggcccgagttggcgctgatcggcggtggtgtcaccaccgcgctgccagggctcgtgcccgctgtggccgagcgggtcgccgggctgggcaggaccgggcacccggtgccatcggtgcgaccggcggtgctcggggccgagtcctcgctgcacggggcgcttgtcctggcgcgtgggggaaaccgggagtag

SEQ ID NO: 27 - DNA, 435 bp, Actinosynnema pretiosum
gtgcccgacctgctcctgctgaacggcccgaacctgggaatcctgggccgccgcgaacccgaggtgtacgggaccgcgacgctcgccgacatcgaggacgcggtggccgacgaggtggcggaccgggggtggaaggtggtggcgaggcagcacgacggcgaagccgagatgatcgggaccatccaggacagctacgacacggtgggcgctatcgtgaaccccggcgcgctcatgatcgccggctggagcctgcgggacgccctggcgaactacccgagcccgtggatcgaggtccacctgtcgaacgtgtgggcccgcgagccgttccgccaccagtccgtgatcgccccgctggcgacgggcctgatcgcgggcgtgggcgccctgggctaccgcctggcggcacgggcgctgctggaccgggtcgggggttga

SEQ ID NO: 28 - DNA, 1167 bp, Actinosynnema pretiosum
gtgcacctgaaggccccgcagaagctggagttccccgcctggccgcagttcgactcgaccgagcgcgaggccctgctgcgcacgctcgacagcggccggtggtggcggatcaccggggacgaggtcgtctcgttcgagcgggagttcgcgcaggcccacggcgcccggcacgcgctcgccgtcaccacgggcacgcacgccctcgaactcgtcctggaggtcatgggcgccgggcctggcaccgaggtgatcgtgcccgcgttcaccttcatctcctcctcgcaggccgcccagcggctcggcgcggtcgtcgtccccgtcgacgtcgacccggacacctactgcgtcgacccggccgccgtgcgggccgcgatcaccgagcgcaccagggcgatcatgcccgtgcacatggcgggccagctcgccgacatggacgcgctgcgcgccctgtccgccgagaccggcgtcccgctgctccaggacgccgcgcacgcccacggcgcgcggtggcgcggcgagcccctcggcgcgctcggctcgatcgccgccttcagcttccagaacggcaagctgatgaccgcgggcgagggcggcgccgtgctgctcccggacggggaccagcacgaggaggcgttcctgcgccacgcctgcgggcgcccgctcgccgaccgggcctaccaccaccgcacggccgggtcgaacttccgcatgaacgagttctccgccgcgctgctgcgcgcccagctcggcaggctccagcagcagaccgacgtgcgcgagcggcactggcccgtgctgtcgcggctgctcgccgaggtgcccggcgtcgtcccgcaggggaccgacccgcgctgcgaccgcaacccgcactacatggccatgttccaggtccccggcatcggcgaggagcgccgcgcccgcctggtcgacgcgctggtcgagcgcggagtcccggcgttcgccgggttccgcgcgatctaccgcaccgccgcgttctgggagcgcgccgcccccgacaccacggtggacgccctcgcccgcgcctgcccggtcacggaagcgctcagcagcgactgcgtgtggctgcaccaccgggtgctgctgggggcggaggagcagatgccgctggtggcggaggccgtcgcctgcgcgctggaggaggcctga

SEQ ID NO: 29 - DNA, 1209 bp, Actinosynnema pretiosum
ttgcgcgttctgttcaccgtcaccggggccccgtcgcacgcgcacgagatgctcgtgctcgggcgcgcggtcctggacgccgggcacgaggtgctcgtggccacggcccccagcgccgcgcccggcctgcggggcagcggcctgccctcggtcgaggcgttgcccgaccaggcgaggttctgggtggagcggctcgggggcgtcgcgccaccggccaggccgggcacggactaccccggcgtcgccgcgatcgtcgacgtcatcgcgggcaggccgctgctgcgggaggcgctgaccgggctgcgccccgtcgtcgaccgcttccggcccgacctggtcctgcgcgacggcatggagctggccggcctgctcgtcgcggaggagcgcgggctgccctgcctgaccgtgccctcgggcacgacgaaccagctggacccggccgccgtgctgccccggctcaacgagcgccgcgccgagcagggcctgcccacctcggccgacccgctcggcgcgcaccggcacggccacgtcgactgcctgccgcgcgagtactcgctgctcggcgtcccgctcagcccgtgcctggcctaccggccgccgtcgcccgacggccggtcgctgcccgcgtgggtggccgacctggacccgtcccgcccgctggtgtacggggcggtcggcatcgcgctggccgccctgtggcgccgcgacgacgtgaccgcgctggtcgccgacccggtggaggcgctgggcggactcgtggcggccctgtccgaggtggactgcgccgcggtggtgtccaccgggggcatcccggtcgaccgcgtcccgccgggcgcgccgcacgtgcgcgtggtcgagcgggcgccccagccgctgctgctggagtgcgcgcagctgttcgtcacccacgccggggccaacagcgtgcgcgaggcggtgcgggcgggggtgcccatggtcgccgccccgctgttcggcgaccagccgcacaacgccgccagggtcgccgcgctggggctgggcgtgcgggtggtggacggctcgcccgccgatctggcggacgcctgccggaccgtgctggccgatccccgcttcaccgcgagggcgcgccacgcgcgccggaccctgctcgcgctgcccccggtgggcgaggtggtgacccgcctggagcagatcgccgacgccgcgcggagcgtcgcggtggtgtga

SEQ ID NO: 30 - DNA, 243 bp, Actinosynnema pretiosum
atggccaccggtgagaccggcttcgacgacgtcagcttcgacctgatctcggtgcagtaccactccctcaaggcggggcacgactacggccagtacgtccgcgacgccgacaacgcgggccgccaggacatcgcggacttcttccgccgcgtcatggacgaggacgccgcgcgcgccaaggagtgccaccggttcctggccgagctgaccggcagcagcgagtccggcccggcgatgagctga

SEQ ID NO: 31 - DNA, 1026 bp, Actinosynnema pretiosum
atgcccaggaggttggcggagcaggtcgtcgtcgtggtgggcgcgtcgtcggggatcgggcgggtcaccgcgctgcggttcgcggcggcgggggcgcgcgtggtgtgcgcggcgcgcaacacccgcgccctggacgggctggtcgaggaggtccggggcgcgggtgggcgggcggtcgcggtgacggcggacatcgccgacgaggcggccgtgcgcgcggtcgccgacgtggcggtggagcggttcgggcgggtggacacctgggtcaacgcggccgggatcggggtgtacgggcgggtggaggacacgcccgccggggagttcgaccgggtgatgcgggtcaactacctcgggcacgtgcacggggccaaggccgcgctgcccgcgctgcgccgggcgggcgggggagtgctgatcggggtcgcgtccgtgctcggcctgcgctcggctccgctccaggccccgtacgcggccagcaaggcggcggtgcgggcgttctacgacgcgctgcgggtggagctggcccacgacggcgagtcgatcgcggtcaccgccgtgcttcccgccgcgatcaacagcccgttcttcgagcactgccgcagccgcgtcggctcgctgcccaagccgccgcccccggtgtacgcgccggagctggtcgccgaggccgtgctgcgcgccgccgagcgcccccgccgcgaggtgccggtgggcgacgctgcgctcgcgttctacctggggcagcggctgttcccggcgctcaccgacgcgctgatgtcggtgcgcgcggtcggccggtcggggatgcggtccgagctgccggacaacggggtggacaacgtggactcgccggtggacgaggacgggcgggtgcacgggtcctaccccggccgggtgctggactccagcccggtgacggcgctgctggcccgcgtgcccaggccgggggagctggcgacctcgctggtgagcggggcgcaccgggtgggcggggcggtgcggggcgggctgcgcgcggcccggtcctga

SEQ ID NO: 32 - DNA, 465 bp, Actinosynnema pretiosum
atgaccgacacgacgacgcgccaccgcacgccggtgcagaacgccgctttagcggtgggcgcggtgttcctgctggtgggcatcgccgggttcgtccccggcctgaccacgaactacgacggcctggagttcgcgggccaccactcggacgccgccctgctgggcgtgttcaaggtctccgcgctgcacaacctggtgcacctcgcgttcggcgcggcgggcatcgccgcggggctgtccaaggccccgcgcggggcccgcgcgttcctgatcggcggcggcgcgatctacgcggtgctgtggctgtacggcgtggtcgtggacgagcacagcacggccaacttcgtgccgctcaacgccgccgacaactggctgcacctcggcctggccgtcggcatgatcggcctcggcgcggcgctgggccgcaacgcggcgcactaccgcgcggcgcggtccgacgactga

SEQ ID NO: 33 - DNA, 582 bp, Actinosynnema pretiosum
gtgagcgcggccgtgcggcgcgagcagctcgtggccgccgcgctgcgcgtgatgaagcgcgacgggatcgccgcggcgaccacgcgcgcgatctgcgccgaggccgacatgccgcacggcgcgttccactactgcttccgctccaagcaggagctgtacaccgcgctgctcaccacgggcctgaacaccagcctggacgcggcgtggcccgcgatctcgccggacctgcccgccgtggacaacgtccgcacgatgctggccgccttctggaccgcgttccaggccgacccggaggcggagctggtgctgggcgagctggtcaccctcgcccagcgggagcccgagctgcgcgagctgcccgcgtggcagcaccgcgagtacgtcgccaaggcggtcgcgcacctggaccggctcgccgaggaggccgggttcgactactcggtggaccgggcgctgctggcggagctggtggtgtcggtgctggacggggtcgcctcgtcctggttctcgcaccgggacggggagctggcgaaggcgtcgctggaccagttcgccgtgatgttcgcggccctgacgcgctga

SEQ ID NO: 34 - DNA, 3018 bp, Actinosynnema pretiosum
gtggtcgccaccggcacgcgcatccccggccccaagcccctgcccctggtggggaacctgctcgacgtcctcacctccgacctggacaccgacgtcgacttcctcgaccgctgccaccgcgagcacggcggcatcgtcgcgctgaccttcgccgggcagcgccaggtcttcgcctccagccacgagctcgtcgcccgcatgtgctccgacccgagctggggcaaggccgtgcaccccgcgctggagcaggtccgcgacttcgcgggcgacgggctgttcaccgcgcgcggcgacgagccgaactggggcaaggcgcaccggctgctcatgcccgccttcggccccaccgccatgagggaccacttcccggccatgctcgacatcgccgagcagatgctcgtgcgctggcggcggttcggccccgaccaccgcatcgacgtcgccgacgacatgacccggctgaccctggacaccatcgcgctgtgcgccttcggcgcccggttcaactccttctaccgcgaccgggcccaccccttcgtcgacgcgatggtccgcagcctcgtggaggcgggcgagcgcgccgagcggctgccgggcgtgcagccgttcctggtgggcaggaaccagcggtaccgcgacgacatcgccaccatgaaccggatcgccgacgggatcgtcgccgcgcgcgccgcactgcccgcaggggagcggccggacgacctgctggagcggatgctcacctgcgccgacccggtcaccggggagcggctgtccgcgcggaacgtgcgctaccagctggccacgttcctgatcgccgggcacgagaccacctccggcctgctctccttcgccgtgcaccggctgctggcgcaccccgaggtgctgcgcaaggccaaggacgcggtggacggggtcctgggcgaccgcgtgcccgcgttcgaggacctggcccggctcgactacctcgggcaggtgctgcgcgagaccctgcggctgcacccgaccgcgcccgcgttcgccctggcccccgacgagcccgcagagctgggcggccacgcgatcggcgcgggcgagccggtgctggtgatgctgccgacgctgcaccgggacccggcggtgtggcgcgaccccgacgtgttcgacccggagcggttcgcgcccgagcggatggacgagatcccggcctgcgcgtggatgccgttcgggcacggggcgcgggcgtgcatcgggcggccgttcgccctgcaggaggcgaccctcgtgctggcgctggtgctccagcggttcgacctcgcgctcgccgaccccgaccaccggctgacgatcaagcagacgttgaccctcaagccggactcgctggtggtccgcgcccggccgcgcgccgaccgccctggcgccaccgcgaccgtcgaaaccgttgtgccgcaccaggttccggccacccatcggcacggaaccccgctgcacgtgttctacgggtcgaacggcggcagcggcgagggcctggcgcgcacgatcgccggtgacggtgcggctcgcgggtgggcgacttccgtcgcgccccttgacgacgcggtgcgcgccctgcccgcgtcgggaccggtggtgatcgtcagctcctcctacaacggcgccccgccggacaacgcggcccacttcgtgcggtggctcacgcaggacgggcccgacctgtccggagtggactacctggtgctcggctgcggcaacctggactggtcggccacctaccagcgcgtgcccaccctgatcgacgaggccatggcggcggcgggcgcccggcggctgcgcgagcgcggggccaccgacgcccgcgcggacttcttcggcgactgggagcgctggtacgagccgctgtggccgctgctgtcggccgagtgcggggtcgaggtgggcgagatcgggccgcgcttccgggtggtcgagtcggacgcggccgacggcctcggcgatctcgcgtcggcggtcgtgctggagaaccgggagctggtgcgcggaccggacgccgggtccaagcggcacctggagctgcggctgcccgacggcacgtcctaccgcaccggcgactacctgtccgtgctgccgcagaaccaccccgacctggtgcggcgggcggtggcgcggctgggcacgcgcgcggagcgggtcgtgacggtcgagtcgagcgcgcccaccgggctggtgccggtcgggcgcgcgctgcgcgtggacgagctgctgacccggtgcgtcgacctgtccgcgcccgcgggcgccggggtcgtcgcgcgcctggccgagcgctgcccctgcccgccggagcgcgccgagctggccgccacgaccggggcgacgctgctggagctgctggagcgcttcccctcgtgcgcggtcgacctggcgctggccctggaactgctgcccgcgcccaggacccgcctgtactcgatcagctcggcggccgaggagcagcgggccgaggtggcgctcaccgtgtcggtcaccggcgtgacctccggctacctgagccgggtgcgacccggcgaccgcgtggccgtcgggatcgcgtcaccgccggagtcgttccgcccgcccgccgacaacaccgtccccgtggtgctgatcgccgcgggcaccgggatcgcgccgttccgggggttcctgcgggcgcgggccgcgctcggcggcgaaccgggcccggcgctgctgctgttcggctgccgggggcccgagctggacgacctgtacgcggaggagttcgccgcgctcggcgactggctggaggtggaccgggcctactcgcgccaccccgatggcgaggtccgccacgtgcagcaccggctgtggcagcggcgggaccgggtgcgcgagctggtggacgccggggcgcgcgtgtacctctgcggcgacgcgacgcgggtgggacccgcggtggaggaggtgctggggcgcatcggtcccggcgccgggtggctggacgcgctccgcgccggggggcggtacgcgacggacgtgttctga

SEQ ID NO: 35 - DNA, 1047 bp, Actinosynnema pretiosum
ttgtggattggctattccgcggacggccgtcggcgccgcccggtccggtggtggggcgtgaccgaggccttcacgtgggagaccgacgaggacatcaagctggcggccacgctgcgccgcgacgcggaggcggcgaacgcggccgactccacccgcgcctacctcaaggcgatcggccgggtgcggctgctgaccgccgaccaggaggtggagctggccaggcgggtggaggcgggcctgtacgccgagcacctgctggacctgccgcccgcggcgcgcgggtcgcggcggccggtcgagctgcggcgggtggtgcgggacgggcagcgcgccaaggaccacctgctggaggcgaacctgcggctggtggtgagcatcgccaagcggtacaccggcaagggcatggcgctgctcgacgtcatccaggagggcaacctgggcctggtgcgcgcggtcgagaagttcgactacgccaaggggttcaagttctccacctacgccacgtggtggatccggcaggcgatcacgcgcgcgctggccgaccagtcgcgcaccatccggctgcccgtgcacatggtggaggtggtcaccaagctggcgcggatcgagcgggacctgctgcaccgcaacgggcgcgaggcgaccgccgaggaggtcgcggtcgagctggacgtgacgccggagaaggtcgtggagctgcggcagcacgcgcgcacgccggtgtcgctggaccagaccatcggcgcggaggcggacacctccatcggcgacctgatcgcggacgagaacgcggtggccccgctggaggcggtcagctcggcgctgctgtcggcggacctgcgggcggtgctggagacgctgacgccgcgcgaggcgggcgtggtgcggatgcggtacggcttcccggacggggtgctgcacacgctggacgaggttgggcgggtgtacggggtgacgcgggagcgcatccggcagatcgagtcgcgcaccatggcgaagttgcagcacccggcgcgctcgcgcgggctggagggctacctggactga

SEQ ID NO: 36 - DNA, 204 bp, Actinosynnema pretiosum
gtgtggatcaccgagcagaacgccaggattccggcgaggcggtcgtcgggcaggtcgaacgcgcgcaggtgcgcggtgaggtggccgaggccgcagccgaggtccgccacgcgggcgccggggcccaggagctcggcgaacgcggacagcagggcgcgttcgagggggagggacccgaggacgtcgctgaacaggtccgcgtag

SEQ ID NO: 37 - DNA, 573 bp, Actinosynnema pretiosum
gtgcgcaagctcgtgtacttcatcgcggcgagcatcgacggcgtgatcgcgggcccggagggcgagcacgacttctacccgaccgcagagccgatggtcgagcacctgcgcgccgagtaccccgagaccacgcccacccacgtccgcccgctcgtcgggatgcccctggacaccccgaaccgacgctacgacaccgtgctcatgggccggggcagctaccagcccgcgctcgacgcgggcgtgggcagcccgtacgcgcacctgcgccagtacgtggtctcccgctccctgcccgacgccgatccggccgtcgcgctggtgcgcgcggacccggtcggcctggtgcgggagctcaagcgggaggacggcctggacgtgtggctgtgcggcggcgcggacctggccgggcagctgctcccggagatcgacgagctggtggtcaagacctacccggtggtcgcgggcggcgggaccccgctgttccgcgccggtttcgccccgcgcgcgttcaggccggtcgacgtgcgcgcgttcgaccacggcggcgtggtcaccacctaccgcccggcctga

SEQ ID NO: 38 - DNA, 615 bp, Actinosynnema pretiosum
atggcgcgacgcaacccggagcggcgggccgcgctgctcgacgccgccgtcgaggtgctggcgggcgagggcgcgcgggggctcaccttccgggcggtcgaccagcgggccggggtgcccgcgggcaccgcgtcgaactacttcgccgggcgcgccgagatcctcaccggcgcgggcgagcgggtctaccagcggctgctcgaccaggccccgctgggcgagaccccggacggcccgcgcgaccggtcccgcgtcaccgaattgatgcacgccctcgtcgaccgcgtctcggccttcccgaccggtttcctcgccctgctggaactgcgcctggaggccgcgcgcaacccggagctgcgcgaggtcctgaccgcccgcgtccgcgccgacctgcgcttcaacgccgagcaccacgccgcctccggcctgcccggcgacggcaccaccgtggcgctgctgtggctggccctcaactggctgatcctggaacgcctcaccctccccgacctgctgaccgaccaccagcgccgcgacctcgtcaccgcactggtcgaccggctgctcgcggggcagccggaggaggcgggcagcccgttcgacgagagcggaaaatga

SEQ ID NO: 39 - DNA, 1179 bp, Actinosynnema pretiosum
ttggcagtgggaaaggccgacagcgcggtgggcgggtggctgcccgccctgctcatcgtcgcgctcaccggcacgatcatcgagatcgacatgtccgtgcccagcttcccggacatcgcccgcgaactgggcgtgggcggcccggccgtgcagctcaccgtcaccctcaacttcctcggctactgcctgggcgcgctgtgccacggcccgctctccgaccggttcggccgccgcccggtgctgctcggcggctccgccgtcatgctggtcggggcgctgggctgcgcgctcgcgccgacgatcgggctgctgctgaccgcccggttcgtccagggcatcggggccagcgccgccgcggtgctggtgttcgtgatcatcggcgaccgctaccggggcccggcggcgttgcggatgtacggcttggtgaacgcggccatgtccgcgctgatgacctgcgcgccgctcctgggcggggcgatcaaccgggtggccgggtggcgcggcaactacctcgccgtgtgcgccaccaccgcgctgtccctgctgctggtgttcctgttcctgcccgagacgcgcacccgcgcgcgcaccgcgatcaccgcccggtcggtgctcgccgactaccggaggctgctcgggagccgcgcctacctggccgcgtcgctgacgcccagcctgctgttcgccgcctacctggtgttcatcgcggccagcccgttcctctacaccgccgcgttcggcctcggcgtcaccgggttcgcgctgcacctgctcgtcgtggtcgcctcgttcgcggtccccagcgcgttcgccaccgggatcatcccgctgctcggcggcccggcgggggcggtgcggtggggcctgggggcggtcgtggtcggcgtgctggggttcctgctgctcggcgacggcccggtcgccgtgaccggctcggtcgcgctgttctgcgtcggcttcgccgtgtgctacccggtggtgttcgaccggtccatgtcggtgttccccgagctggcgggcgcggcgtcctcggcgaccatgggcctgcgggcgctcctggtgaccctgctgaccgccctcgctggcgccctgtccacgggcgggccgttcgcccccgccgcggtgatggcgggggctgtggccgcggtggtgctgctgagcgcgctcagcccccgctga

SEQ ID NO: 40 - DNA, 318 bp, Actinosynnema pretiosum
atggttaccgcaagtgcggtcctgtgcgcccgcgcgggctgcggcgcgccccgtcccgcaggcgccacggcccgctggcgcttctgctccaccgcctgcaagcaggccgcctaccgcctgcgccgcaaagcgggcagcgcctcccacgagcacgaggacgagcagctccgcgaggcgctgctcgccctcgtggcacgggcgcggaccctgaccgaggccaccccgccctggtccgcgcaggccaccgcgctggtcgggctggcccggcgggtggtggagctggcggcggcgcgggacgccggggtcagcgggggctga

SEQ ID NO: 41 - DNA, 1677 bp, Actinosynnema pretiosum
atcgtgccgggtctggtggtggccctcgccgccaccggcgtcctcgcggcccccgcgagcgccgccatcaccagccccaccgtgaccagcaccgccaccaccgtcaccttcgaggtcggccgcaccgggacgcccgcgttctcgcgcgtctacgtcgacaccgaccgcagcgcggccaccggctacgcgcagggcgggatcggggcggaccacctgctggagaacggttccctctaccgccacagcggcggcggctgggcgtggacgcaggtgcggaccgtgccgttcacctccgcctcgggcgtcgcgcggtggaccgtcgagcgggccgacctcggcgagcaggcgagcccgaacgacgtcgacgtggtgttccaggtcgaggccccggtggagacctcggcgaaggtcacccacacctacaccgcgggcgcgggcggctcggtcgcctaccagccgtcgagcgcgaacgtccccaacccggaacgcggtctttaccaccacaccggcgactgcgacaaggccgacctgtccgccgacgcgctgcgccggtaccgcaccgagcagggcatcaccctcgcgatgtgcgtgttctacctggccgagcaccgctcgtccccgatcgcccagaccgccctcgaccgcctgcagcgccagttcgacgccgcgcgggtcgcgggcgtgaagctggtgctgcgcttcgcctacaccgcgtcggagtccggcgaggacgcgccgaaggagcgggtcctggcgcacatcgaccagctcgcgccgctgctgcgcgcgaacgcggacgtgaccgccgtggtgcaggccgggttcgtcggcgcgtggggcgagtggtactacaccaagaacttcggcaacgcgggcgtcgtctccgcgaccgaccaggccaaccgcaaggcggtggtggacaagctcctgaccgcgctcccggcggaccgggcggtgcagctgcgcacgccgaagttcaagcgcgccctgtacgggggcggcagcaccaggatcggccaccacaacgactgcttcctggccagcgacacggacttcggcacctacgaggacccggcgaccgaatacccgtacctggaggcggagacgcggacgctgccgatgggcggcgagacctgcgcggtgaacccgccccgcaccgactgcccgaccgcgaccgccgagctgtcccgcttccactggacgtacctgaacaccgactaccagcaggacgtgctgcgcgcgtggtcggcgcagggctgcctggcggaggtcacggccaagctcggctaccgcttcaccctgcgcgactcggtgttccccacgacgaccaccaggggcgccgcgctgcccgtgcgggtgaacctgaccaacgacggctacgccgcggcggtgaaccagcgcaccgtgaacctggtgctgcgcaacaccaccaccggcgccctgacccgcctcccgctcaccgcggacccccggacctggctgccgggcgcccgcgccatcgcgcaggacgcccagatcccgccgtcgctcgcgacggggcagtacgcgctcctgctggaactggccgaccccgccctgccgggccgcccggagtactcgatccagacggcgaacaccggcctgtgggacgcgaggacggggctgaacgacctgaagcagaccatcacggtgtcgtaa

SEQ ID NO: 42 - DNA, 1314 bp, Actinosynnema pretiosum
atgacactcgcggagttgctgcccagcgtcggcatgaccggcgaaccggccctggagacggggctgtggcccctcgggacccggctggagcggggggagctgctgctcggcggggtgcccgcgaccgagctcgccgcccggttcggcacgccctgccaggtgctcgacgagggcacggtccgggcgcgggcgcgcgcgttccgggaggcgctgccggaggcggaggtggtgttcgcgggcaaggcgctgccgtgccgcgaggtgtaccggtgggtggcggacgaggggctgtcgctggacgtgtgctcggccggggagctggcgatcgcgcggtcggtcgggttcccggcggagcgcgtcctgctgcacggcaacgtcaagacccctgaggacctcaaggccgcgctcgggtacggggtcgggcgggtggtggtggactcgttcgacgagatcgagcagctcggggcgctggccgaggggccgcaggacgtgctggtccgggtcacccccggcgtcgacccgcgcacccaccgggcggtggcgaccggggtggaggaccagaagttcggcttctcgctcgcgggcggggacgcgctggaggcggtgctgcgggtggtcgagcagccgtcgctgcgcctggtcgggctgcactgccacgtcgggtcgcaggtgcggcacgtcgcggtgtacgaggaggctgcgcggcgggtggtcgggctgatcgcctcgtgcggggtgcggatcgagcagctggacctcggcggcgggttcgcggtgccgtacctgccgggggagggggagttcgacctgggcgggttcgcgcaccgggtgcgggtggcgctgagccacgagtgcgcgctgcggcgcgtgccggtgccgaggttgtcgatcgagccggggcgggcggtcgtggcgcgggcgggcgtgacgctgtaccgggtggcggcggtcaagcgcggggtgaggcgggtgttcgtggccgtggacggcggcatgagcgacaaccccaggcccgcgctgtacgggagccggtacgcggtgcgcctggtccgccgcggcgggcggcgcgcgccggtgacggtggtgggcaggcactgcgaggcgggcgacgtgctcgccgaggacgtgccgctgcccgaggacgtgcgcgccggggacctgctggcggtgccggtgaccggtgcttaccaccacgcgctggcgtcgaactacaacgccgtgggcaggccgccggtggtgggggtgcgcgacggcgtcgcgcgggtgctggtgcggcgggagacggaggaggacctgctgcggcgggaggtgtga

SEQ ID NO: 43 - DNA, 435 bp, Actinosynnema pretiosum
atggacgaggtggacgacccgaccacccagcccgcccccgagcccggcacaccggcaccggccacgctggacctcacggcccgcccggccctggccgaggtgcgccgctggaccggcgcgctcctggccgacgcggacgaggagtcggcggacgacgtgctgctcgtggtcaacgagctggtcgccaacgcctacgaccacaccacctccccactcgccctgcgcgtcacgaccaccccggaccacgtgcgcgtggaggtcgaggacggctcccccgacccgccgcgcccggacctcaccgcgggcctgcgccagatcggcacgcgcgggcgcggcctgctgctgatccgccagctgaccgaccgctggggcagcgcgccccaccccggcggcaagaccgtgtgggcggagctgccgaacgtccccgcgccctga

SEQ ID NO: 44 - DNA, 387 bp, Actinosynnema pretiosum
gtgtccgagctggatgcagccgcggtggtcacggtggcttccgacgtggtgcgcggggtgcccgtgctgcgcgtcgccggggagatcgacgccaacgtcgcggacgaggtccgccgggcgctgctgccctggttggacgggttgcgcgggccgggggtgctcgacctgaccggggtgcggttcatggcctccacggggttgtcgctgctggtcgaggcggctcggcgcggccgggcgcggctggtgctggccaccgcgcagcgcggcgtgctccggccgctgcagctgaccgggatgagcgcgctgctccccacgcaccccaccgtggacctggccgtcgacgcccagcgcggggccgacctgaccaggatgcccagcacggcctga

SEQ ID NO: 45 - DNA, 573 bp, Actinosynnema pretiosum
gtggtgcgcagtcccgtgcgcaggagagcggtcgtggtggtgtccggggtcgtggtggtggccgcgctcgtggggttgtgggtgttccagccgtggcggttgtggacgcgcagcgaggcggacgaggcgctgcccgccgagttcaccgcctcgaccagcgcggggccgagcaccggggcggcgagcagcggcgccccgacgcccagcgccccgacgtcggcgcccgtcgaccagcccgcggtgctggccacgggcgggttcgtcacccaggagcacgagacgcggggcaccgccaccgtgctgcgcaacggggccgcgcgcgtgctgcggctggaggggttctccacctcggacgggcccgacctgcacatctggctctccgacgcgaccgcgggcggcgagtggggcaagtacgacgacgggcgctacctgccgctcggccccatgaaggccaccgacggcaaccagaactacccgatcccggaggacgcggacctgtcggggctgcgcagcgtcgtggtgtggtgcgaccggttcaacgtggccttcgggtcggcgccgctggacctgtga

SEQ ID NO: 46 - DNA, 1011 bp, Actinosynnema pretiosum
atgggggctctgatcgtcgcggtggtgctgctcgtcgtcttcctggtgcagctcaagcgggagcccagacgactgggcaacggcgtgtacctgctgatgagcctggcgttcctcgcgctctggctgctcaccctggccgaaccgcagaccagggtgctggtggtcggcgccgtggtgctgatcgccccggtgttcgtcgtcgtgatcgcgctcttcctgatcgccaacggcgtgaccctgctgcggcgcgagggtttcaagccgggcaacgccctctccttcggcgcgggcacggccatcctgggcgtggtgggcggcctgctcctggtcctgctgtccgcgctgcgcgagggctcccccgagccgtgggtgctggcggcggccggttcgctgttcctgctcgcgggctacctcggcttcgccttcaccctcttcctgctctactcagtgctctacggccgcgtccgcaagcgcaccggccacaccgccatcgtcgtcctgggcgcgggcgtccccggaggccgcgtcaccccgctcctcgcgggccgcctggaccgcgcgctgaagctctaccgccgcgccgcggccaagggcgcttcccccctggtggtcgcctccggcggccagggccccgacgagcccgcctccgaggccgaggtcatggcgaactacctgcgcgagcgcggcatcccggacgaggcgctcctggaggagcgcgagtccacctccacctgggagaacctccgcctctcctccgccctgctcgccgagcgcggcgtcaccggcaggctcctcgtggtcaccagcagctaccacgtcccccgagccgcgatcctgtcccgccgcgcgggcctgaaggcggacgtccggggcggtcgcaccgcctggtacttcgtcccgaacgccttcctccgcgagttcgccgcgctcctggtccagtaccgcgccctcaacgccctggccgcctgcaccgcgctgtccgtcttcccgctgctggcctacggcgtctga

SEQ ID NO: 47 - DNA, 1167 bp, Actinosynnema pretiosum
atgggcagttctccggacgcaggaatcgacttcccggcctggccgcagcacgacgacgccgagcgcgcggccctgctgcgcgcgctggaccagggccagtggtggcgggtcggcggcagcgaggtcgacgagttcgagcgcgagttcgccgagtaccacggcgcgggccacgcgctcgccgtcaccaacggcacgcacgcgctggagctggccctccaggtgctcgacgtgggccccggcacggaggtgatcgtgcccgccttcacgttcatctcgtcctcgcaggccgtgcagcgcctcggcgcggtcgccgtgcccgtggacgtcgaccccgacacctactgcctggacgtcgccgccgccgaggacgccgtcacctcccgcacctcggcgatcatgcccgtgcacatggcgggccagttcgccgacatggaccggctcgacaagctgtccgcgagcaccggcgtgccggtcgtgcaggacgccgcccacgcgcacggcgcccactggcgcggcaagcgggtcggcgagctcgggtcgatcgccacgttcagcttccagaacggcaagctgatgacggcgggcgagggcggggccgtcctgttcgccgaccaggcccagtgggagaaggcgttcgtcctgcacagctgcggcaggcccaagggcgaccgcggctacttccacctcacctcgggctcgaacttccggatgaacgagttctccgccgccgtgctgcgcgcccagctcggcaggctggactcgcagatcgcgaccaggcaggcccgctggccggtgctgtccgcgctgctggcggggatcgacggcgtcgtgccgcagaccgtcgacccgcgcagcgaccgcaacccgagctacatggcgatgttccggatgcccggcgtcaccgaggagcggcgcaacgcggtggtggacgagctggtgcggcgcgggatcccggccttcatggcgttccgggcggtctaccgcacccaggcgttctgggagaccggcgcgcccgacctgacccccgaggagctggccgcgcggtgcccggtgtcggaggagatcacccgcgactgcgtgtggctgcaccaccgggtgctgctgggcgccgaggagcaggtgcgccggctggccgcggtcgtggccgacgtggtcgcgggcgcgtga

SEQ ID NO: 48 - DNA, 1164 bp, Actinosynnema pretiosum
gtgagcgccgggagcggtggcgtgcgcggtggaccggtgcgggtggcggtggtcgggctcgggtgggtggcgcgggaggtgtggctgccccggttgctgggcggcgccgcctaccgggtggtcgccgtggtcgagccgtccgcgcgggcccgcgccgccgtgctcggcgacctcaccggggtcgccgcgctggagcgggcggaggacctgcgccgcgacgaggtcgacctggcggtggtcgccgtgccgaaccacctgcacgcgccggtcgcgtccggtctgctgcggcggggcgtcccggtgttcctggagaagccgctgtgcctggggtccgccgaggccgccgcgctcaccgccgccgagcgggccggtggcgcggtgctgctgggcgggagcgccgcgcggcaccgggccgacgtgcgggcgctgcgggaggtggccgcgtcgctgggcgcgctgcggcacgtcgacgtgtcctgggtgcggtccaggggcatcccggaccggggcggctggttcaccgaccgcacccgctccggcggcggcgcgctggtcgacctcggctggcacctgctcgacaccgcccgcgtgctgctgcgcggcgccaccggtgacccggagggcgcggagctggagcacgtcaccggcgcggtgtccgcggacttcgtcgacgaccacgcgttccgcgccgcctggcgcggcgacggggcgcaggcggacccggtggtcggcgacgtggaggacaccgccaggggattcctcgtcacccggtcggggatctcgatcggggtgcgcgcggcctgggcctcgcaccaggcgctggacaccaccgtcgtgcgcgtggagggcagcggcggcacggccgagctgcgctgcaccttcggcttcagcccgaaccgcgagggcccgtcgcggctcctgctcaccaccgacggccgcaccaccccggtcgagctgcccgccgagccggtgggcgccgagtacgacgcccagctcgcgagcctgcccgcccgcctggccgaccccgccgcgcgcggcgaggccgcgtccggggcgcgctggatcgcgggcgcgatcgaacgggtctaccgcgccgccgacgccgtgcggtgcgccgaacgacgacacgcgccggtcgacctcgccccgacgggagccccgtga

SEQ ID NO: 49 - DNA, 639 bp, Actinosynnema pretiosum
gtgctgttcgacctcgacggggtgctggtcaacagcttcgccgtcatgcggcaggcgttcgagatcgcctacaccgaggtcgtcggcgacgggcccgcgccgttcgaggagtacaaccggcacctggggcggtacttcccggacatcatgcggatcatgggcctgccgctggaggtggaggggccgttcgtccgcgagagctaccggctcgcgggtgaggtcgaggtgttcgagggcgcgccggagctgctggcggacctgcggcagcacggcttcggcaccgccgtggtcaccggcaagagcggcccgcgcgcccggtcgctgctgaccaccctcggcctggcggagctgttcgaccacgtcatcggctccgacgaggtcgtgaaccccaagcccgcgccggacatgctcctgctggccaccggcctgctcggcgtcccggccgaccgggtggtgatggtgggcgacgccctgaccgacctggcgagcgcccgcgccgcgggctacccggcgctggccgcgctgtggggcgagacggacgaggcggagctgctggcggcgaacccggacgcggcggtccgcaagccctcgcaggtgctcgactggtgcctggcccacctcgccgacgaccggacgtga

SEQ ID NO: 50 - DNA, 795 bp, Actinosynnema pretiosum
atgacgcggaccggttccgcgcaagcgcccctcggtggccccccggcggcgggcgccccgtcctggtccggctggtcgctggccggtggtggcgacgcccgcccggtggccgaggtgctgccccccgtcgtgcgggagctgcgcgcgcgggaccggggcgcgcactggtcgttcctgcgcgggacgggggtggcgctgtggctgggcacgaccgaggccgcgcggggcccggtgctcgccgtgctgcgcgccgaggcggcggcccgcgagctgcgcgtcgacccggcgggcgagggcttgccgggcggtccggtcgagccgctggtcgaggtcgcgacggcctccagcgaactggcgctggacgtcgcggcggcggtgggcccggcgtggggcgaggaccccggcgggtcgggcgcgcaggccacggtgcggcgccacctggccgaactggtcgcgctggccccggaaccggccaggcccgcgttcctgttccactgctggcagagctggggcagaccgctcagcccggccgcgcgcaccgaggccgccgtcgccgcgcagggcccgtcgcccggcgggcgcgtggtcggctcggccggcgcgtggcggcgctacctggaacagatcgatcgcgcgcagctgcgcaccgcggcggaccgccgccgcccggccctgccggtgctgttcgagcaggcgaccctgacccacaaccgcctcggcgtcggcgcggccgtggcggcgcgggcggcgctcgccctgcgcaccgcgctcgtgacgggcgccgcctgcggctga

SEQ ID NO: 51 - DNA, 1029 bp, Actinosynnema pretiosum
gtgcccaccaccgccaccacccgcgaggtccgggtcgagctgggcgagcgcgcctacaccgtccacatcggacacggcgtgcgcgccgccctcccggacgtcgtgcgggccctcggcgcgcgccgcgccgtcgtggtcaccgcccgcccgccggaccagacgcccgaccccggcgtcccctccctcgtcctcccggcccgcgacggcgagcacgacaagaccctcgccaccgtcgcccacctgtgcagccggttcgccgagttcggcctgacccgcgccgacgtcgtcgtctcctgcggcggcggcaccaccaccgacgcggtcggcctcgccgcctcgctctaccaccggggcacaccggtcgtgcacgtgccgacctcgctgctcgcccaggtcgacgcgagcgtcggcggcaagacggcggtcaacctccccgagggcaagaacctcgtcggctcctactggcagcccgccgccgtgctgtgcgacctcgacctgctcgccaccctgcccgagcgcgagctgcgcaacggcctcggcgagatcgcccgctgccacttcatcggcgcgccgggccttgaccggttgccgctgctggaccaggtggcggcgagcgtcgccctcaaggcccgcgtggtggccgccgacgagcgcgaccgcggcctgcggcacaccctcaactacggccacaccctcggccacgccctggagctcgccaccggtttcgcggtgcggcacggcgagggcgtcgcgatcggcacggtcttcgccggccgcctggcgggcgcgctcggccggatcggcccggaccgggtggccgagcaccacgccgtggtcgcgggctacgggctgccggtcgcgctgccgccgggcgtgccggacgacgagctgctgcggttcgtgcgccgcgacaagaagtccaccggcggcctggcgttcgtgctcgacggcccgcgcggcgcggagctggtgcgcgacgtcgacgagcacgtcgtcgccgccaccctggccgcgatgccgcgggcctga

SEQ ID NO: 52 - DNA, 1554 bp, Actinosynnema pretiosum
tgcagcccggaccgggtgcgcagggcggccgtgcaggcgcggctggcggcggccgagtggctggtgaacccgtcgacctcggccaggcaccacaccgcgctgctggcggcgttccgcgcgggcaggttgtcggtgcgcgacagcgcgacgctggtggagcacctgcgctgggccgggagcaccgccgactcggacgcggtgctcgccgggctgcgggccgacccgcgcgccgccgaggaggtgccggtgctggagcactggctggctggcgcctaccccggtgcggccaggccggggaccgcgccggggcgggacgtggactccgcgcgcaccagggcggagcaggcgccgagggcgaacgcggtgctgctggacgtgctggtggccggggacggcgaccacgtggccgaccgggcggaggcggtgctgcgggaggtgcggctggcgcgggaccccggcggctccggcggtgcggccgtgcctgcgctgtccgcgctgctgtactcggaccgcgcggacgtggccgcctcctggtgcgagcagctgctgtcggcgcgggccgtgccgctgctgccggtaccgcgcgcgcaggtgctggcgctggcggccgagtcggcgctgcgccggggcgaccacccgagggcggacgagctggcgcgggaggcgctgaccgtgctgccccccaccgcgtggggggtgtcggtggggctgccgctgagcacccggatcctggcgctgaccaggatgggccgctgcgacgaggcggcggccgtggtggcgcggccggtgccgaacggggtgttcgggcaccgcaccagcgtggactacctgcacgcgcgcgggcacttcctcctggcgcggggacggccgcgcgcggcgctgggcgacttcctgctgtgcggggagcagctgacccggtggggtctgggcggcgggtgcgcgccggtgccgtggcggaccgcggcggcggaggcgtggctggcgcagggcgacccggaccgggcgcgggcgctggcgcacgagcagctcggcaggccgggctcggacagcaggcgggcgcgcgggcaggcgctgcggctgctcgcggcgaccggcccggtgaagcggcgcccgcggctgctgcgggaggcggtggcggtgttcgaggccgtggacgacaagtacgagctggcgcggaccctgcgcgacctgggaggggcgcagcgggcgctgggcgagcacgcgctggcgcgccgggtggtccggcgcgggtggcacgtcgcccggatgtgcggggcggcgccgctgtgcgaggagctgatgcccaccgccgacgggctggtgcccgcgcagcaggcgtcggcggcctgcgggtcggacacgggccggttgaccagctcggagcaccgggtggccgcgctcgcggcgtcggggctgacgaaccgggagatcgcggtgaagctgtacgtcacgcacagcacggtggagcagcacctgacgcgggtgttccgcaagctcgggatcaagcagcgggagcagctgccgcccgcgctgagcgccgaccggtcggggtga

SEQ ID NO: 53 - protein, 5 aa, Artificial Sequence
  Synthetic
PSNSI

SEQ ID NO: 54 - DNA, 53 bp, Artificial Sequence
  Synthetic
catcgattaattaacggagaggccatatgctgcgaagcgacctgatccgtccc

SEQ ID NO: 55 - DNA, 23 bp, Artificial Sequence
  Synthetic
tgcggtaccagccgtcgcgcagc

SEQ ID NO: 56 - DNA, 82746 bp, Actinosynnema pretiosum
gatcgtgccgggtctggtggtggccctcgccgccaccggcgtcctcgcggcccccgcgagcgccgccatcaccagccccaccgtgaccagcaccgccaccaccgtcaccttcgaggtcggccgcaccgggacgcccgcgttctcgcgcgtctacgtcgacaccgaccgcagcgcggccaccggctacgcgcagggcgggatcggggcggaccacctgctggagaacggttccctctaccgccacagcggcggcggctgggcgtggacgcaggtgcggaccgtgccgttcacctccgcctcgggcgtcgcgcggtggaccgtcgagcgggccgacctcggcgagcaggcgagcccgaacgacgtcgacgtggtgttccaggtcgaggccccggtggagacctcggcgaaggtcacccacacctacaccgcgggcgcgggcggctcggtcgcctaccagccgtcgagcgcgaacgtccccaacccggaacgcggtctttaccaccacaccggcgactgcgacaaggccgacctgtccgccgacgcgctgcgccggtaccgcaccgagcagggcatcaccctcgcgatgtgcgtgttctacctggccgagcaccgctcgtccccgatcgcccagaccgccctcgaccgcctgcagcgccagttcgacgccgcgcgggtcgcgggcgtgaagctggtgctgcgcttcgcctacaccgcgtcggagtccggcgaggacgcgccgaaggagcgggtcctggcgcacatcgaccagctcgcgccgctgctgcgcgcgaacgcggacgtgaccgccgtggtgcaggccgggttcgtcggcgcgtggggcgagtggtactacaccaagaacttcggcaacgcgggcgtcgtctccgcgaccgaccaggccaaccgcaaggcggtggtggacaagctcctgaccgcgctcccggcggaccgggcggtgcagctgcgcacgccgaagttcaagcgcgccctgtacgggggcggcagcaccaggatcggccaccacaacgactgcttcctggccagcgacacggacttcggcacctacgaggacccggcgaccgaatacccgtacctggaggcggagacgcggacgctgccgatgggcggcgagacctgcgcggtgaacccgccccgcaccgactgcccgaccgcgaccgccgagctgtcccgcttccactggacgtacctgaacaccgactaccagcaggacgtgctgcgcgcgtggtcggcgcagggctgcctggcggaggtcacggccaagctcggctaccgcttcaccctgcgcgactcggtgttccccacgacgaccaccaggggcgccgcgctgcccgtgcgggtgaacctgaccaacgacggctacgccgcggcggtgaaccagcgcaccgtgaacctggtgctgcgcaacaccaccaccggcgccctgacccgcctcccgctcaccgcggacccccggacctggctgccgggcgcccgcgccatcgcgcaggacgcccagatcccgccgtcgctcgcgacggggcagtacgcgctcctgctggaactggccgaccccgccctgccgggccgcccggagtactcgatccagacggcgaacaccggcctgtgggacgcgaggacggggctgaacgacctgaagcagaccatcacggtgtcgtaaggcaggccgcggggcgcgggtaatcgctcgtgcgcggagcgcgcgggccgctagggtgcgccccatgtccgccagccggatccagtgaaccgggactcagcccagcgctagagccccgcccacactcgaatccggagagtaatcacatggttaccgcaagtgcggtcctgtgcgcccgcgcgggctgcggcgcgccccgtcccgcaggcgccacggcccgctggcgcttctgctccaccgcctgcaagcaggccgcctaccgcctgcgccgcaaagcgggcagcgcctcccacgagcacgaggacgagcagctccgcgaggcgctgctcgccctcgtggcacgggcgcggaccctgaccgaggccaccccgccctggtccgcgcaggccaccgcgctggtcgggctggcccggcgggtggtggagctggcggcggcgcgggacgccggggtcagcgggggctgagcgcgctcagcagcaccaccgcggccacagcccccgccatcaccgcggcgggggcgaacggcccgcccgtggacagggcgccagcgagggcggtcagcagggtcaccaggagcgcccgcaggcccatggtcgccgaggacgccgcgcccgccagctcggggaacaccgacatggaccggtcgaacaccaccgggtagcacacggcgaagccgacgcagaacagcgcgaccgagccggtcacggcgaccgggccgtcgccgagcagcaggaaccccagcacgccgaccacgaccgcccccaggccccaccgcaccgcccccgccgggccgccgagcagcgggatgatcccggtggcgaacgcgctggggaccgcgaacgaggcgaccacgacgagcaggtgcagcgcgaacccggtgacgccgaggccgaacgcggcggtgtagaggaacgggctggccgcgatgaacaccaggtaggcggcgaacagcaggctgggcgtcagcgacgcggccaggtaggcgcggctcccgagcagcctccggtagtcggcgagcaccgaccgggcggtgatcgcggtgcgcgcgcgggtgcgcgtctcgggcaggaacaggaacaccagcagcagggacagcgcggtggtggcgcacacggcgaggtagttgccgcgccacccggccacccggttgatcgccccgcccaggagcggcgcgcaggtcatcagcgcggacatggccgcgttcaccaagccgtacatccgcaacgccgccgggccccggtagcggtcgccgatgatcacgaacaccagcaccgcggcggcgctggccccgatgccctggacgaaccgggcggtcagcagcagcccgatcgtcggcgcgagcgcgcagcccagcgccccgaccagcatgacggcggagccgccgagcagcaccgggcggcggccgaaccggtcggagagcgggccgtggcacagcgcgcccaggcagtagccgaggaagttgagggtgacggtgagctgcacggccgggccgcccacgcccagttcgcgggcgatgtccgggaagctgggcacggacatgtcgatctcgatgatcgtgccggtgagcgcgacgatgagcagggcgggcagccacccgcccaccgcgctgtcggcctttcccactgccaacgcggtcctcccgaggtcggcgaggggttttcggcaacgcccgcgatggcgttccgtaacctttcctcgaattattcggagaaatcgccggtgcgaattctccggacctcggacgttagcgccgttcccttttccgtttaactcattttccgctctcgtcgaacgggctgcccgcctcctccggctgccccgcgagcagccggtcgaccagtgcggtgacgaggtcgcggcgctggtggtcggtcagcaggtcggggagggtgaggcgttccaggatcagccagttgagggccagccacagcagcgccacggtggtgccgtcgccgggcaggccggaggcggcgtggtgctcggcgttgaagcgcaggtcggcgcggacgcgggcggtcaggacctcgcgcagctccgggttgcgcgcggcctccaggcgcagttccagcagggcgaggaaaccggtcgggaaggccgagacgcggtcgacgagggcgtgcatcaattcggtgacgcgggaccggtcgcgcgggccgtccggggtctcgcccagcggggcctggtcgagcagccgctggtagacccgctcgcccgcgccggtgaggatctcggcgcgcccggcgaagtagttcgacgcggtgcccgcgggcaccccggcccgctggtcgaccgcccggaaggtgagcccccgcgcgccctcgcccgccagcacctcgacggcggcgtcgagcagcgcggcccgccgctccgggttgcgtcgcgccattgacaccactccaaacgtagtactacggttcgaaccacttcaagcagagtactacggccggagtggtcgagacgagggggaagcccgtgcgcaagctcgtgtacttcatcgcggcgagcatcgacggcgtgatcgcgggcccggagggcgagcacgacttctacccgaccgcagagccgatggtcgagcacctgcgcgccgagtaccccgagaccacgcccacccacgtccgcccgctcgtcgggatgcccctggacaccccgaaccgacgctacgacaccgtgctcatgggccggggcagctaccagcccgcgctcgacgcgggcgtgggcagcccgtacgcgcacctgcgccagtacgtggtctcccgctccctgcccgacgccgatccggccgtcgcgctggtgcgcgcggacccggtcggcctggtgcgggagctcaagcgggaggacggcctggacgtgtggctgtgcggcggcgcggacctggccgggcagctgctcccggagatcgacgagctggtggtcaagacctacccggtggtcgcgggcggcgggaccccgctgttccgcgccggtttcgccccgcgcgcgttcaggccggtcgacgtgcgcgcgttcgaccacggcggcgtggtcaccacctaccgcccggcctgacgggcgggccaccgccccctgcgggcgtgcagcgcaccggccggcagcgcggcggtggtggacgcgagcaggacggcggccttggcctcggccgcgccaccggggccgcgtacgacgccgtcgcccaccgctacgcggacctgttcagcgacgtcctcgggtccctccccctcgaacgcgccctgctgtccgcgttcgccgagctcctgggccccggcgcccgcgtggcggacctcggctgcggcctcggccacctcaccgcgcacctgcgcgcgttcgacctgcccgacgaccgcctcgccggaatcctggcgttctgctcggtgatccacaccccggcccgccgaactcccgcccgtgctcgccgagttccaccgggtgctcgaacccggcggtcaccccgtgctcggtttcttcgcctgcgacgatccggaaccgcgggccttcgaccacaaagtcgtgaccgcctaccgctggtcacccgccgccctgctggcgctgctgcaagagcgcggttccaccgaagtggcccggctggtgcgggaaccgcaggtcggcggttgtcaccacccccgaccgcgacattccactcccctgggcacgcccccgcggtgggtacagtggaactcctgcgtggtgcgcgccgaatcggcgcgggcagttcccaggaggagcggcacacgatcatgggttgtggattggctattccgcggacggccgtcggcgccgcccggtccggtggtggggcgtgaccgaggccttcacgtgggagaccgacgaggacatcaagctggcggccacgctgcgccgcgacgcggaggcggcgaacgcggccgactccacccgcgcctacctcaaggcgatcggccgggtgcggctgctgaccgccgaccaggaggtggagctggccaggcgggtggaggcgggcctgtacgccgagcacctgctggacctgccgcccgcggcgcgcgggtcgcggcggccggtcgagctgcggcgggtggtgcgggacgggcagcgcgccaaggaccacctgctggaggcgaacctgcggctggtggtgagcatcgccaagcggtacaccggcaagggcatggcgctgctcgacgtcatccaggagggcaacctgggcctggtgcgcgcggtcgagaagttcgactacgccaaggggttcaagttctccacctacgccacgtggtggatccggcaggcgatcacgcgcgcgctggccgaccagtcgcgcaccatccggctgcccgtgcacatggtggaggtggtcaccaagctggcgcggatcgagcgggacctgctgcaccgcaacgggcgcgaggcgaccgccgaggaggtcgcggtcgagctggacgtgacgccggagaaggtcgtggagctgcggcagcacgcgcgcacgccggtgtcgctggaccagaccatcggcgcggaggcggacacctccatcggcgacctgatcgcggacgagaacgcggtggccccgctggaggcggtcagctcggcgctgctgtcggcggacctgcgggcggtgctggagacgctgacgccgcgcgaggcgggcgtggtgcggatgcggtacggcttcccggacggggtgctgcacacgctggacgaggttgggcgggtgtacggggtgacgcgggagcgcatccggcagatcgagtcgcgcaccatggcgaagttgcagcacccggcgcgctcgcgcgggctggagggctacctggactgagggggcgcggggcacgcgccccctcggtcgagggcccgctcagaacacgtccgtcgcgtaccgccccccggcgcggagcgcgtccagccacccggcgccgggaccgatgcgccccagcacctcctccaccgcgggtcccacccgcgtcgcgtcgccgcagaggtacacgcgcgccccggcgtccaccagctcgcgcacccggtcccgccgctgccacagccggtgctgcacgtggcggacctcgccatcggggtggcgcgagtaggcccggtccacctccagccagtcgccgagcgcggcgaactcctccgcgtacaggtcgtccagctcgggcccccggcagccgaacagcagcagcgccgggcccggttcgccgccgagcgcggcccgcgcccgcaggaacccccggaacggcgcgatcccggtgcccgcggcgatcagcaccacggggacggtgttgtcggcgggcgggcggaacgactccggcggtgacgcgatcccgacggccacgcggtcgccgggtcgcacccggctcaggtagccggaggtcacgccggtgaccgacacggtgagcgccacctcggcccgctgctcctcggccgccgagctgatcgagtacaggcgggtcctgggcgcgggcagcagttccagggccagcgccaggtcgaccgcgcacgaggggaagcgctccagcagctccagcagcgtcgccccggtcgtggcggccagctcggcgcgctccggcgggcaggggcagcgctcggccaggcgcgcgacgaccccggcgcccgcgggcgcggacaggtcgacgcaccgggtcagcagctcgtccacgcgcagcgcgcgcccgaccggcaccagcccggtgggcgcgctcgactcgaccgtcacgacccgctccgcgcgcgtgcccagccgcgccaccgcccgccgcaccaggtcggggtggttctgcggcagcacggacaggtagtcgccggtgcggtaggacgtgccgtcgggcagccgcagctccaggtgccgcttggacccggcgtccggtccgcgcaccagctcccggttctccagcacgaccgccgacgcgagatcgccgaggccgtcggccgcgtccgactcgaccacccggaagcgcggcccgatctcgcccacctcgaccccgcactcggccgacagcagcggccacagcggctcgtaccagcgctcccagtcgccgaagaagtccgcgcgggcgtcggtggccccgcgctcgcgcagccgccgggcgcccgccgccgccatggcctcgtcgatcagggtgggcacgcgctggtaggtggccgaccagtccaggttgccgcagccgagcaccaggtagtccactccggacaggtcgggcccgtcctgcgtgagccaccgcacgaagtgggccgcgttgtccggcggggcgccgttgtaggaggagctgacgatcaccaccggtcccgacgcgggcagggcgcgcaccgcgtcgtcaaggggcgcgacggaagtcgcccacccgcgagccgcaccgtcaccggcgatcgtgcgcgccaggccctcgccgctgccgccgttcgacccgtagaacacgtgcagcggggttccgtgccgatgggtggccggaacctggtgcggcacaacggtttcgacggtcgcggtggcgccagggcggtcggcgcgcggccgggcgcggaccaccagcgagtccggcttgagggtcaacgtctgcttgatcgtcagccggtggtcggggtcggcgagcgcgaggtcgaaccgctggagcaccagcgccagcacgagggtcgcctcctgcagggcgaacggccgcccgatgcacgcccgcgccccgtgcccgaacggcatccacgcgcaggccgggatctcgtccatccgctcgggcgcgaaccgctccgggtcgaacacgtcggggtcgcgccacaccgccgggtcccggtgcagcgtcggcagcatcaccagcaccggctcgcccgcgccgatcgcgtggccgcccagctctgcgggctcgtcgggggccagggcgaacgcgggcgcggtcgggtgcagccgcagggtctcgcgcagcacctgcccgaggtagtcgagccgggccaggtcctcgaacgcgggcacgcggtcgcccaggaccccgtccaccgcgtccttggccttgcgcagcacctcggggtgcgccagcagccggtgcacggcgaaggagagcaggccggaggtggtctcgtgcccggcgatcaggaacgtggccagctggtagcgcacgttccgcgcggacagccgctccccggtgaccgggtcggcgcaggtgagcatccgctccagcaggtcgtccggccgctcccctgcgggcagtgcggcgcgcgcggcgacgatcccgtcggcgatccggttcatggtggcgatgtcgtcgcggtaccgctggttcctgcccaccaggaacggctgcacgcccggcagccgctcggcgcgctcgcccgcctccacgaggctgcggaccatcgcgtcgacgaaggggtgggcccggtcgcggtagaaggagttgaaccgggcgccgaaggcgcacagcgcgatggtgtccagggtcagccgggtcatgtcgtcggcgacgtcgatgcggtggtcggggccgaaccgccgccagcgcacgagcatctgctcggcgatgtcgagcatggccgggaagtggtccctcatggcggtggggccgaaggcgggcatgagcagccggtgcgccttgccccagttcggctcgtcgccgcgcgcggtgaacagcccgtcgcccgcgaagtcgcggacctgctccagcgcggggtgcacggccttgccccagctcgggtcggagcacatgcgggcgacgagctcgtggctggaggcgaagacctggcgctgcccggcgaaggtcagcgcgacgatgccgccgtgctcgcggtggcagcggtcgaggaagtcgacgtcggtgtccaggtcggaggtgaggacgtcgagcaggttccccaccaggggcaggggcttggggccggggatgcgcgtgccggtggcgaccacggggtcccccttcggtcgacgggtgccgggtcgcccccacggtaaagttggccaagtgaccaagtcaagcgacctggtcagttggccagactgttaggatgaccccatgtcgacggcacaggacgccagggcgggccggaagcacgtgagcgcggccgtgcggcgcgagcagctcgtggccgccgcgctgcgcgtgatgaagcgcgacgggatcgccgcggcgaccacgcgcgcgatctgcgccgaggccgacatgccgcacggcgcgttccactactgcttccgctccaagcaggagctgtacaccgcgctgctcaccacgggcctgaacaccagcctggacgcggcgtggcccgcgatctcgccggacctgcccgccgtggacaacgtccgcacgatgctggccgccttctggaccgcgttccaggccgacccggaggcggagctggtgctgggcgagctggtcaccctcgcccagcgggagcccgagctgcgcgagctgcccgcgtggcagcaccgcgagtacgtcgccaaggcggtcgcgcacctggaccggctcgccgaggaggccgggttcgactactcggtggaccgggcgctgctggcggagctggtggtgtcggtgctggacggggtcgcctcgtcctggttctcgcaccgggacggggagctggcgaaggcgtcgctggaccagttcgccgtgatgttcgcggccctgacgcgctgagccccgcgcgcacccgcgcccggccggccgctgacccgttcgggccactgcgccgaccagatcgtttacccccctgcccgcacgggtagccgcgcacaccgggcagccgcccggacgtccggacacacctgcgccctggaggcaggcatgaccgacacgacgacgcgccaccgcacgccggtgcagaacgccgctttagcggtgggcgcggtgttcctgctggtgggcatcgccgggttcgtccccggcctgaccacgaactacgacggcctggagttcgcgggccaccactcggacgccgccctgctgggcgtgttcaaggtctccgcgctgcacaacctggtgcacctcgcgttcggcgcggcgggcatcgccgcggggctgtccaaggccccgcgcggggcccgcgcgttcctgatcggcggcggcgcgatctacgcggtgctgtggctgtacggcgtggtcgtggacgagcacagcacggccaacttcgtgccgctcaacgccgccgacaactggctgcacctcggcctggccgtcggcatgatcggcctcggcgcggcgctgggccgcaacgcggcgcactaccgcgcggcgcggtccgacgactgacccgccaccggcagcgggggccgaccggaaagcccggtcggcccccgtccgcggtcaggaccgggccgcgcgcagcccgccccgcaccgccccgcccacccggtgcgccccgctcaccagcgaggtcgccagctcccccggcctgggcacgcgggccagcagcgccgtcaccgggctggagtccagcacccggccggggtaggacccgtgcacccgcccgtcctcgtccaccggcgagtccacgttgtccaccccgttgtccggcagctcggaccgcatccccgaccggccgaccgcgcgcaccgacatcagcgcgtcggtgagcgccgggaacagccgctgccccaggtagaacgcgagcgcagcgtcgcccaccggcacctcgcggcgggggcgctcggcggcgcgcagcacggcctcggcgaccagctccggcgcgtacaccgggggcggcggcttgggcagcgagccgacgcggctgcggcagtgctcgaagaacgggctgttgatcgcggcgggaagcacggcggtgaccgcgatcgactcgccgtcgtgggccagctccacccgcagcgcgtcgtagaacgcccgcaccgccgccttgctggccgcgtacggggcctggagcggagccgagcgcaggccgagcacggacgcgaccccgatcagcactcccccgcccgcccggcgcagcgcgggcagcgcggccttggccccgtgcacgtgcccgaggtagttgacccgcatcacccggtcgaactccccggcgggcgtgtcctccacccgcccgtacaccccgatcccggccgcgttgacccaggtgtccacccgcccgaaccgctccaccgccacgtcggcgaccgcgcgcacggccgcctcgtcggcgatgtccgccgtcaccgcgaccgcccgcccacccgcgccccggacctcctcgaccagcccgtccagggcgcgggtgttgcgcgccgcgcacaccacgcgcgcccccgccgccgcgaaccgcagcgcggtgacccgcccgatccccgacgacgcgcccaccacgacgacgacctgctccgccaacctcctgggcatgtggcccctcctcgtcccggatcgcgaaatgcggcgggtacccggaggaacgccgtccacgcgtcccggaggggtgtccggcgaggaattgcgccatcgggttcccccgcgcatagccgcccggttcacgggtagttcccaccgcgacaggcaggaccgaccagcggaggaggagacccatggccaccggtgagaccggcttcgacgacgtcagcttcgacctgatctcggtgcagtaccactccctcaaggcggggcacgactacggccagtacgtccgcgacgccgacaacgcgggccgccaggacatcgcggacttcttccgccgcgtcatggacgaggacgccgcgcgcgccaaggagtgccaccggttcctggccgagctgaccggcagcagcgagtccggcccggcgatgagctgaccgtccgcaaaggactgtccactgcgggcaccagcgcggtccccggccggtgggtcggccggggaccgcgctcgcccgcccgctgacccgcgcgatcacaccaccgcgacgctccgcgcggcgtcggcgatctgctccaggcgggtcaccacctcgcccaccgggggcagcgcgagcagggtccggcgcgcgtggcgcgccctcgcggtgaagcggggatcggccagcacggtccggcaggcgtccgccagatcggcgggcgagccgtccaccacccgcacgcccagccccagcgcggcgaccctggcggcgttgtgcggctggtcgccgaacagcggggcggcgaccatgggcacccccgcccgcaccgcctcgcgcacgctgttggccccggcgtgggtgacgaacagctgcgcgcactccagcagcagcggctggggcgcccgctcgaccacgcgcacgtgcggcgcgcccggcgggacgcggtcgaccgggatgcccccggtggacaccaccgcggcgcagtccacctcggacagggccgccacgagtccgcccagcgcctccaccgggtcggcgaccagcgcggtcacgtcgtcgcggcgccacagggcggccagcgcgatgccgaccgccccgtacaccagcgggcgggacgggtccaggtcggccacccacgcgggcagcgaccggccgtcgggcgacggcggccggtaggccaggcacgggctgagcgggacgccgagcagcgagtactcgcgcggcaggcagtcgacgtggccgtgccggtgcgcgccgagcgggtcggccgaggtgggcaggccctgctcggcgcggcgctcgttgagccggggcagcacggcggccgggtccagctggttcgtcgtgcccgagggcacggtcaggcagggcagcccgcgctcctccgcgacgagcaggccggccagctccatgccgtcgcgcaggaccaggtcgggccggaagcggtcgacgacggggcgcagcccggtcagcgcctcccgcagcagcggcctgcccgcgatgacgtcgacgatcgcggcgacgccggggtagtccgtgcccggcctggccggtggcgcgacgcccccgagccgctccacccagaacctcgcctggtcgggcaacgcctcgaccgagggcaggccgctgccccgcaggccgggcgcggcgctgggggccgtggccacgagcacctcgtgcccggcgtccaggaccgcgcgcccgagcacgagcatctcgtgcgcgtgcgacggggccccggtgacggtgaacagaacgcgcaactgtcttcctccgtgctggggcccgccgcgcggcgggccccggtggtgaacgggccgcggatgcggccccgaaccggtcaggcccgctcggcgcggcccagccgggtgtcgatgaactcgaacagctccgccacgcccgcgtcccggagcgcggcagcgccgtcctggccagcgccgtcccggccgtcgccctcgtcggacagggcggcgagcagggcgcgcagcctggcggcggcggacgcgcgcagcgcggggtcggcgggcgggtcggcgagcgcggcctccaggcggtccaggcccgaggtcaccgggtcgccgccgtcgagcagccccgccagcacggcggcgacctcggccgccgtcgggtggtcgaacagcagggtcgcgggcagggtcaggccggtggcggcggccaggcggtcgcgcagctccaccgccgtcatcgagtcgaaccccaggtcccggaacgcggcccgcccgtccaccgggccgtccgcgcggcccagcacggcggcggcgtggtcgcgcaccagcgccagcagcacccggtcccggtccgggccggtcaggtcgaccgcgtccggctcgggctgggcggtcctcggctcgggcagcaccacctcgggcgtccgcgcgaacacgaccacccccagctcggggctcagcggcggcgtgcccggtcccgcgcacagcagggtcgcgggcaggccgtccccgcggcggcggcgcacgagcgcggccagcaccccggcggccaccgcgtcctcggggcgcgccgcgtcgccgagcgcgcccgccggtccgcacagcgcgaacagcgcgggcggccggtcgcgggtcagctcgtccagcagcgcggccagcgccaggcccgcgtcgtccggcgcgacggcgagcacgacccagtccgggcggtggtcgtccagcgcggccaccagctccgcccggcccgagtcggcgggcagcacgaccggttcgcgcaccccgcaggccgccaccacgccccgcgcggcctccccgaccaccagcaccggccccgagggggcggcgccggtcgcggtgacgcgccgcagcctgggcgcgagcacctcgcccgcgcgcaccaccagctcgggctcgccgcagccgtgcagcgcgggcagcagcgccaccgacgccgggtcgtcgtccacgtccgccagcacgacgtccgcgcggtccggcagcgagcgcaccagcccggccaccgcgccgtccccggtgaccaccagcagcgggccgctgtgctcggccagcacgtcggcgacgctcgccagctcctcggccgaggcggtcggccccacctccagcacggtcgccgacccgccgccccgaggcgcgggcaaccgctccaggcggacctcgcaccacgcgccgggcgcctgcgccgcgggccagaacctggtgcgctggaacgggtaggtgggcaggtcgacgaccgcccccggcccgcccagcagcgcggtccagtcgaccggggccccggacgcgaacaggtcgccgatcccggagagcagcccgaccacctcgtccccgtcgaccagcccgcgcgccaccgcggcccccggcgcgacctgctcgcgcaccagggcggccagcgcgccgccgggccccacctccagcacccgcgtcacgcccagctcgcccagccgggcgacgccgtcgccgaaccggacggcctccctggcctgccgagcccagtagtccgggtcggcgagcagctcgacgtcggcgggcgcgccggtgagcgtggacagcagcgggacgcgcgggggccggacctcgacccgccgcgcggccacggccagctcaccgagcaccccgtccaggtgcgcggagtggaaggcgtggctcacccgcagcgccctggtccgccacccgcgcccggcgcactcggcggcggcccgctcgaccgcgtccccgtcaccggacagcacgaccgcgcccggcccgttgacggcggccagcgccaagcccccgcccagcagcggcgcgacctgctcctcggtggcggacaccgcgagcatcgccccgccgcgccgggccccgtgcatggcccgcgccctggccgcgacgagcgcgcaggcgtccgcctccgacagcgcgcccgcgacgtgcgcggcggccagctcgccgaccgagtgcccgagcacgacgtcgggcacgacgccccacccctccagcagccgcgccagcgcgacctccagggcgaacagcgcgggctgggcgtggacggtctccgccagcagctcggcgggcccgtcgagcacgacgtcccggacggagcgcccggtgagctcgcccagccgctcgtccagcgccgcgcacacccggtcgaacacctcggcgaacgcggggaacgcggcgtgcaggccgcgccccatgccgacgaactggctgccctgcccggagaaggcgaacgccagcccgccgggcgcgcgcaccccggtgcgggcggcggcgtgcggacgtcccccggccagcgcgcgcagcccggcgcgcagggcgtcccggtcggcggcgacgaggacggcgcggtgctcgtgcgcggcgcgcccccgcgcgagcgtggaggcgacgtcccggagcggcaggtcgtcgaggacgtcggcgagccgcgcggcctgggcgcgcagggccgccgggctccgcgcggacagcaccagcggcagcacgccgggaaagtccccaacgcgcccggctgcgttcccaccggggttttcggctgcggtccgctcggcgggggcctgctccagcacgacgtgcgcgttggtgccgctgaccccgaacgaggagaccgccgcgcggcgcggccggtccacctcgggccacgcgcgctgctcggccagcagcgcgacaccgtcccactcgacctcgggggtgggcgcgtccacgtgcagggtgcgcggcagcaccccgtgccgcagcgcctgcacggtcttcaccacaccgccgatcccggcggcgccctgcatgtgcccgaggttggacttgagggaccccagccacagcggctcgccgtcccggtcccgcccgtaggtggcgaccagcgcgcgggcctcgatcgggtcgcccaaccgggtcccggtcccgtgcgcctccaccgcgtccacgtccgaagtggacagaccggcgtccgcgagcgccgcccggatcacccgctgctgcgccgcaccgctcggcgccgtcaacccgttcgacgccccgtccgagttcaccgcgctgccccgcaccacggccagcacccgacgcccgttgcgccgcgcgtccgacagccgctccagcagcaccaaccccgcaccctcggaccacgccgtcccgtccgccgacgccgagaacggcttgcagcggccgtccggggcgagcccgcgctgcctggcgaactccacgaaccccctgggcgtggccatgaccgtcgcgccgcccgccagcgccatcgagcactcgccgccgcgcagggcccgcaccgcgaggtggatcgccaccagcgaggccgagcaggcggtgtccaccgacagcgcggggccctccagcccgagcgcgtaggcgatgcgccccgacacgacgctgcccgacacgccggtgatccggtagccgtcgtcgccgcgcgggagcacctggtcgtagtcgtggttggtgacgcccgcgaacacgccgacgtccgtcccccgcaccgagtccggggcgatgcccgcgcgctcgaacgcctcccacgcgacttcgagcagcagccgctgctgcgggtccatcgcgagggcctcgcgcggggagatgccgaagaagtccgcgtcgaacccggccgggtcgtccaggaacgcggcccggtcgacgtaggtggagccctcgtggtcggggtcggggtgcaccagccgctccaggtcccagccccggtcggcggggaacgggccgaccgcgtcgacgccgcccgcgaccaggtcccacaggtcctcgggcgaggcgaccccgccgggcgcgcggcaggccatgccgacgatcgcgatcggctcgccgggggcggacgcgggcgcgggggcggacgcgccgcccgccggtcccgaggtcccgcccacccgctccagcagcgcggcggccagctcgaccggggtcgggtggtcgaacaccagcgtcgcgggcagccgcagcccggtggcctcggcgagccggttgcgcagctccaccccggtcagcgagtcgaccccgaggtcgcggaacgcccgccgctcgtcgacgcccgcgacgggcagggcgagcacggcggcggcctcggcccgcaccagcgcgagcagcgcggccgggccccctggtaccggcgcggcgggcgcgggcgcccggcgcgacgcgaccacctcgcccagcgcggcgggcgcccggtcggcggcgcgggccagcgccagcgcggtgacggcgggccgcccgcaggccagcgcggcgtcgagcaggtcgagcgcggcgggcgcgggcagcgggcgcatcccggacgcggccagccgctcccggtcggccgcgccgcgctcgcccaccagcgcgctgtccacgtcccacagcccccaggcgatggacgtggcgggcaggccgagcgcgtgccgctcgtgcgcgaggccgtccaggaacccgttggccgccgcgtagttggcctgtccggccgagccgagcaccgaggcgacggaggagaacagcacgaacgcggccaggtcctggtccctggtcagctcgtccaggtgccgggcggccagcgccttgggcgcgaacgcggcggccaggcgctcgggggtcagcgcggtcacgacgccgtcgtccagcgcgcccgccaggtgcacgacggcggtgagggcgcgcccggcgagcgcggtcgacagcgcgccccggtcggcgacgtcgcaggcgagggcctccacctcggcgcccaggccggtcagctcggcgaccaggtcgcgcgcccgctccgggccgccgcgcccgagcagcaggagcctgcgggcgccgtgctcggtgaccaggtggcgcgcgacgagcctgccgagcgcgccggtcccgccggtgagcaccaccgtcttcgccgggtccagcccggcgggcccggcgaccaccgggaccggcgtcaaccggggcacgagcacggccccgccgcggatcgcgacctgcggctcaccggaggccagcacccggtccagcaccgcgcccgcccgctcgggcgcggtgtccaggtcggccaggacgatccggccgggctcctcgagctgggcagagcgcaccagcccccacaccgccccgccgaccgccgtgccgatcggcgcgccggggtcgccggtgagcacgagcagccgcccgtccccggcgagcgtctcgcgcagggcggcgagcacccgctcgaccgagtcccccaccacggtcgtcacgtcggcgggcggtggggcctcgggcagctccaccgggcgccacgcggtctccagcagcacccccggtggaccggaccggatcccgcccgcccgtcggaaggccagctcgccgatccgcgccaccgggcggccggcgccgtcggcgaggtcgagcgagacgaccccgtccccgacccgcgccgcccgcacccgcaccgcgtccacgccggagcggtgcagcgcgacgcgggagaaggagaacggcagccccaccgcgccgccgtcccccgcgacccgctgcccggtgaagctgctcacgtgcagcgcgcactccagcagcgccgggtgcagcgcgaacccggcggccctgcgctgctcgccggtcagctcgacctcgccgaacacctcgtcgccgcgccgccacagccgggtcagccgctggaagtgcggcccgtactccaggcccttggcggcgaagtccgggtagagcccggcgaggtcgaccggttcggcgcccgcgggcggccactggcccccgaccggctcgggcgcgcggggcgggccgaggacgccgcgcgcgtgcagcaccgggggctggtcgccggagagcacgtccacggggcgcgtcccgtccgcgccgcgcgggccgacgaccacgcgcaccgccacgtcaccgctctccgggaccaccagcggcgcggccaccaccagctcctccagcgccgggcagccgacctcgtcgcccgcgcgcaccgccagctccaccagcgcgacaccgggcacgacgaccgcgccgagcacccggtggtcggcgagccacggctgggccgacagcgagagccgcccggtgaggacggcgccgtccgcgagcggcacgagcgcgcccagcagcgggtgccccgagggcacgagcccggcggcgcgcgcgtcgccggacgggcggggcgcgtccagccagtaccgctcgcgctggaagaccgtggtcggcacctcggccgtgcccgcgcccgcgtgccgggcgaggtcgacgggcacgccgcgcacgtgcagctcacccgcggacagcaggaaccggtcgagcccgccctcgtcgcggcggagcgtgcccacgacggcggtgcggtgcggtgcggcgaggtcgacgacgctggtggtgagcaccgggtgcgggctcacctccacgaacacccggtgcccccggtcgagcagggcggtgacggcgtcggcgaaccggacgggctcgcgcaggttgcggtaccagtgctcggcgtcgagccgggtggcgggccgaccggtgacggtggacagcagcgggatctcgggttcgcgggcggcgacgtcgccgagtgcggccagcagctccgcgcgcagggcgtcgacctgcggggagtgcccggcgaagtcgacgccgggcagccgccaccgcagcacccgccgggcggacaggacccgctcgaactcgcgcaccgccgcgtcctcgccggagacggtgaccgacgcgggcccgttgacggccgccacgcacacccgcccctcgaacccggcgagcagcgcggtgacccgctcgacgtcggcgacgacggtgagcatcgcgccgcggccggacagctcgcgggcgatcagcgcggagcggagcacgacgatccgggcggcctcggcgagcgtgagcgcgcccgcgacgtgggcggcggccacctcgccctgcgagtgcccgacgaccgcgtcgggccgcacgccgaccgacgcccagagcgccgcgagcgacacgacgaccgcgaacgagaccggttgcagcacgtcgacgcgctccagcgcgaccgggtcgacgagcacctcccgcagctcccacccggcgaggtcgcgcagcacgtcggcgcagcgggccatgctgtcggcgaaggcgggcgcctcccgccacaaccgctcggccacgccggtccagtgcgcgccctgaccggggaacaccaggaccacccgcccggtgacgtcggccgcgccgctcacgagccggcgcccggcctcaccacccgcgagggcgtcgagcccgccgagcagcgcctcccggtcgcgggcgaccacgacggcgcgccgctcgcgctgggcgcgggagagcagcagcggccgggcgacgtcctcggggccgagcgcgggccgctcccgcaggtgcccggccaggcgcgccgcctgggcgcgcagaccggcggtggtggccccggacacgacgaccgggacgggggcgggcacgggcacaggcacgggggcatcggacgcggcagcgggcagcggcgcagagggcacggcagcggtcgcggcgaaggttccgggcgcggcggtcacgcccggtgcgccaaccgcgccgggcgcggcaggtgagccggtcgtggcgggtgccacggcctccgccacggcctccgccacgggcgcctcttcgaggatcaggtgggcgttggtgccgctggccccgaaggccgacaccgccccccgcctcagccgcaccgccgcggcgcccccaccgactccacccgccacgcccccggcagcaccacccggtcggccccccgcagcaccaccctccaagccaccagcagcatcccccagtgcactccctggctcccctcccccacgccaaggcagggcccggtcgagcacccgcaccgccccggcgtcccagtcgacctcggtggtcggcgtcgcggcgtgcagggtggggggcaggacgccgttgcgcagcgcctgcaccaccttcagcaccccggccacgcccgccgcgccctgcgtgtgcccgatgttcgacttcaccgacccgagccacagcggcccggcgcccgcgcgaccgcgcccgtaggtctccagcagcgcctgcgcctcgatcgggtccccgagcacggtcccggtgccgtgcgcctccagcaggtccacgtccgccgccgacagcccggcgtcggccagcgccgcccggatcacccgctgctgcgcgggaccgctgggcgcggtgagcccgttggacgcgccgtcggagttcaccgccgacccgcgcaccaccgccaacacccgccgcccgtcccgccgggcggcggagagcggttgcagcagcaccagacccgcgccctcgccccacgcggtgccgtccgcgtcggcggagaacgccttgcagcgcccgtcgcgggccagcgcgcgctgccgcgagaactcgacgaacgtgtccggcgtcgacatcaccgtcaccccgcccgccagcgccatcgagcactcgccgcgctggagcgcccgcaccgcgaggtggatcgcgaccagcgacgacgagcaggcggtgtccaccgtgatcgcgggcccctccagcccgagggtgtaggcgacgcgcccgatcgcggcgctgccggagctgccggtccccaggtagccctccagctccggcggcacgggccggaaccgcgagccgtggtcgtggctgagcacgccgccgaagacgccgacgtcgcgcccgcgcaccgagtccggggcgatgcccgcgcgctcgaacgcctcccacgcgacctccagcagcagccggtgctgcgggtcgatcaccagggcctcgcgcggcgagatcccgaagaaccccggctcgaaccaggccatgtcgcgcaggaacgccccgtgccgcacgtaggagcgcccgacccggtcggggtcggggtcgaacaggtccgccaggtcccagccccggtcgcctgggaacgggcccgccgcgtcgacctcgccgacgaccaggtcccacagctcctcgggcgaggtgaccccgccggggaagcggcaggccgcgccgacgatcgcgaccggctcggccgcgcccccggtggcgcgcagccgctcgttctcccgcagcgaggcgcgcagcgcgtccaggagctgctcacgggtggtcacgcgggtcctccaaagggggttcggctactcgccgccgagtgcgcggcggaccagctcgtcggcggccatgtcgtcgaagtcgtcgccgtgccggggccccggcagccggtcggccgggaacagctcggcctccaggcgctcggcggtggcggccggggtggggtggtcgaacaccacgctcgcgggcagccgcaccccggccagccggacgagccggttgcgcagcgccatcgcggtgatcgagtcgaacccggcgtcgcggaacgcctgcccggcgggcagcgcggccccgtcgtcgtggcccagcacgcccagcgcctcggcggtgaccagctccagcaggtgggcgcgccgctcggcgggcggcatcgggcgcagccggtcggccagcgacggcccggccggggcgggggtcgcggcgcgcggggcggggacccggcgctccggttcgcggtcgagcgggacgagcgcgggcagaccggtgcgcagggccgcgtcgaacaggccgagcgcgacctgcggcgagagcgttccgcgaccgcgcgccatgccctcggtccacggcccccaggccagcgccgtggcgggcaggccgagggcgtgccgctcgtgcgcgaggccgtccaggtgggcgttggccgccgcgtagttggcctgcccggcgtggccgagcgcgcccgcgaccgaggagaacagcacgaacgccgccaggtcccggtccctggtcagctcgtccaggtgccgggcgccctcggccttggggcgcagcaccgcgtccagccgctcgggggtcagcgcggtcagggcgccgtcgtcgagcaccccggccaggtgcgccaccagcgtcagcggccggtccggcggcacggcggccagcgccgcgaccacgtcggcccggttcgcgacgtcgccgcgcacgtgccgcacgtcggcgcccggcacgtccggtccgcgcccgctgcggctgagcagcaccaggcagcgcacgccgtgcgcgcgcaccaggtgctcggccagcagcgcggccagcgcgccggaaccgccggtgatcagcgcggcgccgccggggtcgagcacggccccgcctgcgccgggcgcggagacccggtgcagccccggcacgagcacgcgtcccgcgcgcagcaccgcctccggcagcgcgccggtggcgagggcgggcagcagcgcggcggactcgggccggtcgtccaggtcggccaggacgaaccggcctgggtgctcggcggcggccgaccgcaccaggccccacaccgcggcgccctcggcgcgtccggcgcgcggccccaccgcgcccgaggtcgtcacgagcagccgcgtcgactcccagcggggctcggtcaccacctcctggagcagcggcagcaggtcgcggacgtgcccggcgtcgacccgcaccaccggcggcacccgctcgccgacctcgcgcagcggcacgggctcggcggcgccgggcggcgggccgacctccgtccacgcctggcgcagcagcacccccggccgcagccggtcgggggtgaccggccgggacaccagcgaggcgatctccagcagcgggcggccccgcgggcaggccacgcgcacccggacgccgccgtcgccgtcgggccgcagggaggcgcgcaccgcgcggcccgcccggcccgcgccgcgcgcggagcgccagctgaacggcacccgcacctgcgagccgtcctcgacgccggtgagcgcgatggcctggagcaccgcgtcgagcagcgcggggtgcacggcgtgcccgtcgtcggcgacctcgtccggcaggaccgcctccaccagcacctcgtcgtcgagcttccaggcggcgcgcagccccaggaacgcgggcccgtacccgtggccggtggcggccatcgcggcccggtggtcggccaccggcagcggcagcgcgcccggcggcggccactgcgccaggtcgaacgcgagcggccccgactcgggggcgagctggccggtgacgtgccgggtccacggtgagcgcggccctgccgactccggccgggagtgcacggccacctcccgccgcccgtccggtcccggcgcggcggcggtgacgcgcagcagcgccgccccgtcgccgagcaccagcgggacctcgttgaccagctcggcgacctcgggcagcccgagctggcgaccggccgcccgcaccaggtccagcagggccgtgccgggcacgacgaccgcgtcgagcagccggtggtcggccagccacgggtgggcgccggtggcggcgcggccggtgaacgcggcggtgtccgagccggggatctccagctccccggtgagcagcgggtgctccagcggggcggcgtcggtggtgggcggggcggggtcgagccagaagcggcggcgctggaacgggtaggtgggcaggtcggtggtccggccgcccgcgtcggccagcagcggggcgaagtcgacgcgcgcgcccctcgcgtgcagcgcgccgagcgcggtgatcagggagtggtcctcgtcccggtcgcggtgcagcaggggggcgcagagcacgtcgtgcccgtccaggcactcctgcgcggcggcggccagcccaccaccggggccgacctcgacgaacgcggtcgcgccccgccggtgcaggtcggtgacggcgtcggcgaagcgcaccggctcgcgggcctggcccagccagtactcgggcgcgtcgagcgcgccgggcgcggtggtggccaccgggacgcgcggcgggcggaagtccaggcgcgccagcacgtcggcgaacgcgggcagcaccgcgtcgacggcggccgagtggaaggcgtgggcgacccgcaggcgcgtggcgcgcccgaggcgggcggcgacctcggtgacggcggcctcgtcgccggtgagcacggtggcgcgggggccgttgacggcggccagctccaccccgtcgaccagcgcggcgcgcgcctcgtcctcgcccgcgcggaccgcgaccatcacgccggggggcagctcgcgcatgagggcgccgcgcgcggccacgagggtggaggcgtcctccagggacagcaccccggcggcgtgcgcgacggcgatctcgccgaccgagtgcccggcgagcaggtcggggcggacgccgagcgcgtcgagcaggcgcaccagcgcgacctgcacggcgaacaggccggcctgcgcgtagcgggtgtcggcgagcagcccggggtcgtccagcgcggcgcgcacgccgggaccgccgacgacgcgccggtccagcagggcgcacacctcgcgcagcgcgtcccggaacaccgggaaccgccgctccagcagccgcccggtcccggcccgctgactgccctggccggacagcaggaacgccacgcccccgccgtcccgctcggcgggcgcggtggcgccggacgcgaggtcgcgcagcgccgccagcgcctgaccccggtcgcgggcggggatcaccgcccggcagggcagggcggcgcggccggtgaccagggacagcgccagatcgggcagcgggacgcgctcggcgaccggcagcaggcgggccgcggtggcgcgcagcgcggccgggtcgtgcgcggacagcacgagcggcaggtgaccggggacgtcccggtcgggcccggcgggcacccggtggtcgggcgcccggctctcggacacctggcccaggggcgcctggccctcaggcgcccgccccatggacgcctggccctcggatgtccggtcttcggatgtctggtcttcgggcacccggccgccgaacgcccggccctcggatgtctggccctcgggtgcccggcctccggacgtccgtccctcgggcgcccgcccctcaggcacccgcccctctgaagtccgcctgtcggggatccgcacctcggggatccgcccctcggggctcaactcctcgggcgcctgctccagcaccacgtgcgcgttggtgccgctgaccccgaacgaggacaccgccgcccggcgcggcctgcccacctcgggccagtcccgctgctcggccagcaccgcgaccccgtcccactcgacctcgggggtgggctcggccgcgtgcagggtcgcgggcagcaccccgtgccgcagcgcctgcaccgccttgatcagcccggcgaccccggcggcggcctgggtgtgcccgatgttggacttgaccgacccgagccacaccggctccccgccccggtcccgcccgtaggtcgccaccagcgcccgcgcctcgatcgggtcgcccaaccgggtcccggtcccgtgcgcctccacgaggtccacgtcggccgccgacagcccggcgtccgcgagcgcccgcccgatgacgcgctcctgcgcggggccgctgggcgcggtcagcccgttggaggccccgtcggagttcaccgcgctgccgcgcaccagccccagcacccggcgcccgttgcggcgcgcgtcccacagccgctccagcagcaccagcccgacgccctcgccgaacccggtgccgtccgcgcccgccccgaacgccttgcagcgcccgtcggcggcgagcccgcgctgccgggagaagtccacgaacaggtccggcgtgctcagcaccgtcacgccgcccgccagcgccagcgagcactcgccgccgcgcagggcccgcaccgccaggtgcacgcccaccagcgacgacgagcaggcggtgtccacggtcatcgccggtccctgcagcccgaaggcgtaggcgacccggccggacaccacgctcccggacaccccggtcacccggtgcccctccagcccctggtcggagtgggcgggcagcaccgggtagccgtgcgcgatgacgcccgcgaacacgccggtgtcggtgccgcgcagcgagtcgggggcgatgcccgcgcgctcgaacagctcccacgccacctccagcagcagccgctgctgcgggtcggtgccgatggcctcgcgggggctgatgccgaacaggtcggcgtcgaagcccgcgacgtcggcgaggaacccgccctcgcgggcgtggctgcgcccggcgcggtccgggtcgggggcgaacagggccgccaggtcccagccccggtcggcggggaacggggtgagcgcgtccccgccgcccgcgacgaggtcccacaggtcctccggggaggacacgtcgccggggaagcggcaggccatcgacacgatcgcgatgggctcccccgcgtcggcggtgacggcggcggtggcggggggcgcggtggtcccggcggcgggggcgtgcccgagctcggcgagcaggtgcgcggtgagcagcgccggggtcgggtggtcgaacaccgaggtggcggcgagcgcgagcccggtcgcggcggcgagccggttgcgcagctccacggccgtgagcgagtcgacgcccagctccttgaacgcgcggttcgcgtccacggcggaggcgtcgcggtgcccgagcacggtcgcggtgtgcgccctgaccacgtcgatcagcggccggtccgcgccggcgacggcctgggccgcgaccgcccggcgcggtccggcgaggtcgcgcagcagcggcgggacctcggcggcgcggcgggggtcgagcgcggcggcgaggacggcgggttccggcgaggcgagcgcggcgtcgagcagggccagcgcctcggtggtggtgagcgagcccgcgccgtgcctggccccgcgcgcgaggtcggcggcggtgagcccggcggtgagggcgctggtgtgctcccagtggccccaggcgatggaggtggcgggcagcccggcggagcggcggcgggcggccagcgcgtcgagcgcggcgttcgcggcggcgtagttggcctggcccgcgttgccgagcgtcccggcggcggaggagaacagcacgaacgcggccaggtcggcgtcgcgggtcagctcgtccagcagcagcgccccgtcgagcttggggcgcaggaccgcgtcgacgcgcgccgcgtcctggtcggcgagcagggcgtcgtcgagcgcgcccgccgtgtgcacgacggcggtcagcgggcgcccgcccaggtcggccagcagggcggcgacccggtcgcggtcggcggcgtcgcagcgctcgaaccgcgtggtggcgccgagttcggcgaggtcggcggccagctcggcggcgccgggcgcgtcggggccgcgccgggaggtgatcagcaggtggcgggcgccgtgcgcggacaccaggtggcgggcggtgagcgcgccgagcgcgccgccgccgtggaccaggacggtgccctcggggtcgagcgggcgggggacggtgagcacgaccttgccgacgtgcccgccggacgcgacgtgccgcagcgcctcgggggcgtgccggacgtcgcggacggtcagcggcagcggctcaaggcggcccgccgcgaacagccccagcacctcggcgagcagttcggcggtgcggtccgggcccgcgtcgcgcaggtcgtaggcccggtaggtcacgtcggcgcgcacgaggtcgcgcacgtcgaccttgcccagctccaggaaccgcccgcccggcgcgagcagccgcagcgaggcgtccaccagctcgccggtgagcgagttcagcacgacgtcgacgttcgggaagcgctcggcgaacccggtgtcgcgggaggaggccaggtgcgcgtcgtccaggccgagcgcgcgcagcaccggccacttcgccggggacgcggtggcgaacacctccgcgcccagcagcgcggccagcctgacggccgcgccgccgacgccgcccgcgcccgcgtgcaccaggacgcggtcgcccggcccgacctgggcgaggtcgcgcaggccgtagagggcggtgaggaacgcgaccggcaccgaggcggcgcgcgcgaacccccagccgggcgggacgggcgcgagcagccgggcgtcggtgaccgcgaccggcccgaacgcgccgaagtcgcggtcgaacgtgccgagcccccggtcgccgaccgcgaggccggtgacgccggggccggtctccaggaccacgcccgcggcctcgccgccgagcccgcgctggccgggcacggcgttcagcacgacggcggcgtcccggaagttgacgcccgcggcgcgcacgcccacccggacctcgccgggttccagcgggcggttcgcggccgggctcggggtcgcggccaggtcggtgaacgtgccgccgtcgcaggtcaggcgccaggtcggggcggcgggcagcggcaggccgtcgtcgcgggtgcgcgccaggcgcggcgcgctcgccctgcccgcgcgcacccggacctgcggttcgccggtggcgggcagggcgggcagcagggcgcgcgaggcggggtcgtcgtccaggtcgaccacggcgaagcggcccggccgctcggtctgggcggtgcgcagcagcccccacacggccgcggcggccgggtcctcggccgcgccgcgcgtgacgacggtcagctcgtcgtgctcgcgcagggcctccagggtcaggccgacgagggtgcgggcgtcgccgccgggcaggtcgagcacgggccggtcgagcacgggggcgcccggcgtggagtccggtgtggacaccggcaagtccaggtcgacccactcgacgcggaacagcgcgcccgagccgacgagcgcggtggccggggccgggcgcagccgcagggcgtcgacctccacgaccggcgcgccctcggggtcgtaggcgcgcagcacgtggtcgtcggcggtcccggtcaggcgcacgcgcagcgcggtcgccccggtggcgcgcagcctggtcccccgccaggagaacggcagcagcacctcgtcggcggcgacccgcgccgggggcagcaggccggtggcgtgcagcgcggcgtcgaacagggccgggtgcaggccccagccgtccgggctcagcggtgcgggcagctccacctcggcgaacacctcgccgtcgcgggtccaggcccgtcgcagcccgcgcagcgcgggcccgtagtcgtagccctcggcggcccggctgtcgtagaacccggccaggtcgacctcgcgcgcgccggtcggcggccactcggtcagcgggaccggggtggagcggtcggcggcgagggagccgctggcgtgccgggtccacgcgccgccctcggcgcgggcgtgggcctccagcggcctgcgcccggcggcgtccggggcgccgatcctgagctgcacctccaccgcgccgcgctcgggcagcaccagcggtgcctccacgagcagctcgtccagcgcgggcgcgccgacctcgtcgccggcgcggatggccagctccgcccagcacgtgccgggcaggacggcggacccgcgcaccgcgtggtcggcgagccacgggtgccgcgcggtgctcagccgcgcctggcacaccaggtcctcgccacccgcgaggtcggtggcggaggtgagcagcgggtgccgcagcgcggaggcggcggtggggtcggcgtccagccagtagcgggcgcgctggaacgggtaggtgggcaggtcgagcgcccggaccggcgggggcgccagcggggagaggtcgacgggcacgccgcgcacgtgcagctcggcggcggagcgcaggaaccggtcgaggccgccgtcgtcgcggcgcagggtgccggtggcgaccgaggggcccgcgcccgcgagcaggtcctggacgggcgcggtgagcaccgggtgggcgccgacctccacgaacgcccggaacccgtcggcgaccaggagcgcggtggcgtcggcgaagcggacggggtggcgcaggttggcgaaccagtagtcggcgtcgagctcggcgccgtccagccacttccccgtgacggtggacagcatcgggatggcggccggtcggggggtgatcggcgcgaggtcggcgagcaggccctcgcgcagcacgtccacggcgggcgagtgcgaggcgtagtccaccggcagcctgcgggcgccctccacgcgggcgaccagcgcgtcgacctcgccggagacgaccaccgaaccgggtccgttgaccgccgcgacgcacaccccggtgcccgcgaccagctcctcgacctgggcctgcggcagcgggatcgaggccatgccgccgccattcagcccggcggcgatggcccggctgcgcagcacgaccacgcgggccgcgtcggccagtgagagcgctcccgcgacgcaggcggcggcgatctcgccctgcgagtggccgaccaccgcgtcgggctcgacgccgaccgagcgccacagccgggccagcgcgaccatgacgacgaacgcgaccggctggacgacgtccacccggctcaggtcgccgccgcgcagcacgtcgaccacgcggaagtccaccagggggtcgacggccgccgcgcactcgcccacccgctcggcgaacaccggggactcgtcgagcagccgggcgcccatgcccacccactggccgccctggccggggaacacgagcaccgtgcggccggtgacgtccgcgcgcccggtgaccagggcgggcgagggctcgtcggcggccagcgcctccagcgcggcgagcgcgcccgcccggtcggcggcgaccacgacggcgcgctcgccgaggtcggcccggccggctctcagcgcgcccgccagctcggcggccgtgacgtcggagggggcgaggaagtcggccagccgccgggcctgggcgcgcagcgccgccgcgtcgacggcggacaccggcagcggcacgggcccgcgctcgggcgcggcgggcgcggcggtctcggcgggcgcctgctccaggaccacgtgcgcgttggtgccgctgaccccgaacgaggacaccgccgcgcgacgcggccggtccacctcgggccacgcgcgctgctcggccagcacctccaccccgtcccagtcgacctcgggggtgggcgcgtccacgtgcagggtgcgcggcagcaccccgtgccgcagcgcctgcacgagcttgatgagcccggcgatcccggccgcgccctggggatgcccgaagttcgacttcagcgagccgagccacagcggctcgccgtcccggtcccgcccgtaggtcgcgaccagcgcgcgggcctcgatcggatcaccgagccgcgtcccggtcccgtgcgcctccaccgcgtccacgtccgaagtggacagaccggcgtccgcgagcgccgcccggatcacccgctgctgcgccgcaccgctcggcgccgtcaacccgttcgacgccccgtccgagttcaccgcgctgccccggatcaccgccagcacccgacgcccgttgcgccgcgcgtccgacagccgctccagcagcaccaaccccgcgccctcggaccacgccgtcccgtccgccgacgccgagaacggcttgcagcggccgtccggggccagcgcgccctgccgggcgaactcgacgaagtcggcgggcgtggccatgaccgtcgcgccgcccgccagcgccatcgagcactcgccgcgccgcagcgactgcacggccaggtgcaccgccaccagcgacgacgagcaggcggtgtccacggtgagcgcgggcccggtgagcccgaacgcgtaggcgatccgcccggaggcgacgctgcccgcgttgccggtgctcaggtagccctccacgagctgccggtccagccccggccgcgacccgtagtcgtggcccatcagcccggcgaacacgccggtcccgtcgcccgccaccgagcgcgggtcgatcccggcgcgctcgaacacctcccacgcgacttcgagcagcagccgctgctgcgggtccatcgcgagtgcctcgcgcgggtgcaccccgaacagggcggcgtcgaacagggcggcgtcggccaggaacccgccgcccgcgcaggcgccgcgcagcgcgtccaggtcccagccccggtcgaccgggaacggcgacaccacgtccgcgccggagcgcacgacctcccacaggtcctcgggggaggcgacgccgccgggcagcctgcacccggcggcgacgaccgcgatcggctctcccgagcgctcgacggggcgctcgacggcgcgcgcgggcgcgaccggggtccgcgacgcgccgaacagctcgtcgtgcagcagggcggcgagcgcggtcggggtggggtgctcgaacgcggcggtcgcgggcagcgcgagcccggtcgcggcggtgagccggttgcgcagggcgaccgaggtggcggaggtgaagccgagttcggcgaagccgcgctcgtgcccgtcgaccaggaactcggcgggccgcagggccgcggtctcggcgcgcaccaggtcgagcagccgccgcctgcgctcccgctccccgcgaccggccaggctcgcccgcagctcggccacccggaccggctcgggttcggcggcgaccacgtgcagcaggtcgcgcagcggcgcgggcacgtccccggcgaccaggtcggggtcggggcgcagcaccgagacgaccggctcgtcggcgccgagcgcggcgtcgaacgcggccagcccgtcccgcagcggcagcacggccagccccggccgggcctgcgcggtctcggcctcccacggtccccaggccaggcaccgcgcgggcatcccggcggcgcggcggcgcagcacgacgtcctccgcggcggccgagtcgaacgcggtcgcgcacaccaccagctcgacccgcgcgccgagcaggcggggcagggcctccgggtccccgtcgccggtgtgcacgagcgcgcccagcggggacggcgcccggctgagcgcgcggtcgagcgcgtccggtccggcgacggcgagcacgacccgcgcgcccgaggccacgaggtcgtcgcgcagcgcgcccgcgcgggctcgcccgtggggagcggtgatcagcagcaggtcgcgcacgccgtgcgcgccgcgcaggtgccgggccagcgccgcggcggtcccgctgtccgccccggtgacggcgacggcccggcgggggtcgagcgcgccgcgcccgacgacggagccggggacctcgtgcaggcgcggcacgagcacgaccccggagcgcacgagcagccggggctcaccggcggcgagcagcccgaccgggtcgaccgcgccgtcgtcgtcggcgtcgaccgcggtgatcccggcgcggcaggcggcggtccacgcggcgagctgcccgagccgcaccggcccgccgtccacggccaccgcacctcgggtgagcaccaggtcgggacgaccggacagcgcgggcgcgtcgtcgccgcgcaggtcgagcaccaccggcgcgccaccgggagccggggcggtgggctccgccatgacggccaccggatgcccgcccggccgtgcggccgccaccggggtcgacgcctcggcgggcgtggtcgcgggcgctgtcgcagcagcgggcgtggtcgcacgggcaggaccggtcgcggcaccggcaccggtcacagcactggacgcggtcgcggcaccagacccggccaccgcaccgggcgcggtcgcgccaccgggcgctgcggccagcggcacccagtcggtgcggaacagcgccccccgctcaccgctcgcccgcaaccgcgccggcgtgtccagcagcaccctccgggtgatcttcccggaggccgtgcgcggcacggactcgatctggtacagctcctcgggcaccttgtggtacgacagccgctcccggcacaccgccagcacctgcgccgggtcgaacccctcccccggcaccaggaacgccaccgggacctcgcccagcacgtcgtgcggcctgcccaccacggcggcgtccgcgacgcccggcacggcccgcagcaccgcctccacctccgccgggtgcaccttctcgccgccgcggaccacgacgtcgtcgatgcgcccggtgaccgcgtggaacccggactcgtccctggtcgccaggtcgccggtgcggtaccagccgtcgcgcagcgcggcggccgtcgcctcgggccggttgtggtagccgaccatgacgctcggcccgcgcacccacacctcgccctccgcgccggtcggcgcgtccaggccggtgtccgggtcgaccagccgcaccgccagacccggcaccgggaggccgctggagccctcgaccctgggcccggtcggccagttcacggtgatcgacccgcacgtctcggtgctgccgtaggcgtcgatgagcggcacgccgaaccgctgctcgacggcggcccgcagctcggccgtggtgatcgcgccgccgaccagcccgacccgcaggtcgggcgcgagaaacccgcgctcaccggccgcgcgcaccaggtggtggtaggtcgtgggcacgcccgcgagcaccgtggagcgctcctcgtgcagcgcggccagcacgtcctgcggggagcgcccgtcgaccacgcgggtggtggcgccgacggcggtggtggccagcaccccggcgatgtgcgacaggctgtggaacagcggcagcggccacagcacccggtcgtccggggtcagaccgagcacgggcgcgtagcaggcggccaccgaccacaggcagttgcgctgggtggacagcacgcccttgggggcgccggtggtgccggaggtgtagagcatccacgccaggtcgtccagctccagcgcgtcgtgcgcgggcagcggggcgtcgacgcccgcgagcgcctcgtagtcccggtcgacgtgcacgacctccagcgcgggccgggtggcgcggagcccggcgagctggtcggcgtgcgcggcgtcggtgaggacgagccgggcgccgctgtcgtcgaggagggcgcccagctcggcgtcggtggacaccgggttcagcggcacgcccaccgcgcccgcccgcaccagcgccaggtagcactcgacggtctccacccggttgcccagcaggatcacggcgcggtcgccgggcagcagcctgcgcgcggccaggtgcccggccagcctgcgcgtgcgcagctccaggtcgacccaggtgacggcgcggcgcgcgtcgcggaaggcgacgcggtcgccgcgctcggccgcgtgctcgcgcagcagttcgggcaggggacggatcaggtcgcttcgcagcatctctcccacatccggttcgggggcccggtcatccgcgcagccacccaccttggcccgtgcgcgacgcgggcgcacgggcctggctagcccggacgggggaaggaattgccgccggattaccgaccgcgccccacccggtggaaggggacaagcggtggtggcggctccggtgggtccgagggggcgcgcagcgccgtcccagggccagcggcggcctcacccgacgccgtcacgcccgtggcgcgcgggcagccgggtccgggccgacgacagcggtggctctcggccggggtgggcccgacccccccgggcggggggacgggggccgcccgcccggaggtgggcctgcctccggggccgccgccccggcggggcagcccggccgagagcccgccccctcaggcctcctccagcgcgcaggcgacggcctccgccaccagcggcatctgctcctccgcccccagcagcacccggtggtgcagccacacgcagtcgctgctgagcgcttccgtgaccgggcaggcgcgggcgagggcgtccaccgtggtgtcgggggcggcgcgctcccagaacgcggcggtgcggtagatcgcgcggaacccggcgaacgccgggactccgcgctcgaccagcgcgtcgaccaggcgggcgcggcgctcctcgccgatgccggggacctggaacatggccatgtagtgcgggttgcggtcgcagcgcgggtcggtcccctgcgggacgacgccgggcacctcggcgagcagccgcgacagcacgggccagtgccgctcgcgcacgtcggtctgctgctggagcctgccgagctgggcgcgcagcagcgcggcggagaactcgttcatgcggaagttcgacccggccgtgcggtggtggtaggcccggtcggcgagcgggcgcccgcaggcgtggcgcaggaacgcctcctcgtgctggtccccgtccgggagcagcacggcgccgccctcgcccgcggtcatcagcttgccgttctggaagctgaaggcggcgatcgagccgagcgcgccgaggggctcgccgcgccaccgcgcgccgtgggcgtgcgcggcgtcctggagcagcgggacgccggtctcggcggacagggcgcgcagcgcgtccatgtcggcgagctggcccgccatgtgcacgggcatgatcgccctggtgcgctcggtgatcgcggcccgcacggcggccgggtcgacgcagtaggtgtccgggtcgacgtcgacggggacgacgaccgcgccgagccgctgggcggcctgcgaggaggagatgaaggtgaacgcgggcacgatcacctcggtgccaggcccggcgcccatgacctccaggacgagttcgagggcgtgcgtgcccgtggtgacggcgagcgcgtgccgggcgccgtgggcctgcgcgaactcccgctcgaacgagacgacctcgtccccggtgatccgccaccaccggccgctgtcgagcgtgcgcagcagggcctcgcgctcggtcgagtcgaactgcggccaggcggggaactccagcttctgcggggccttcaggtgcacggtcggtcgccctcgtctctgctctcggtccggcgcgctctccgggggccggtgcgcggcccgaagctaacggcggccggaagagccccccaaccccctgacgcccccctaggtcccccctgcgttcagccggttcgccgcggtccccaccaccggggtcaggccgtcagcacaatcgcaccgtccactcacgtctttgcgctctgcgaggtgttcctggtgcccgacctgctcctgctgaacggcccgaacctgggaatcctgggccgccgcgaacccgaggtgtacgggaccgcgacgctcgccgacatcgaggacgcggtggccgacgaggtggcggaccgggggtggaaggtggtggcgaggcagcacgacggcgaagccgagatgatcgggaccatccaggacagctacgacacggtgggcgctatcgtgaaccccggcgcgctcatgatcgccggctggagcctgcgggacgccctggcgaactacccgagcccgtggatcgaggtccacctgtcgaacgtgtgggcccgcgagccgttccgccaccagtccgtgatcgccccgctggcgacgggcctgatcgcgggcgtgggcgccctgggctaccgcctggcggcacgggcgctgctggaccgggtcgggggttgacgggtaggtgttggttggcgggcgagggctggctgggggacgggcgctgctggaccgggccgctggcgagcgagtgttggttggcgggcggctggcgagcgggtcgactgaggggcaagccggttggtggcctgaccggcggctagctccgcagttccgacgcggtgggcccacgtcctggccaaccggcgggctgccctggccaacgacgtcaccgcgccgccacgtcaccgcgctgccacatcgccgcccccgaccagcgcgctactcccggtttcccccacgcgccaggacaagcgccccgtgcagcgaggactcggccccgagcaccgccggtcgcaccgatggcaccgggtgcccggtcctgcccagcccggcgacccgctcggccacagcgggcacgagccctggcagcgcggtggtgacaccaccgccgatcagcgccaactcgggccgaaccagctcccccaccccgatcaccagcgcggccaacgcggacgcggcgtcctcacaagcctccaccgcccaccgctccccacgcgcgaccccgtcccggacgtcccccagcccaccgggccgcccaccacgcgcgacggccccgcgcagaatcgcgggcccggacgcgaacgcctgcacgcacccccgccgcccgcagtcgcacagccccccaccccgttcgacgaccacgtgccccacctcgcacgagccacccgagttcagcggcagtccacccgagacgatcccgccaccaaccccggtgcccacaccgacgtacagcagctcaccgcacccggcggcatcggcctcggcaagcgccgcgaggtcaccgtcatccccgtgccgcaccgaggcgccggggaacatcgcccccaacgcaccgagcacgtccaccccgacccacccgggccgacccggccaggcgaccacgacgccggaccgatcgagcgtggcaggcacagcgacaccaaccgacctgacacccccacccccaccagcccgctcgaccgcccgaacgagcaaggcgaggtcttcgccagcagaagcgccgctctcccaccgcaccacgctgtcgcccgaccgcccgtccgcaccaacaacccgaacagcggccttcgtccccccaagatcaactccaaccaccccgccagtacccgaaccgctcaccccacccccctgatcccggcgcacagcagcccaccccgtgttcccggcccacgaccgccagcaagcaaccccagcgcaggctgtccggtcaggagctagtccgccgctgtccgcacactgggcacccgcaggccacccgctcgtcgtgaagccgcccgctcagcagccacccgccgggcccggtcgttcgtctccaccagcgcgaaccgggccgtctccacagcggccctggtagcgcccagtgcttcgcggcaggactgcgcaccccgaccttcgagcgcgccgcccccgtcctcggcgccccgctgctcccacgcctcgcacagcagcacgaacgcggtcaggcaggacgtggcggccacgaccagcggatcacgtccctcgcccaccagcgccaaccgagcgacgatccgctccacctcggccttggccagcacaggccccagcacctctgcggccacgccacgccctcctccacaccgaacccggcgcccgaaccaccacgcggcgacgccgtcagcagcaacctcaccaacaccactcagcacacaccgggccatgcccacaccgagcagcgccgcacccacgccccgcaggactgcgccgtgccacacacgccgcagccccgcgcacgagccctaaacaggccccagcgtgaagtaccggtcggtccacaggtcgaggacctcctgtcggagggcgtcgtccacctcgccgtccaccagttcggccaccgcgctcttcccgtccacccgcgtcagcagcccgtgcgcccgcgccgagatcgccttgcgcggcccctccgcgtagtccaggaagacctcgtgctcctccacccgaccgccaccgaccgggaaccgcacccggcccaccagcctggtctcggggcggaaccggatcaccgcgtcgtccagcgcgcccaggaagtccgggcgcctgcggaccaccgagtcgccgaccaccaggtagtccagctcggtggtcaggaagctggtcagcacgtcctcaaaggtctgcacgatcggctcggcgtggttgttgaacgaggtgttcagcagcaccggcgtgccggtcagctcgccgaaccgctccaccagccggtggaacgccgggttggccgtcgaggtcaccacctgcacccgcgcggtgccgtccacgtgggtcaccgcgcccaacccggcccggtgctcgggccgcacgggcaccacgaacgacatgaaccccagctcggcctcgacgtccgggatctcgaagtactcggcggcggcgtcgggggtgaccacgggcgcgaacggccggtagctctcccgcttcttcaccatcgcgttgatcctggtctggttcgcggcgggccggggatcggcgacgatgctgcggctgcccagcgcgcgcggcccgaactccgacctcccgcgcgcccacccgagcaccgcgccgtccgcgaggagccgggccgaggtgtccaccaggtcctccaggtccgccgcccggtgcaccagctcgacgaacccgtcccagcccttgagcttgagcacgacgtcccgctcgtccccgagcccaccaccgaggtccgccctgcgcagcgccacgcgcggcacccgcccggtcagccggtgctccaccgccagggcggccccggtcccggcgcccgcgtcgtgcgaggcggggtgcacgaacacccggtcgaacagccccgaccgcaggatcaccccgttgaggctggagttgtgcgcgaccccgccgccgaagcacagcgcgcgctcaccggtccgctcggcccagtgccggatcacgtgcagcgcgatccgctccacggtctcctgcagcgcggcggcgaagtccttgtggtcctgggtgaactcctcgcccttgcggcgcggcgcgaacccggtgtcgagcatcgcggggaggaaggcgttgggcgtgaccacgttgcccagcagctcgtactcgccgccctcgcccagcgagtgcagcgactcgaacaccgaccggtagcgggccgggtcgccgtagggcgccagccccatcaccttgtactcgtcgccgaagccgtagccgagcagctgcgtgccgaacaggtaccagaaccccagcgaccggtgcacggggtacgaggccagcgactcgaccccgccgggcccgatccggtagacggtcccggacgcgttctccccccggccgtcgaacacgaccgtcagcgccgactcgaacccggagtgcccgaaggaggacatcgcgtgcgccaggtggtgcggcacgtagtgcagcttctccgcgggcaggtcccagcccagctcgctgcgcagccggtcgcgcaccaggtcgcgcgagtaccgcagcggcacggcggggctctggcagtagagctggccgagcaccttgtccacgtggtcctcggggaagtagaacgccaccgcgtccacgtcgtcgggccccagcccggtctcctcgaagcaggcccgcacggccgcgctggggaaccgggtggtcttcttgatccggttgtaccgctcctcctccacggcggccagcagctcgccgtcgcgcgccaggctcgccgacgagtcgtggaagaagaactccagcatccccggcaccagctcctcggcggcggtcgagaagttgccgttcaggccgagcacgagcatggtctctgtcaccgggccaatccgtgtgggagggacgagcggagaccgggtcgcgggactccacttcggacggtagggcacgcgggcgccgttggcagttgccgcttccacggctggcgaacaaagcggtaatcccttgcgccacaggcgattgacaccccggcgaggccctggtttcactgctgtccgcacgtcaccggcgcgaggaggcccccgtgaagtacatgctgctcatccacaccagcgacgagcggtggcagaccctgacgcaggccgacaaggaggagttcctggccgagtacgccgcgttcaccgaggagatcgagtccaccggcgagcactgccacggctccccgctgacccaccgcaggaacgcgctgtccgtgcgggtgcgcgacggccgcgccgccgtggagcgcggcccgctcaccgacgacgaccgctacctggccgggtacttcgtggtggactgcgccgacgaggaccgcgcgctggagatcgccgcgcgcgtgcccgacgcgaagctgaacgtggtggaggtcagggcgctcgcggtggtctgacgcgcccgcgggctcaccccgccgggtccggggcgggcgaccgcctcggccggtgggcgcgccgcagcagcggacgctccacccaccggtgcagcagcaggctgagcccctgggtgagcgcgtacagcgccgccaccagcagcagcccctcccccagcccgagcagccggtcccccgccacgtgcttggcgaccgcgagcagcggccagtgcagcaggtaggtcgcgtagctcatgtcccccgcgacccgcacccaccgccgggacagcacggaccgccgcccctccaggtcccgcacggcgatgacggggacgagcagcgccagcgggaccgccgcgatcgcgctgcgcgcgtacacctgcggcagcaccggcagcagcgcccacaccgccgccacccccagcagcggccaccacgcccgcaccggccgccaccgcccggtggccacgatcctggcgagcaccacgcccatcaggaactccggcaaccgggtcagcgggaagaagttcaccagccacgcctgctcgaacgacagcggcggccacagcgcccacggcggcccgtccaccagcgccccgatcacgcccggcagcgcgagcaccaccgcggcgagcacgccgaagcacgcccacaaccggtggtcgggcacccgcagcagcggccgggacagcagcggcagcagcaggtagaacaccacctcgcaggacaccgaccagttgatgccgttgaccgacagcaggtagtcgggcagcggcacgagcgactggagcagcgacaggttgaccagcgcgggccccacctcgggcagcggccccagcagcggcagcgggccgagcgccgccaggagcagcagcaccgcggcccacgtcagcacgtgggtgggcaggatgcgcacgacccggcgtcggaagtaggggcccacgccctcgcgccacggctccgaccacaccagcacgaacccgctgagcacgaagaacaacgacagcgccgcgggcccgtagggcaggaggaacccgagcgagttgaccgattcgtcggcgaagaaccgggcggagccgagcgcgtgggccacgaacacgagcagggccagcggcgcgcggatgccggtgagggagggaagccgaggagggaggacgggaccgggggtcatgtgctgggctccctgtctgtggggatcagcgcgcgtgctgccggggttgcgggagcaccggcgagggctcggtcgggcggcacccgcccgacctgagcccgtggtggaccagccgcagcgcgtcgtgaccgggcacgcgctccaaccagtcggccgctcgccggtgcttctcggcgcgggcgtgctgggggatggtcaggtaggcgacgtcgcgcacgagcgggctgcggaacgcgtaggcggtggcgcgctcggcgtcccggtcccggcgcaggacctcgcgccgctccaggtgctccagcgcgcgcgccacgtcgggcacggcgccgccgccgagcgcgcgcaccccctcctcgcacaggtcctcgccgaggacggccgcgtcgagcagcaccgccttggcgtcggcgggcaggctgtcgatgcgggccgccacgacccggcgcaccagcggcggcagcggcggcgactgcccgccgcccccggacgacgggcccgcgtcggcgagcaccctggcgtactcgacggcgaacagcgggttcccgccgatgtgggcgatcaccgcgcggtaggcggcgctcgcctcgtcggtggcggggccgcgccggacgcacagggcggtgagcagccgctccacgtcctggtcggcgagcgggtcgagggtgatcgtgctggcctcggccttgccgccgccccaggcggggcgccggtcgaacagctcggggcgcgcggtgaccacgaccagcagcggggacgacgtggcctcggcggtgaggtggtccacgaagtccagcagcgcctcgcccgcccagtgcaggtcctccaggaccaggacgagcgggtccgcgccgccgaggtcctccagcacgtgccgccacgccgcgaacgagccgtccgggagcacgtcgggcacgtcgagcccgaacaggacgcgcaggctggccagcacccggtccgcgcgctcgcccgcgccaccgctgccgcgcgcggcggcggcgagcttggcggcgaccagcccgaccgggtcggccgggccgatgcccatgcgcggggcgagcgcgtcggcgagcggggcgaggtcgccccctccgccgaggcgggggcagcggaccacaacggtcgccgaggtctcggccacgcgctggaactcgtccacgagcctgctcttgccgatgccgggctcgccgagcacggtcaccaggtgcgtccgggacctgcggccgacctgctccagcaggccgcgcagcagggccagctcgcggtcccggtccaccagcggcggcgtggccgggggcccgcccgcgccctgcctggccccggtggcccaggacccgcgcaccgggtcgtgggggccgtcgtagccgatggcgccctcgctggcgcccctggtctcctcgcacaccctgacctcgccgggcgagacgtgcgcgagcagcctcatgcagcggtcgagcacgccgccgccgacctcgggcgcgcgccctgggtggcgccgcaccagggcgtgcccggtggcgacggcggcggtgacgcccagcgcggcgggcccggcgccgcgcggcccgtgggcgagccggtcgcgcacggcgagcgcggcgcgcaccgccctggtggcgtcgtcctcgccgctgcgggtggagccgaacagggcgacgatcagcgacgcggccttggcggtgacgacgccgcccgcccgctcgacctcctcgcgcaccgcggtggcgacgtcgctcagcgcgtcgtccacctcctcgggcggggcggcggccacgtcgtgctcgaaccgggtgaggaccatgaggacgctgacgtgcttgagctcggcggtcggcgcggacgccggggggaggggcgcgagcgcgacgggggcaggcgcgggcgccgggggcgcctgccgctgcgggacgatcccggccgggggcgccgcgaccgggggcgccgcgaccgggccaccgccctcggtcgccccggaccgcgcggcgggcgccgcctcgcgccgggccgggagccggtgcggcacctcctggttgaggatcgcccgctccagctcgcgcagctcgcggccggggtcgagccccagctcgtccaccagcctggccctggtgcgccggtacacgccgagcgcgtcggcctgcctgccgcagtggtagagcgcccgcatgagctggccgcacagccgctcgcgcagcggctcggcctcgacccaccgctccagctcgtagatcatctcgccgtagcggccggacgcgaactccgcctcgacgcagtcctccagcgcggccagccgggtgttgcgcacggccgccagctcgggccagtcgacgccctgctcgaccaggtcggccagcagcggcccgcgccacaggccgatcgcgtcgcgcagggtgcgggcggcgcgctgccaggagcccgccgcgaggtcggcgcgcccggcgtggacgagcctgcggaagcggctcaggtccacgtcctccgggtcgacgcgcagcaggtagccgggggtgtgggtgagcagggcgggcgcgccgggcgcgtcaccggcggcggtcagcacgccgcgcagggcggcgaccgcgttctggagcatcttgcgcccggtgggcggcatgtcctgcggccacagcgccttgagcaggctgctggtgggcaccacgctgttcgggtgcagcagcaggtagccgagcgcggcgcgctgcttggtgccgccgagcgccaccggtcgggaccggtcgacgacttcgagcggacccaagagctgaaagcgcatcactggtctccaaggcgcgtggttgctccgggtcgcttcccgccgctgcgctgggacggttccgctctggaccgtcgggacggtacccccggccatcgcgcggccgggagttcgcagcggctttttccccctaccgtatgtatcggaacgctcaccgtgcgcactggtgcggagagctcagcgcgtgcgacgggccagggtcatgccgtcggcgtgcgcgagcagcacggcctggacccggtcgtcggcggcgatcctgcggttgagctcccggacgccctcggtgtcggggtccaccgcggcggggtcggcgacccggccggagaacagggtgttgtccacgacgaccagcccgccggggcgcaccagctcgacggccagctcgtagtagcgggggtacccggccttgtccgcgtccacgaacaccaggtcgacccggcccgcgagctcgccggtcagcccggcgaggctgcccgcggcgtcgccgacgagcacctcgatccggtccgcgacgcccgcgcgctcccagtgcggccgggcgatcgcgggccacttggggctcagctcgcaggtgatcacccggccgccgtcggggagggcggaggccaggcacagcgtgctgcacccggtgaaggtgccgacctccacgaccagccgggccccggtgagcgcgaccagcatggcgaggaaagcgccttcccccggtgggacggggaggacgccgccgccgggcagcagcgcggtctcggcgcgcagctcggcgagcgccgccggttccggctgggagacctcgcgcaggtagtcgagcagcgcgggcgtgacgacgacctgctcacccacgggtgggcacctccagcagcgacggggcttcgacggtgaccgcgcgcggggcgggtcggggcgcggggcgcaggtgcagccgccgcaccgggttcccgctggcacgcgcctcgggaacctcggcgacggggacatcagcggcgcggggctcggcggtccggggctcagccacgcgagactcggcagcgcggggctcagccacgcgagactcggcagcgcggggctcagccacgcgagactcggcagcgcgaggctcggcagcgcgagtctcggcagcaccggttcctggagcaccgcccgcgggagcaccgcccgcgggagcaccggctcctgaagcaccggctcccgaggcgccgatccccggagcgcccgcgcctggggcgcctgccgcagcgaggtgttcggggccgccgcccaccgccgccagctcggcgcggcaggcgcagtcggcgtcgtcgtacccggcgaaccggtaggcgatctccatcatgcggttgcggtcggtgcgccggaagtcggccaccaggtgcgcgcccgcctccagcgcctgcccgcacagccagtccagcagcacgctgcccacgccgtagggcaccacccggcacgaggtcgccagcagcttcaggtgccacgcggcggtgtgccgctccagcagcacgatgccgatcgcgccgtgcggcccgaaccggtcggtcaggcccgccaccagcacctcgtgcgccgggtcgtccagcagggcgcgcaactgcccgtgcgggtagtgcacgccggtcgcgttcatctggctggtgcgcgcggtcagctcctcgacccggtgcaggtcctcgggcctggcccggtcgatccgcagcaccagtcccagcgagcgcaggaactcctcgtcgggcccggtgaacgcggcctgctcggcgccgcgcgcgaacccggcccggtagagggcgcggcggttgcgggcgtcctcggtgacgacggcggggctgaactcgggcagcgagcacagctccagcacgcgctcggccgggtagcagcggacctcgggcaggtggaaggcgacctcggcgcgctcggcgggccggtcgtcgacgaacgcgatggcgcggtgcgcgaagcccagccggtcggcgatgcgggcgacggcggcggacttgacgccccagccgatctcgggcagcacgaagtactcggcgacgcccagcgcctccagcgcggcgagggcgggctcggggtcgttgcggctggccaccgactggagcacgccgcgcgcgtccagctcgacgatcgcggcgcggacccgctcgtccagcacgggcgcgtcgccctcggcgagggtgccgcgccagagcgtgtcgtccaggtcccacaccaggcacttgaccaggtcgccgctcacgccgtcgccaccgcgtgctcggccagcacgagcctgcacagctcgttgctgccctcgatgatctccatgagcttggcgtcccggtaggcgcgcgcgacggggtggccgtcgtgggcgcccgccgaggcgagcacctggacggcggtggcggcggcgcgcgccgcgtgccccgcgctcacgtgcttggcgagcacggtggcggtgaccagctcggccgagctctcgtcccagcggcggctggcgtgctcgcacaccctggtggagacctgctcggcgaccagcaggtcggcgaggtggcccgcgacgagctggtgcgcggccagcggcacgccgaactggcggcgggtgcgggcgtgctcggcggacgcggccaggcaggcgcgcaggatgccgacgcagccccacgccacggacagcctgccgtgcgcgaggaccgtggtgaccagcagcgcggtggagacgccgtcgccgcccagcaccgaggtggcgggcaggcgcacgtcgtccagctcgacgtcggcgtggcccgcggcccggcagcccatcgggttggcgacgggggtgatcgtgacaccgggcgcggtggcgggcaccagcgcgaccgccgcgccgtcgccgcgcctgcccgccacgaggagcaggtcggcgtagcgggccgcggtggtccacaccttgtggccgcgcaccaggaccgtccccggttcgccggggtcgtcctcgacggtggtggccatggcggacaggtcgctgcccgcgccgggctcgctgagcgccacggcggccagcgagcccccggtgagcctgggcagcacggcggcgcgctgcgcctggtcgccgaggcggcgcacggtccaggcggccatgccctgcgaggtctggacgctgcgcaccgagccgcacagcgcgccggtgcgggcggtcagctcgccgttgtccaggctgccgagcccgaggccgccgtgccgctcggcgacctcaccgcacagcgcgcccgccgcgccgagctcgcggagcgcggccaccggcagctcgcccgccaggtcccactcccccgcccggtcggcgaccacccgctcgaccagcgcggtcgcctcgtccacccggtcagccaccggcgccgcccgcgcggagccggtccacgagcgcggtcatggcgcgcaccgtgcggaagttgtccagccgcaggtcggggcccgcgatctcgacgtcgaagcactgctccaggtgcacgaccagctccatcgcgaacagcgacgacaggccgccctcggcgaacaggtcccgctcggcgtcccactcggcgccggtgcgggcgcgcaggaagtccagcaccgcgccctggacgtcgtcggtccgcgactccgtgcccgaggtcatcgccccgtcccttcacggccggggtggccgtagtcgtagaagccccggccgctcttgcgcccgaggtgccccgcgtcgaccttggccagcagcaggtcgcacggctcgcaccgctcgtccccggtgcgcctgcgcagcacgcgcagcgagtcgacgaggttgtcgatgccgatcaggtcggcggtgcgcagcgggccctcgcggtggccgaggcagtcgcgcatgagcgcgtcgacggcggcggcgtcggcggtgcccgactccacgacgcgcgccgcgtcgttgatcatcgggtgcagcaggcggctggtgacgaagcccggcgcgtcgccgacctgtaccggggcgcggccgaggcgcgcgagcagggcggtgagcccggcgaacgccgcgtccgaggtcagcgggccgcgcgctacctcgacggcggggatcaggtacggcgggttcatgaagtgcgcgcccacgaggtcgccgggcctgggcagcgcgggggccagctcgcccatggggatggaggaggtgttggagaccagcggggtgcccggcgggacggtggcgcagaccccggtgagcgccttggccttggtctcggcgtcctcggtgaccgcctcgaccacggcgaccacctcgcgggggccgcccgtgtcggtcgcggtggtcagctcgccgagcggcccggcggcgacgccgagcagctgggcggtccgcaggtgcgcgcgcacgtcggcgcgggccgcgtcgagccggtcggcgtccgggtccaccaggagcacgggcaggcccctggagagcgcgagcgcggtgatcccgcagcccatgacgcccgcgcccagcaccagcagcgggcgctccccggccgggcggcccggttcgccgggcgacgcgccgccgcgcgccgcgccgccgggcgacatgccgccggaggacggaccgagggacgacgggccgtcgggtggcgggccgtcgggtgacgggctggcgagcgctcgggccggtgggtcgtcggtgtcgccgggcacggggcctccgaggggctggagctggggaccgggtcggttgtggacagtcgagcaccgcgcgaggccctcccgcactggtcggctccaccggacgcccacaccccggcaaccagctcgccggtgggcggttcgttacccggtggtggaaccgcggattgccgctgcccggcgggcttcctagcgtgggctgcggatcgtcaccgccgcgcggggaaccggcttcgcgccgcccgacggccggggaggtcgtggcctgatgctcgacgcgatcgtgatgggtggtgggcccgccgggtccgtctgcgccgcggtgctcgcccggcagggccgctcggtgctggtgctggagcggcaggagttcccccgcttccacatcggcgagtcgatgctgccgtacatggtgggcctgctggagcggcacggcctgctcgacgcggtgcgggagcagggctacgtcgtcaagcgcggcggcgagttcatcgacccgaccggcacgaagttcttccgcgccggggtgttccgcgccgacttcgccaagaccggcgacggccgccaccacgagacgttccaggtcgagcgctcgcacttcgaccgggtgaacctggaccaggcgcgcgcggcgggcgcgaccgtgcgggagggcgcgcaggtcgtcgggctgctggaggagggcggccgggtggtgggcgtgcgctaccgcgagggcggggtcgagcgcgaggagcgcgcccggtacgtggtggacgccaccggccgggcgggcgtggtcgccaaccggttcgggctgcggcggatgatcgaggacctgcgcatggtggcggtgttccaccaccgcgacgggctggacgaggcgcacaaccccggccacgagggcgacatccaggtcggcagccactcggacgggtggatctgggcgatcccgctgtcggcggaccggatcagcgtcggcacggtcatgcaccgcgaccggctgcgcgggcgcaccccggccgaggcgttcgccgagcacgtcgagcgggtgcccaggatcaaccagcggctcaccggcacctccgcgacctcggacttctgggtggagaccgactacagctaccactccgaccaggtcaccggccccggctgggtgatggtcggggacgccggctgcttcggcgaccccatgttctccggcggggtgctggtgggcatggccacgggcgcggaggccgccgaggcgctcgggcgggcgctcgactcgcccgccgacgaggagcaggcgctcaccggctactcgaacttcttcaagaccggctacgacacgtacgtgcggctgatcttctccttctacgagggcgagctgctgcccgcgctggccgaggcgcactcggaggcggggaacctctccgaggcggacatggagatgtacgtggtgcggctgctcggcggcgacttctggagcgcgcgcaacccggtggcgaacgcgctgcgcgcgaacccggcgtggtcgacgttctcgccgttcgagccggtgcaccgctgcccggtgtaccccgagctggacgtggcggagctgggcgggctcgcgggcacggtcggccggtgagccgggacggggtggtcgagcgggtcccggtgctggtggtcggcgcggggccgaccgggctggcgctggcgtgcgggttgcgctcgctcggggtggaggccctggtggtcgaccgggcgggcggtcccgcgaccacctcgcgggcgctgggcgtgcagccgcgcggcgtggaggtgctgctgcggctgggcgcgctgggcgacctgccggggctggtgatcccgctgcgcgaggtgcggatgcacagcgagggcaggctggtgtcggtgatgccgatggcgcgcatggcgcaggcgtcggggcgcgcgccgctgctggtgtcccaggtggaggtggaggcgtcgctgcgggagcggctggccgcgctgggcggcgcgccggagtggggcgtggagctgctggactgcgcgcaggacgcctccggggtgaccgcgctgctgcgcgacggcggcggtgagcggcgggtgcgggcggactgggtggtcgggtgcgacggcgcgcgcagcctggtgcgggagctggcgggcatcgggttccccggcgagcaggtggccgagcacttcctgatcgccgacgtgcgcgcggactgggcggtggaccggggcgtgatgggcagctggacgcgcgggcgcgacatggtcagcgtgtgcccgcttcccggcgaggaccggtggcggctcatgggccccgcgcccgccgggacggccgggcggctgcgcgacgacgaggtgcgggagctggtgctgcgccagctcgcgcgctgcacgccgcaccccgccgaggcgatcacgcgggtggagtggctgtcgtcgttccggatccaccgcaggctcgccgaggcgcaccgccgggacaggctgctgctggccggggacgccgccgcgatccaccacccgttcggcgggcagggcatgaacaccgggctgggcgacgcggagaacctggcgtggaagctggcgctggtggcgagcgggcgggccgggcaggagctgctggacacctacgcggccgagcgcaggcccgtcgcggcgggggtgctctcggggaccggggtgaccaccggcctggtcgcggcggacaacccgctggtggcgctggtgcgggaccgggtggtgatgccgctgctgaacacccggctcgcgcaggggttcctgtggcgcagggcctcccagctgggggtcgggtaccggcgcgggccgctggccgctccgggcgcccggcggggatcggggcccgtgcccggcgaccgggtgcccgacctggcgtgccacgacgggggcgggcggacgacgctgcacgcggcgctgcgcggccggtgggcggtgctggcgccggacggggtgggcgggtcgaccgtggaagcggtgcgggggctgctcggcgtggacgccgtggtggggctggcgccggtggcgggcgggttggcggaggtgctgctggtgcggccggacgggcacctggcgtggcgcgggcggccggaccccgagggggtcgcggcgtggctggggtcggcgctgagcctggtcggggagggggcgctgcgatgagcctcccagcggactcaccaccgccggggccggtggagatgcccgacgagagctggacgtcgctgggcaacgcgggggcgcgggcgcaggagtccgcgcgggccgaccggttgttcgacgacccgctggcgcgggcgttcctggacgcggcggggacggggaacccgctgctgcccgaccggggtggcggtgatccggggctgctcgggcagatcacggacgtcatcgtggtcaagacggtgttcttcgacgcggtgctggcgcgggcggcggcggccggggtgcggcaggtggtgctgctggccgccgggttggacgcccgcgcgttccggctgccctggcccgagggggtggtggtgttcgaggtggacctgccggacgtgctggggttcaaggagcgcgtggtgcgggaggtcggggcggagccctcgtgcgaccggcgggtcgtcgcggcggacctgcgctcggactgggtggccgcgctggtcgccgccgggctggaccgggaggccccggtggcctggctggcggagggcgcgctggggctgctggacgaggccgggtgcgaggagctgatggcggcggtgctcggggcctccgcggcggggagccggttcgcgctcgaccacacccacgacgggtggaaggccggggaggcgctgggcgggtacctggaggggaccggggtctgcttggccgacctggtgaaggggggtccgcgggagccgggcggggcgtggttggcgcggcacgggtggcgggtggcggagtacgacgtcgtggcggaggcggcacggcacgggcggcccgcgccgaccctgttccgggtgccggagcggcgggcgaacgcgacgatcctgttcgaggccgagctggggtaggcgagcgggaggcgggccgggcggggacgggcagggcgggacaaagccgggcgggacaaagccgagctgagcagcgccgagccaagcaggacgagccaagcaggaccgggctgggcgggacagggccgagccccggcccaccttcgcggcgtggtccacctctgcggcgtggtccacctccgtagcgtggcccggctctgcggcgtggctcgcctcctctgcgtgacccggctccgcggcgcggcccggctcagtcggcgcagatcccggtgcggatctcggcgctcacgcgcgcgtgctcggccgggtccagcagggtggtcaccgtctcgtggccgtcacggaccacgaggtgccgcttgcccaccagggcccggtggccgtcgtcgaccgcgcggcacagcagggtggtgcccgcgatcaccgcgcggtccaggtagcggcgcagtctcggggtgaagccgtcccagtcggacagcgtcctgggggtggtggcgaactcgtagagcgggcgccagtcggtggtgcggctcctgcgcagcacgaggtgcctgccgtcctgctccaccacccggaacgcgcacccgtgctggacctgctcgtccggggccagcctgatcgggtccaggtaggacggtccggcgaagcccacgtcggccagcagcacctcgccgtccaggtgcacggcggtgaaccggtgcgccaggtcggggctgaagccgccctcctcgtcggccacgcccgccgacagcacggtcgtggtgaagccgaggtcggtgagcagccggtggaacagcaggttcagctcgaagcagatgccgccccgcccggccgtgacgagcccgtcgaaggtcgcgtccacgtccaggtcggcgaggttgtcgggaacggggccgtcctgagtggcggcggcggtgttgtcgtagtgcagcgcccgcaggtgggcgcggtgcagcgcggtcagcgcggcccggtcggggcgggtgacctcgacgccgagccgggccaggtaggtgttggtgtcgtacacgtcagaccgtcccgctggtgtggggcgcgcgcagcgcggcgcgcaggtgggcggacagcgccctcggggtcggctcctcgtagaccgcggcggcggtgacgcgcacgccggtgcgctcggccagcaggttgcgcagctcgacggcggtgagcgaggtgaacccggtgtcccagaagggctcgtccgggtcgacggactcaggcccccggtggccgaggacggtggcggtggcggcgcgcacgagcgtcagcagcagctccggcagctcgtggtcggcggccccgcgcagccgatcgaccgggtccggggcgacctcggcgcgggcgctcctgcgccgcacgggcacgagctggcgcagcagcggcggcacgtccgcgtcctgcggcgtcgcggccaggctcagcggcgccagcagcgggcggccgtcggcgagggcggcgtcgaacagggcggtggcctcggcgtcgtccagcgggtgcagcccggcgcgggaggcgtcgaccccggcggccatgccggagcgccacggcccccaggccagggagagcgcgtgcagacccgccgcccgccgtcgtgcggccaggccgtccaggaaggcgttcgccgccgcgtagttggcctggccgggggtgccgagcacgcccgccgccgaggagaacagcacgaacagcgtcgtggtgccgtcgcgggtcagctcgtccaggtgggcggccaccagcgccttgggcgccagcacggcgtccagccgggcgggggtgagggcggacaggacgccgtcgtccaggacgcccgcggtgtgcacgacgccgccgggtcggtgctcggcgagcagcgcggcgagcgcggcccggtccgtgaggtcggcggcgaccaggtgggcgcgcgcgccgagtcccgcgaggtcggcggccagttcgggcgcgccgggcgcggcggggccgcgcctgctggcgagcacgagggtccgcactcccccggcggccaggtggcgggccagggtggagccgagcgcgcccgtgccgccggtgatcaggacggtgtcggggagggcgcgcacgccgtcgtggcggggcgcggtgcggcgcaggcgcggcacgcggagcgccccgtcgcggacgaccacctcgggttcccgcgtggtgggcggcggttcggggccgtcgccgtcctggacgacgagcaggatccggccgggttcctcggcctgcgcggaccgcaccaggccgcgcacggcgcactgccacgggtcgtcggggcggtcggtgcgcacgaccaggcggggcgcgtcgctcgcggcgaagcgctggagcacgtcgagcgcctcggcgggcgtgaccccgcgcaggtccgcggacagggcggtggacaggtcagcggacagggcggtggacgggccggggggcgcgggggcgggcagggggacctcgacgacctcgaacagcgacccccgcgtgacggcgcgcggggacgccgccactcgggtgacaccccccaccgacacgaccgggtcgcccgcgccgtcgaccgccaggagcggcgaggtcccgcgcacccgcagcgcggtcgccccggtggcgtgcaggacgacgtcggcgcagcgggcggggtgcggcagcagcgcctcccagagcgcggggtgcaggccgaagccgtcgggcgcggcgccgtcgggcagcacgagctcagcgcccggcgccggtccggcggcgggcagggcggaccgcgcaccgttggtcgctgccgccggggtgccggccccgtggggcaaggacgcgccattggctcccgccgccgggacatcagccccagcgggcggccacgcgccattggtccccaccgcgagcgcctcggagtcgtgcggtagcggttccgggtggcccgccggggcgagcacgcccgtcgcgtgcccggtccaggcgggctcgtcgtccggtcgggagtggacggtcaccgcacggcggccgtctgcgtccggggcggtcacgcggacccggacgtgccgggggcccgcgtcgagcgggaccggggtggccaggtcaagctcggccaccaccgggcagccgacctcgtcgcccgccctgatcaccggttccagcagcgccgccagcggggtcgcgcccagaccggcccggcccgcgtgccagcgcgcggacagcaggacctcgtcgccgggcagcacgtccagcgcgtccaccagcgggtgaccggtcgcggtggcgcggggtgcggcgtcgagccagtagcgggtgcgctggaaggggtaggtgggcaggtcgacgcggcgcgcgcccgcgccctcgaacgcgggccgccagtcgacggccgcgccgcgcgcgtgcagcacggccagcgcggtgagcagggcgacgacctcgtcgtggtcggcgcgcagggcggggatcgcgcgggccaggacctcggcggcgtgcggggtgagcacggcgtcggggcccagctccacggccgtcgtgacgccgctgcccgccagggcgcgcagggcgtcggcgaagcggaccgcgccgcgcgcgtggtcggcccagtggtcgtggccgacgtgggtcagcgtgcggccggtggtggtggagatcaggggtgtggtcggggtcccggtggccaggccgcgcagggcgtcgagcaccgggtcgacgtgcggcgagtggaacgcccggtccaccgggagccgcttgcgccgcccgccgaggcgggccgcgacggccacgaccgcctcggcgtcgccggagagcacgaccgcctccgggccgttgacgacggcgaccgcgacgggcaggtcgccgatcgcgtccagcgcctcctcctcggtgacctcgacggcgaccatcgcgcccggtcgcaggtcgcgcatgagcacgccgcgctcggcgaccagcgcgcacgccgcgtcgagcgggaacgcgcccgcgacgtgcgcggcggcgatctcgccgaccgagtggcccgccaccgcgtccggcgcgaggccgaacgcgcccagctgggcggccagcgcggtctgcagggcgaacgcggcggtctgcgcgtgcagggtgccgtccaggccggggcgctcccccagcgcgacggcgcgcaccgggtgctcggcgcggccgtcgagcgcgcggtcgagctcggcgcaggtggcgtcgaaggcgtcgcggaacgcggggaaccgcgcccgcagcgcccgcccggtgcccggccggtagccgccctgcccggtgaacagccacgccgtgccgcccggcgccgggacgccggaggcgacgacctcgccgtccagcagcacggcgcggtggcgcagggtggcgcgcgtggtgaccagggaccagcccacgtccaccgggtcgtgcggcagcgcggtgatccgctcgacctgggcggccagcgcgcgctcgtcgcgcgcggacaccaccaggggcaccagcggcccggacgcgcccaccacgtcgacggtccgcgcggacccgtccggcccggcgcccaccggccgcgccgccgggcccggcacgccgggcgcagccgcctccgggtcgggtgcgcgcgtcgcggcgcccaccgcaggcgccggacacaccccgccgggatcgaccgcgccccccgcctccggctcgcgcgcgcccgcggcccgcgcgacacccaccggctccgccccgcgtgctcccaccgcgccggaaccggcggaggccgcccccgcgcgcggcccgcccaccggtgcggcctcggggccgggcggcgcctgctccagcacggcgtgcgcgttggtcccgctgatcccgaacgacgacaccgccgcgcgccggggccgcccggtccgcggccacggccgcgtcccggtcaccagctccgccgcgcccgcctcccagttgacctcggccgacgcgcgcccggcgtgcagggtcgcgggcacgaccccgtgccgcagcgccagcaccagcttgatcagcccggtgaccccggccgcgccctgggtgtgcccgaggttggacttcaccgagcccagcagcaccggctccccccggtcccgcccgtaggtcgccaggaccgcctgcgcctcgatcgggtcacccagccgggtaccggtgccgtgcgcctccaccacgtccacctcggccggggacagcccggcgtcggccagggccgcgcggatcacccgctgctgggccgggccgttcggcgcggtcaggccgttggacgcgccgtcggagttgaccgccgtgccgcgcagcacggccagcacggcgtgcccgttgcgccgggcgtcggacagccgctccagcagcacgagcccggcgccctcgccgaacgcggtgccgtcggcgtcggcggagaacgccttgcagcgcccgtccgccgcgagcccgccctggcggtcgaagtcggcgaacaggccggtggtggccatgaccgtcaccccgcccgccagcgccagcgagcactcgcccgagcgcagcgaccgcaccgccaggtgcacggccaccagcgaggacgagcacgcggtgtcgacggtgagcgcgggcccctccagcccgtaggtgtaggcgaggcggccggagacgacgctgccggagttgccggtgctcaggaacccctcggcgtccggcggcgcccccgccgcgcggctgaggtagtcctggttcatcacgcccgcgaacaccccggtgtcggtgccgcgcagcgaggtcgggtcgatcccggcgcgctcgaacagctcccaggaggtctccaggagcagccgctgctgcgggtccatcgccagcgcctcgcgcggcccgatgccgaacaacccggcgtcgaacgcggtcgccccgtccaggaacccgccccggtacggggtcgccgggtcccagccccggtcgtcgggcggcccggtggtggcgtccacgccgtcgagcagcagccgccacagcccgtccggcgaggtgaccccgccgggcagcctgcacccgatcgagacgatcgcgatcggctcgccggacgcggccctggcggcgcgctccggccggggcgcgggcggggcgcccatccgctccaccaggtgggcggcgagcgcggccggggtggggtggtcgaacacgagcgtggcgggcagccgcacgccggtggcggcggtgagccggttgcgcagctccacggcggtgagcgagtcgaacccggcctccttgaacgcccggtccgcgtcgaccggctcccccgccgggtggccgagcaccgaggcggactcggcgcggaccagggcgagcaggtcggcgggggtgcgcgcgggtcggacggcggcggctgcggggcgggcggcgcgcacgacgtcgcgcagcaccggcgggacctgcccggtcgccgtccccgccaggcccgccgcgacgaacgccggtccggcgagggcggcgtcgaacagggcgaggccctcggcctcggtcagggggcggaccccggacgcggccatgcgctcccggtcggtcccggacagggagccggtgaggccggtcgcctcggcccacaggccccaggcgatcgaggtggcgggcagcccggccgcgtggcgctcgcgggccagcccgtccaggtaggcgttggccgccgcgtagttgccctggcccggcgcgccgagcacgccggaggccgaggagaacagggcgaacagcgccaggtcggaggtcagctcgtgcaggtgggcggcggcggtggccttgggggcgaggacccggcgcacgcggtcgggggtgagcgcggtgatcgtgccgtcgtcgagcgcggcggccaggtgcacgacgccggtgagcgggtgcgcggcggggaccgcgtcgagcgcggcggcgagggcggcgcggtcggcgacgtcgacggcgcggaccaccacgcgggcgcccgcgccggtcagctccgcgaccaggtcggccgcgccgggcgcgtcggggccccggcggctgagcagcagcaggtggcgcgcgccgcgggcggtgacgaggtggcgggcgacggccgcgccgagcacgccggtgccgccggtgaccaggacggtgccctcggggtccaggacgggtgcggcgccctcgggggcggcgcggcgcaacctcggcgcggtgacccggcccgcgcgcacggcgagctggggctccggtccggcggcgagcgcggtgatcaggtcgtcgggggcggagccgtcggtgtccaccaggacgatccggccgggctcctcggcctgggcggagcggacgaggccgtgcacggcggacgtcgcggcggtcgcgccgtccgggccctcgccgcccctggtgaccacgagcagcaccgagtcggcgtgccggggatcggcgaggaacccctgcaccacggcgagggtggagcacaccgcgtcctcggcggaggccgcggggacgtcgtggcgcagcaccggcctgggcgcggtcgcggccggggtcagggcgggcgtttcggtccacaccacctcgaacagcgaccgcggcgcccgcgtggcggtcaggtcgcccgccgggcgcagcaccagggagtccacggtgaccacgggcctgccgtcgccgtcggacgcggtcagcgccagctcgcccgccctcggggtggtcagccgcacgcgcagcgcccgcgccccggtggcgtgcagcgccaccccggtccaggtgaacggcagcagcaccgggacgccctcgggcgcggtgacggccgccgcgtgcagcgccgcgtccagcagcgccgggtgcacgccgaacgcgccgggctcgcccggcagcacgacctcggcgaacacctcgccgtcgcgggtccagcagcggcgcaggccgcggaaggcgggcccgtacccgtagccggtggtggcctggcgctcgtagaacccggtcaggtcgacctcgcgcgcgccgggcggcggccactcggcgggatcctcggcggtcacgggcgccgcggcggcgaggtgcccgcgcgcgtgcctggtccagtccccgccctcccccgagtgcagctcgaccggtcggcggccggtggcgtccggcgggccgacccgcacctgcagcggcgtggtccgcggcccgagcggcagcggctgctccacgaccagctcgtccagcaccgggagcccgacctcgtcgcccgcgcggaccgcgagctccacccagcaggcgcccggcagcagcgcggtcccggacacggcgtggtcggccagccacgggtgggcgcgcgtggaggcgctaccggtgcacaccacgccgtcgtcgcccgcgagcacgacgacggtgtcgagcagcgggtggttcgccgcgcgcagacccggctggtcggtggccgcgcggcgggcgtccggccagcagcgctgccggtggaacggggtggtgggcaggtccacgcgccgccaccggtcaccggacccggcgttccagtcgacggggacgccgcgcgcgtgcagctcggcggccgagagccggaaccggtgcagcccgccgtggtcgcggcgcagcgtgccggtgacgagcgcgcgcccgtcggcgctgtcctgcaccgcgccggtgagcacggggtgcgcgctgacctcgacgaacgcccggtggcccgcgtccagcagggtcgtggtggcggcgtggaagccgaccgcgccgcgcaggttcgcgaaccagtacccggcgtccagctcggggccgtccagccaggcgccggtggtgctggacaggaacggcacgtgcgcgcgccggggccggacgggggcgagcgcggtggccaggacctcgcgcagcacgtcgacccccggcgagtgcgaggcgtagtcgaccgggacgcgccgggcccgcacgccggtcgcggtcagcgcggcgagcacggcctccacgtccgcgacggggccggacagcacgacggactccggcccgttgaccgccgcgaccgcgagcccgaacgactcgacctcgtgcggcggcagggcgacggacagcatcgcgccccgcccggacaggtgctcggccaccgcgcggctgcgcagcgccacgaccagcgcggcgtcctccagggtgagcgcgcccgcgacgcaggccgcggcgatctcgccctgggagtgcccgaggacggcgtccggttcgactccgtgcgcgcgccacagcccggcgagggacacggcgatggcgaacgcggcgacctgcacggtctcgacccggtcgagggggccgccggtgagggcggcgcgcaggtcgacgtcggtgtgcggggcgagcgcggcggcgcagcggtcgaccgagtcggcgaacaccgcggactcggtcagcagccgcgcgcccatgccgacccaggcggtgccctggccggggaagacgaacgcggtcctgccgtccacgtcggccaccccggtgaccaggtccgggtggggctcgcccgccgcgagcgcgtcgagcgcggtccgcgccccggcgcggtccgcggcgacgaccacggcgcggtgggtgagccgggcgcggccgtccgcgagggaaccggcgacgtcggcgagcgcgaccggcccgtccaggagcgcgcccacccgaccggcgagcgcgcgcagcgcgggttcggcgcgcgcggacagcggcagcggcagcgcgcggtcgtccctgggcggcaggtcgggggcggggacgccctccaggacgacgtgggcgttggtgccgctgacgccgaacgcggacacggcggcgcgccggggccgcccccggtcgggccacgggacgtgctcggtggccagcgcggcgttcccggcgctccagtcgacctgggcgctgggccggtcgacgtgcagggtgcgcgggacctcgccgcgccgcagcgcctccacgaccttgatcagcccggccgcgcccgccgcgccctgggtgtgcccgaggttggacttgaccgagcccagcagcaccggttcggcccggtcctgcccgtaggtggccaggatcgcctgcgcctcgatcgggtcacccagccgggtgccggtgccgtgcgcctccacgaggtccacgtcggacggccgcaggcccgcgacctccagcgcggagcggatcacccgctgctgggagacgccgttgggcgcggtcagcccgttggaggcgccgtcggagttgacggcgctgccgcgcagcacggccagcaccgggtgcccggcgcgctcggcgtcggacagccgctccagcagcaccaggccgacgccctcggaccaggcggtgccgtcggcggcgtcggcgaacgacttgcagcgcccgtccggggcgagcccgcgctggcgggagaactcggggaacgcgccgggcagcgacatgaccgtgacgccgcccgcgagcgcgagcgagcactcgcccgagcgcagcgcctgcgcggccaggtgcaggccgaccagcgaggaggagcagccggtgtccagggtgacggccggtccgtgcaggccgaggacgtaggcgacccggccggagatgacgctgggggcgatgccggtgcccaggtggccctccatgccctcgggcacggggtgcaggcgggtggcgaagtcgtagtagacgaccccggtgaacacgccggtccggctgccgcgcagcgaggtcgggtcgatcccggcgcgctcgacgacctcccaggcggtctccagcagcacgcgctgctgggggtccatcgccagcgcctcgcgggggctgatgccgaagaaggcggcgtcgaactcggtggcgtcgtgcaggaacccgccctcgcggacgtaggagcggccctgcgcctcggggtccccgtcgaacagggcgtcgaggtcccagccccggtcggcggggaacccggagatgacgtcgccgccgcgcgccaccaggtcccacaggtcctcgggcgaggcgacgccgccggggaaccggcaggcggtggccacgaccgcgatcggctcgcggtgcgcggtctccagcgcgcgcaggcgctgctggacccgctcgttctcggccagcgcgcgcttgaggtagtcgcgcagcttcttctcgtcggacacgtgtccctctcaggcgccggtcgggccgagcctgctgtcggccaggcggaacagctcgtcgtcgctgatgtggtcgagatcgaggtcgtcgggtccgccgggtccgccggtcagctcctcccagcggcccaggacctcgcgcaggcgcgcggtgacccgctcgcgcagcgcgtcgtcgcccgcggcggcgggcagggcggcggccagggcgtccaggtcgcgcagcgcggacgtggggtcctcggcgtccggggccgcgtcgggggcggcgctgtccctggcggcggcgaggtgctcggccaggtgctcggcgagcgcgcgcggggtggggtggtcgaagaccgcggtgggcgtgagcttcgcgccggtcgcggcgcaggcccggttgcgcagctcgacggcggcgagcgagtcgacgccgaggtcgcggaaggccacgtcggcgggcaccgactcgggtccggggtggccgagcgcggcggcggcgtgcgcgcgcaccaggtcggtgagcctgcgcagccgctcgggcgcgggcagcgcggccagctcgcccgccagcccccggtcgggctcggcggtggcggcgaccggttcggggcgggcctcgggcaggtcggcgagcagggggctgcggcgcagggcggtgaaccgggcggcgaacgcctcccagcgcacgtcggtcacgacggtggtgctctcgtcgcgggcgacggcgtcgagcagcgcggacaccgccaggtccgggggcatcaggcgcaggccagcctgggcgagctgggcggcgatgtccggggtggcgctggacatgccgccgccgtcccaggcgccccaggcgacggaggtggcgacgcggccgtgcgcgcggcggcgctgggccagtgcgtcgaggcgggcgttggcggcggcgtaggcgcactggccgccgctgccccacacgcccgcgttggaggagaacaggacgaacgcgtcgagcgggcgcccgcccagcagctcgtcgaggtgctcggcgccgagcgccttggcggcctggacgcgcgcgtgctcctcgggggtggtgtcggcgacgggggtctgggcgttggcgcccgccgcgtgcacgacggcggtgacctgctcgcccgcgcgctccaggccggtcagcagggcggcgagcgcggcgcggtcggcgacgtcgcaggcggcggtggtgacgcgggcgcccagcgcctccaggtcggcggcgagcgcgggcgcgccgggggccgaggggccgcggcggctggtgaggacgaggtggtcggcgccgagggcggcgaggcggcgggccacgtgcgcggcgacgccgccggttccgccggtgatcaggaccgtgccgcgcggggtccacggggcggccggggcctgcggttcggcgcgcacgagcctgcgcgcgaacacgccctgcgcgcggatggcgacctggtcctcacctccgtcgtcgacgccgggtccggcgaggacggcggcgagcccgcggcgctggcgcgggtcggcggggtcggcgaggtcgaccagcccgccccagcggccggggtgctccagcgcgacgacgcggcccagtccccaggtggcggcctgggcggggtccggcgggccgtcgtgcggggagatcgcgacggccccggtggtggcgcaccacagcggcgcggtggcgcccgcgtcgccgagcgcctggaccagggtgagcacgtcggcgcccggcgtggcaaggacgcccgcgacggcggtgtgcgggcggagcaggtcggcgagggcggcgcggtcggcgtcgccggggtcgagcgcggcgccgggcagcaggtcggccagcaggtcggggtgcggtccgacgacgagccagtcgccgcgcggggcggactcgggcggttcgggcagggtgcgccaggtgatccggtagcgccaggagtccagcagggcccgctcgctcctgcgctcgtgccaggcggtcagcgcgggcaggacggcgcgcagcgcggcgcgctggtcgggctcggtgagctccagctcggcgcacaggccgtcggcgtcgcgcgccgccgcccacagcgggtcgtcgccgcccgcctgccgggcgtcgggttcggcctcctggagccagtggcgggcgcgctggaacgggtaggtgggcaggtcggcgcgccgggcgtccaggccctcgaagacgggggcccagtcgacgggcacgccgcgcacgtgcagcgcggcggcggagcgcaggaaccggtcgaggccgccgtcgtcgcggcgcagcgtgccggtgaccacggcggtccgctcgcccaggacgtcctggatcgcggcggtgagcaccgggtgcgccccgacctccacgaacgccgcgcagtccgccccggcgagctcggtgaccgcgtcggcgaagcggaccggctcgcgcaggttggcgaaccagtagtcggcgtccagctcggcgccgtccagccacttccccgtgacggtggacagcatcgggatcgcggccggtcggggcgtgacccgcgcgaggtcggccagcagccgcccgcgcagcacgtcgacggcgggcgagtgcgaggcgtagtccaccggcagcctgcgcgcgccctccacccgcgccacgagcgggtcgacgtcgccggagacgaccaccgatccgggtccgttgaccgccgccacggacaccccggtgccggtgatcaggcgttcgacctccgcgcgcggcagcgggatcgaggccatgccgccgccgcccgcctcggcggcgatggcccggctgcgcagcaccaccacccgcgccgcgtcctccagcgagagcgccccggccacgcaggccgccgcgatctcgccctgcgagtggccgacgaccacgtccggctcgacgccgaccgagcgccacagccgcgccagcgccaccatcagcacgaacgacaccggctgcaccacgtccacccggccgaggtcgccgccgcgcagcgcgtccaccacgcggaagtccaccagcggctccacggccgccgcgcagcgcgccacggccgccgcgaacaccggcgactcgtcgagcagccgggcgcccatgcccacccactggccgccctggccggggaacacgagcaccgtgcggcccgccacgtcggcccgcccggtcaccgcggcggccgagggccggtcggcggccagcgcctccagcccggcggtgaacccggcccggtcggcggcgaccaccaccgcgcggtcgaccagccgggcgcggccggtgagcagcgaccagcacacgtccaccggctcccccggcagctcgaccaggtcggccgccgccgcgcgcagcgccgcgcccccgcgcgcggacagcagcagcggcagcgcgcccgccggacgccgctcccccggcgcggacggcgcgtcgccctcctccacgaccaggtgcgcgttggtcccgctcatgccgaacgcggacaccgccgcgcgccggggccgctccccgcgcggccacggcgcgggttcggtcagcacgcgcagcgcgccggaggtccagtcgacctcgggggtgagccggtcggcgtgcagggtgcgcggcgcgacgccgtggcgcagcgcctgcacgaccttgaccaggcccagcaccccggccgcgccctgggcgtgcccgatgttggacttgagcgagccgagcagcagcggcctgccctcgggcctggcgcgcccgtaggtggcctggagcgccgccgcctcgatcgggtcgccgaggcgggtgccggtgccgtgcgcctccagcaggtccacgtcggacggccgcagccccgcgtcctccagcgccgcctcgatgacgcgctgctgcgcggccccgttgggcgcggtcagcccgttggacgcgccgtcggagttgaccgcgctgccccggatcaccgccagcaccggcctcccggcgcggcgcgcgtccgagagccgctccagcagcaccacgccgacgccctcaccccacgccgtgccgtcggcgtcggcggagaacgccttgcagcgcccgtccggggacagtccgcgctgccgggcgaactccacgaacacgtccgacaggggcatgaccgtcgcgccgcccgccagcgccatcgcgcactcgccgcgccgcagcgactgcgcggccaggtggatcgcgaccagcgacgacgagcaggcggtgtccacggtgatcgcgggcccgtgcaggccgagcgcgtaggcgacccggccggaggcgacgctgccggagctgccgatggccaggtggccctcgaactcctcggcgacgcgccgccggttgccgtagttctggtagaccacgccggagaagacgccggttccggtgccgcgcagcgacagcgggtcgaggcccgcgcgctcgaccgcctcccaggaggtctccaggagcatccggtgctgcgggtcgatcgtgacggcctcgcgcgggccgatgccgaagaacccggcgtcgaactcggcggcgtcgtgcaggaacccgccctcgcgggtgtagcaggtgcccggcctggtcgggtcggggtcgtagagcccggccaggtcccagccccggtcggtggggaagccggtgatcgcgtcgacgccgccgtccaccagctcccacagcgcttccggggtgcggacgccgccgggggcgcggcaggccaggccggtgatcgcgatcggctcgtggcccgccgactccaggtcggcgagcttgcgccgggtctcgtgcaggtcggcggtgacccaccggaggtagtcgaccagcttctcgtgggtcccgtcggtcatgggtgtgcgcgtctccagggctcgggcgggtggtcgggtggtcgggcgggggtggtcaggcgggggtggtcaggtcggcgtgcagggtggtgggcaggtcggcgcggtgggtgacgcgcagcttgcggaacaccctggtcaggtgctgctcgacggtgctcggggtgacgaacagcttctcggcgatctgccggttggtgaggccggtgacggccagggcggcgaccttgcgctcggagccggtgaggccgctggtgtcgtgctcgacggggtcggccgcgtccggttccaggcgcgcgcacagcggtcgcgcgccacacgccctggccaggtgccaggccctgcgcagcacggcgcgggcgcggcggtggtcgccgagggcgtggtgcgcggcgccgaggcaggcgagggtggcggccagctcgtagcggtcaccgcagcgctccagggcgtcggcggcctcggcgagcaggtgcgggcggcgggtcggcgggctgagcggggcgagcgcgcgcagcgccagcgccctggggcggcccgcgtcggcgccggtcgtggcgagctggtcgaacaccagccgccgggcgcggtcgtggttgcccaggcgggtccacgcctcggcggccccggtgcgccacggcgcgaggccgggcaggtcgaggccccacgagcgcaggaggtcgccgcacgccaggaagtccgcgagcgccgcgtggtcccgcccggacgcgaggtggtggtggccgcgcgcgtgcaggtagtgcggggcggcggggccgtcgaagagggcgtccggcacgggcgcggtgacgtgccgcccggcctcccggcgccgcccgcgcagggtgtcggcgaggatcagcgagcccagcggcaggcccacggacgcgccccagccgtgcgaggggacggcgtcgagcgcggcgcgggcgtgcccggcggcgcccgcgaggtcgccgagccgcagcgcgatcaccgaggcggcggcggcgaaggaggcgacccaggtgggcgcgcgccgggcgcgggcctcgtcgagcagccgggcgcaggcccggtcggcgtcggcggcgcggtcggcgcgcaccagggctagcagggcgagcagcgcgggttcctcggaccacggggaggtgtggctgaggtgggcgtggcgcaggtagtgcgcggcctcggcggcggcccggcgctggtccccgagcgcgaacccgccgaccaggctggcggcctcgcgcaaccacgggtcgacgcggctcgggggtggggcgaggggggtggggtgggccgaggtgcgcggggcggaggtgtgcggggcggaggcgtggagggttgtggggcggcgggaggcggtgagcggcgggcaggcgagggagagccaggtctccaggtcgggcagctcggcggcgtgctcgcgggcgcgcagcgcggcgagcacgcgcccggcggcggtggtgcgcccgtgccagaccagctggcgcaccagcgcggcggcgtcccgcggggcgagcgcgtcggggtcggcggcgagcgcgtcggcgagcgggtcgaggtggcacagcgcggcagccgggttgagctgccagcgcgcgcgggcgagccgggcggtgagcgcgggcccggcgacggcggcggcctcgggcgacgtgcggcgctcggcgggcgcggccgtccggtccgcgcacccgcaccggtgggcggcgacgggcgggtccgggactgcgcgcggggggacggcccgcggttggtccgcgtcgtcccggcggtgggcggcggcgagccgtgcgacggcggtgggcacgtcgtcggcggcgagcgcgtggtcggcggcggcgacgagcacgtcgacggcccaggcgggcgcgggcagcccggcggtgacgaggtggtcggcgacgacggcgggcgactcaccgcgctcgtgcaacgtccgcgcggcgcgcaggtgcagctcggcgcgccgggcggcgggcagcacgtccagcacagcggcgggatcggcccggtcgagcccggctcccgcgaggcgggcgcgggcgtcgacgacggaggcgggggcccgaccgagcaggtcggcgaccaggtcgacgggcgcggcggggtccgaggacgcgcgggggcggaccccagcgggaacgacgtgatccgcagccacccgaccgaacgcggaagccccgtcccccccggcgggatcagccctcaccccagacaccgggccgggcacgagcggctcagcgccccccggcgaggcaccggcgcgaccgggcgcgaccggccccactcccccctgccggacgcccacctcgccagacgtgagcggtcgcgccccgccgtcgggaagcgccccgccagggggcgcggccccagggggcgcgtcggtgggaagcgcggtgccagggcacgcctcgccgggacgcacggcggcggggagggcccagtctggaagcgcggcggcggaacgcacggcggcggggaagggcccgccgagaacgtcggcggcggggaacgccccgccgggagacgcgacccccggaagcaccccgtcgggaagcgcggtgcctgggcacgcctcgccagggcgcacagcagcggaaagagcctcgccagcaggcgcggtgccttcgcacgcctcaccagggcccgcagcagggtgcgccccgtcaggaagcgcggcaccaggaagcaccccgccgggaagcacggcggcgggtgcggtgagaagcgcggcggtgggtgcggcgcgaagcccggcgggaagcgcggcggcgggtgcggtgagaagcccaacggcgggcccggtgagcacggcggtggcgtgcgcgacggcggccacctcggcgccgcagcggtcgagcaccccggcgagggcggcgcggtaggcggcccccggtcccgcctcccccgccacgaggtcgtgggccagcccgcgcaccagcgcggggttccccccggtcaccggccacggggacccgccgagcagcgcggcggtggtctccggtcgcagtggcgggaggtcgagcacgagggcgcccggcgtgcgcgccacggcgcccagcgggtcgtcgtcgggcaggcggggcaggtcggtgagcgcgaggacggcggtcgtgcgtctcgcccacgccaccaggtcgaacagcgcgcccacgtcggcgcccccggcgtcgtccacggccaccagcagcgcgccgccgcccgccgcgacctccgcgacctcgggcagcgcgtccgcctcgaccaccaggcaccggaaccccgcgccctcggccatcacgaccaggcgccccagcagcgcggtcttcccgtgccccggcgcgcccctgagcagcaccgcgcccccgcacccgcccgccgcgccctccagcagggcagccagcgcctcgcactgcccgtcgcggtccaccagccgcatccctcgcctcccccgcgtccgccccgccgaccttcaggcatgtcaacagccctgtcagtcgtcttgtccacgccccgaccaccgcacccccactggccgcccacccgcgccgggaaaaccacgaagtcccctgccgcgcgggcgtcccaggtcggacactcctgagcgcacccccgcacccgttccaggaggcgaccaccgtgacgttgaccgacgacgagcacaccgcgcaccggcacgacgagggcgccgcggccttccaggcgctcgcgaccgccgccgcaggctggggcgcgctgcgcgccgccgtggagctgcgcctgcccgacctgctcccggacgcccccaccgccgtccccgacctggcggcgctggcgggcgtcgcgccgacgcggctcgcgcagctggtcgcggtgctcgccgacgtgggcgtgctcgcgctggacgagcgcgcccacgtccgccacacgccgctctcccgcgccctgcgctcgaccgacggcctggcgctcacccgcctgctcacggcgggctgggcggacgaggcgtggcgcgggctcgcggagggcgtgcgcaccgacacctccccgctggagctggtgcacggccgcccgctgttcgacctcctcgccgccgacccggccgccgccgcggtgttccacgacgcggtggccaccggccccggcgacccgttcgacgaggcgatccgctccgcgctcgacctgaccggggccgcgcgggtggtcgacgtcggcggcgggcggggcaggctgctcgccgcgctgctgcgcgagcaccccgtgctctcgggcgtgctgttcgacctgcccgaggcggtccgggacgcggttcccgagctgcgcggcgacgaccgggtccagatcagggcgggcgacctgttcgcggaccgggtcggcgcggcggacgtctacgtgctgcgcacggtcctgcacgtgctggacgacgcgcaggccgtgcgggcgctggccaacgtccgcgccgacgcgcccgcgcacgccagggtcgtggtcgtggaagcgctttccgacgacgaggccaaccggacctacgcggcgcacagcagcctgcggctgttcgtgctgtgcggcgggcgcgagcgctcgacggcggagttcgccgagctgttcgaccgggccgggctggagctgcgctcggtcaccccgaccgggacgccgtaccacctgctggagggcgccccacgcctgagcccgcgcacggacgggtgatcgtcagctctcaccgggccgcactggtgctgctctgctcgactcggtccgcgctgaccgccgccccacctgacctgttcgccggaccgttcgcgggatccgccgccgcgggtggtcgggcgcccctaccggccgcacgacagccgcagggcgcggagcacgatcccggtggtcggcgtggtccgcgccccggtggcgggacccgcgttccgagcgacgtcgagcgagagcgggtcgacgacggtcgccatcgcggacgcgggcgccgagaactgcctgaccgcgctggagtcggtcgccttcgagggcaggcgcctggccggcgaggacgtctggtgcgtcgcgttcgtgagcccggcgcggttcccgctgaccaaccagttcggtctggcggtggtcgggaccggcaagcccgccaagtcgctgtggacagggggtggactaccggagtccttcgagcgcggcgacctcccgcccggaccgccgggcgatcagcgtcagcagcgggccggtcgccgccgtcgtcaccagcgccatcacgaccaggacggtgaacaggcgcccgtccagcacccccagctccagccccacgcccagcaccaccagctcggtgagccccctggcgttcatcagcacgccgacctcggccgcgtcgcgcgcgggcagccccgcgagcagaccgccgcccgccgcgccgaccagcttgccgacgcacgcggcgacgagcaccagcagcgcctccgccgcgccggaccagccgaggccgccgacgtccacggacagcccggccacgacgaagaacaccggcagcagcagggagctgagcccgccgacccggtccaccagggtcggcgcggcctcccggagcgcgtccttgggcacaaccgccccgaacaggaacgccccgaacaccgcgtgcaggccgatggccgtggtggcgagcgcgaacagcagcagcccctggacgacgaccgcgaacagcaccgcgtccgcgcgcggcgagccgcccaccgtccgggcaacccaccccaccagcggccggacgaggagcaccgccaccaggaccagcgccgccgtccaggccagcgtggccaccgccgaccaggggccgcgcgcggtcacgatcccggcgacgacggcgagcgcgcaccacgccaccacgtcgttcaccgcggcgctcgccagcgcgaccaccccggaccgggtgcgctgcatccgccgctcggccaggatccgcgccagcacggggaaagcggtgatcgacagggcggctgcggtgaacagcgcgagcgcgtcaccgccctgcggccacagcagcgccgcgacgcccagcccgaggcagaacggcagcacgaccgagcccgccgacaccgcgccgaccacccgccccgcgccgcgcagctccgcgacgtccagctcgcagccgaccaggaacatgaacagcaccaggccgagctgggcggcgaacgccagcggcgggcgcgcctcgggcgggaacagcagcgccaccgggtcgccgggcagcaggccgagcaggctggggccgagcgcgatcccggccgcgacctcccccaccaccgagggctgccccagccgcctggccagcgcgccgagcagggcgccgagcgccaggacgaccgcgaggtcgaggaacagcagcgcgacgagcgagtcgcccgccggggccgccaccggcgcgctcagcccctcgcccccgcctggagctcccacagcgaggcgtagcgcccgccggaggccaccaggccctcgtgcgcgccctgctcgacgacgccgccccgctccagcacgtagatgtggtccgcgttgcggacggtggagagccggtgcgcgatcacgatcagggtgcggccccgcgcgaacgtcgccagcgcgcgctggatcgccgcctcggtctcgttgtccaccgccgacgtggcctcgtccaggaccaccacgggcgggttcttcagcacggtcctggcgagcgcgatccgctgccgctgcccgccggacagcgccgcgccgtgctcgccgaccaccgtgtcgtagcccaggggcagcgactcgacgaacgcgtgggcctcggccgtgcgggcggcctcgcggacctgctcgtcggtggcggagaacgtgccgtagcggatgttgtccgcgatcgtgccgtcgaacaggaacgggtcctgggcgacgaacccgacggccgcgcgcaggtcgggcaggcgcaggccgcgcacgtcccgcccgtcgagcagcacccggccggtgtccgcgtcctggaagcgcatcagcagcttggcgacggtggtcttgcccgagccggtcgcgccgacgatgccggtgacccggcccgccgccgcgcgcagcgacaaccggtccagcacgcgcgggcggccctcgtaggagaaggacacgcggtcgagcgcgatggcgcccgcgacccgctcgcgggccagcgcgcggccaccggtgggctcggtgggcagggacatcaggtgctcgacccggtcgtgcgcggcgagggtgcgctggtactggtcgacgatcgggcccagccgggtcagccgcatgaccatctgctgcggcagcccgatcagcgggctgaaccgctcgaacgggagcgtgccgtcgagcaccttgagcccgcccacgagcagggtgcccgccatggagcaggtggtccaggcgcggatgccctcgccgaaccggacggcgtgctgctgggtgcgcgtggccgcgtcggcgcccgccagctccagcaggtcgaccagctcggcctcgtggtcctcggcgcaggcgctcttgaccgtggcggcggcctccagtgcggcggagagcctgctgcgcagcagggcggcctgctcgcccgcgctggcgtagtcggcggcggagcggtcgcgctggcgcagggacagggccgcgacgaacggcaggggcaggaacgcgatccaggcgatgccgggcacgagcaccaggaacgacgggaccaggatcagcgcgctggtggcgagctggaccgcctcgcccgccgggccggagaagaacgcgccgagctggtcgacgtcgtcggcgagcacgcccgcgacgcggctgggccgctcgcgctggatgtgccgcatctccaggcgctgggtgtggcggtgcaggcgggcgcgctggtcgcgccggacgccctggccgagcgcgcgccagcgggcgccggaggcggcggagagcacgacgacgaccccgcaggtcgcggcggccccggcggcgagcgcgagcagctgggcgggggcgctcgtgacgcccagcgcggtgagcggggcgtactcgcccttgatcagcaccagcaggatccagccgaggaacagcccgaggccgagctcggcgagctggcaggccaccgtcagcgcggcggcggcggcgagcgccgaccggcggcggccgagggcgcgcaccagcgggtgggcggagagccgggtgcccgcggcggtgcggcgcaggggccgcaggagggcgcgccaggcgtggcgcaggacggcggacgagacgccggtgaccacgccgaccagggcgagcgggcggcgcaggacggcgctcgcgggccggagcgcgacctcggacgtgccgccggacgtgctcgcggcggtggccggtgggctcgtcgtggtgctgccctcgacgaccagcggggcgctggccgcggcggcgaccgcgtcccgcagggcggcgccgacctcggcgacgccgagcgcccggtcgtgcagcaccagcacccggccggtcgcggggctggcctgcgccccggtgatgccgggctggcggagcagcgcggcttcgagggcgcgcgcggcgcgcgggttcctgcgcacgcgcgggacgtcccagcgctggcggccggggacgaccgagcgcacccggaacccggtgagcctggtggtcggggcggcgtcgaggagctgggtcacggtggggccttctctccgggcgggggctcggggtcgggggtcagcgcgctctggcgtccggcccggtcccggcgggcgcgcccctcgggggctccaccacgtcggcggcgagccgggcggcctccagctcggcggtcacctcggcggtgatgtcctggagctcctcgccgacggcgtgcgcggcgcggcgggcaccggcgacgatgcccgcggaggtccggacgacgccgcgggcgagcggcttggcgaccctcctggccacggggacggtcaggagaccgagcaggagaggagcgacgaagggcatggtgctccccgatccctcaggcggtggacgcgcgaagacgatcagcgcttgggcagcacgctagtggagtgggcgggtgatcccgcccgatcgaccgggggttcacactttccggtggacggcgcgcgcgggcggggcgcggtgggcctcccgccgcgcggggcggttcagcggtcctgcggcgcgcccgcgctgaggtcggcgtggggatcggcgtgggggtcggcgtgggggtcggcgctgaggtcggcggcgtcccaggcggcctggatcatccggaagacctcgtccacggccgcctcggggtcctgggcctcgcgcgcgaggacgtgggcgtcgaccgcgaagcgggcgatcgcccggcaggccgtggtgcgcggcggcaggccgagttcggcggcgatggcggtcgcgagggaggcggcgtggcgggcgcacatggcctcgttgtagccgcgcaggtccggtgaggactggatcaactcccccagcgcggccgacctgtcggtggtgcagtgccgcacgatcgcgtgaacctcgtggcgcagtgcggggacgaggtcgtcccgcccgcgctcggcgacggcccggacgaggcggtgctcgaagtcggcgtcgcgctcgaagaccagggcctccttgctgtcgaagtgcgcgaagaccgtggtgacggcgacgtcggcctcggccgcgacgtcgcggacgccgaccgcgtcgtagccgcgctccaggaacaggcgcagggcggtgtcggcgatcttctgacgggtcgcggccttcttgcgctcgcgacggcccggtggcgcggtcacggcgggatgctatcaggtgttaaatcgtagctgttccaaaaccctaaccgttagggttagtgtcgggggcatgaagaagatcagctacagcgagcacggcggtccggacgtgctgcgcctggtggacgccgacgagccgagcgccgggccggggcaggtgcggatcgcggtgcgcgcggcgggggtgaacccgttcgactggcgggtgcgcgaggggcagttcccggtgccgctgcccgccgggaccgggcgggacgcggccggggtggtggaccgggtcggcgcgggggtcgacggggtcgcggtcggcgaccgggtgttcggcgaggggatcgagacctgcgcggagttcgcggtgctgacggcgtgggcccacctccccgagggcctctcgttcgaggaggcggccgggtacccgtcggtggtggagacggcgctgcgggtgctgcgtgaggtcggggtgcgggaggggcagtcgctgctggtcagcggcgcgtccgggggtgtcgggtcggcggtgctccagatcgcgcgcgaccggggcgtgcgggtggtcgggaccgcgagcgcggcgaaccaggactacctgcgcgacctgggcgcgaccgcgacgacctacgggccggggtgggtcgagcgggtgcgggcgctggtcccggtggacgcggcgctcgacctggccggggcgggggtgctccccgacctggtcgcgctgaccggcgacccgcggaaggtgatcaccatcgccgaccgggccgccgccgagcacggcgtccggttctccggcgcggcgcacgacgtgcccgccgcgctggccgaggcggtcgacctgatcacgaggggcgccctgcgcatcccggtcgaacgcgcctacccgctcgccgagacggcgcgggcccacgccgacagccaggccgggcacgcgcgggggcgacgggtgatcgtgatctgagcggggccgccgacgaccgggctgagcggggggcggggggcggggggcggcgggcatccgctcggtggccgccgccggcgcgcgcgcgggcga

SEQ ID NO: 57 - DNA, 11905 bp, Actinosynnema pretiosum
gcgcgccgtcagggacgcgttcgcgccgctgtcgcccggcccggtccgggcgtgtgctggcgggcatgacactcgcggagttgctgcccagcgtcggcatgaccggcgaaccggccctggagacggggctgtggcccctcgggacccggctggagcggggggagctgctgctcggcggggtgcccgcgaccgagctcgccgcccggttcggcacgccctgccaggtgctcgacgagggcacggtccgggcgcgggcgcgcgcgttccgggaggcgctgccggaggcggaggtggtgttcgcgggcaaggcgctgccgtgccgcgaggtgtaccggtgggtggcggacgaggggctgtcgctggacgtgtgctcggccggggagctggcgatcgcgcggtcggtcgggttcccggcggagcgcgtcctgctgcacggcaacgtcaagacccctgaggacctcaaggccgcgctcgggtacggggtcgggcgggtggtggtggactcgttcgacgagatcgagcagctcggggcgctggccgaggggccgcaggacgtgctggtccgggtcacccccggcgtcgacccgcgcacccaccgggcggtggcgaccggggtggaggaccagaagttcggcttctcgctcgcgggcggggacgcgctggaggcggtgctgcgggtggtcgagcagccgtcgctgcgcctggtcgggctgcactgccacgtcgggtcgcaggtgcggcacgtcgcggtgtacgaggaggctgcgcggcgggtggtcgggctgatcgcctcgtgcggggtgcggatcgagcagctggacctcggcggcgggttcgcggtgccgtacctgccgggggagggggagttcgacctgggcgggttcgcgcaccgggtgcgggtggcgctgagccacgagtgcgcgctgcggcgcgtgccggtgccgaggttgtcgatcgagccggggcgggcggtcgtggcgcgggcgggcgtgacgctgtaccgggtggcggcggtcaagcgcggggtgaggcgggtgttcgtggccgtggacggcggcatgagcgacaaccccaggcccgcgctgtacgggagccggtacgcggtgcgcctggtccgccgcggcgggcggcgcgcgccggtgacggtggtgggcaggcactgcgaggcgggcgacgtgctcgccgaggacgtgccgctgcccgaggacgtgcgcgccggggacctgctggcggtgccggtgaccggtgcttaccaccacgcgctggcgtcgaactacaacgccgtgggcaggccgccggtggtgggggtgcgcgacggcgtcgcgcgggtgctggtgcggcgggagacggaggaggacctgctgcggcgggaggtgtgaggggtcggtcctgcctgcgcctgcgccggtcttgaggttgccgtcccgctgcggcccgagtgctcgtgagcgctcgggccgcggcgtcggggggcggcgctcagggcgcggggacgttcggcagctccgcccacacggtcttgccgccggggtggggcgcgctgccccagcggtcggtcagctggcggatcagcagcaggccgcgcccgcgcgtgccgatctggcgcaggcccgcggtgaggtccgggcgcggcgggtcgggggagccgtcctcgacctccacgcgcacgtggtccggggtggtcgtgacgcgcagggcgagtggggaggtggtgtggtcgtaggcgttggcgaccagctcgttgaccacgagcagcacgtcgtccgccgactcctcgtccgcgtcggccaggagcgcgccggtccagcggcgcacctcggccagggccgggcgggccgtgaggtccagcgtggccggtgccggtgtgccgggctcgggggcgggctgggtggtcgggtcgtccacctcgtccatgatggccgcctttcccgcgctgtggtcggtgctcgccggggccgcggccccgacgagcgccagcgtcagggcggtggcggtggcgacacctgcgcccaggggcgggggtgtcgccgtggccgcgccctggggcgggggtggagtcgtgaccgggtctgggggcggggcctggggtggagccgttgccggggtcgggggcgtcgccagtgcctgggtcgggtccaccggcgtggtgggagcgcccacgccgaccggctccgcagcgcgcggaccgggatcgcgtcttcgacctgctgtgatctcgtccccctgggcgccgtgctggtggtccgcgggcgggtcgcccgcggaccgagggtggtcaggccgtgctgggcatcctggtcaggtcggccccgcgctgggcgtcgacggccaggtccacggtggggtgcgtggggagcagcgcgctcatcccggtcagctgcagcggccggagcacgccgcgctgcgcggtggccagcaccagccgcgcccggccgcgccgagccgcctcgaccagcagcgacaaccccgtggaggccatgaaccgcaccccggtcaggtcgagcacccccggcccgcgcaacccgtccaaccagggcagcagcgcccggcggacctcgtccgcgacgttggcgtcgatctccccggcgacgcgcagcacgggcaccccgcgcaccacgtcggaagccaccgtgaccaccgcggctgcatccagctcggacaccggcccgttcctctcgtcctgttgctgtgactgcggtgggctggctgttcaacccgctcgggatcgacgtgacgaccccggtgagcagcctagtcgctgccgggggccggcgcgtcgcggtggtcgtggtgatcgggcgcgacgcgcgagcgccgggccgtggcatggtggcaggagcgcaccggacgggcggtcgacccggcgggcccgcggtgcgcggacctgaccggggcggaccggtggcgggtcgcctgccagggtcgccgtggtcgggtcgtcggcggagagcggagaggtggcgtggtgcgcagtcccgtgcgcaggagagcggtcgtggtggtgtccggggtcgtggtggtggccgcgctcgtggggttgtgggtgttccagccgtggcggttgtggacgcgcagcgaggcggacgaggcgctgcccgccgagttcaccgcctcgaccagcgcggggccgagcaccggggcggcgagcagcggcgccccgacgcccagcgccccgacgtcggcgcccgtcgaccagcccgcggtgctggccacgggcgggttcgtcacccaggagcacgagacgcggggcaccgccaccgtgctgcgcaacggggccgcgcgcgtgctgcggctggaggggttctccacctcggacgggcccgacctgcacatctggctctccgacgcgaccgcgggcggcgagtggggcaagtacgacgacgggcgctacctgccgctcggccccatgaaggccaccgacggcaaccagaactacccgatcccggaggacgcggacctgtcggggctgcgcagcgtcgtggtgtggtgcgaccggttcaacgtggccttcgggtcggcgccgctggacctgtgacggcgtccgggcgcgctcagacgccgtaggccagcagcgggaagacggacagcgcggtgcaggcggccagggcgttgagggcgcggtactggaccaggagcgcggcgaactcgcggaggaaggcgttcgggacgaagtaccaggcggtgcgaccgccccggacgtccgccttcaggcccgcgcggcgggacaggatcgcggctcgggggacgtggtagctgctggtgaccacgaggagcctgccggtgacgccgcgctcggcgagcagggcggaggagaggcggaggttctcccaggtggaggtggactcgcgctcctccaggagcgcctcgtccgggatgccgcgctcgcgcaggtagttcgccatgacctcggcctcggaggcgggctcgtcggggccctggccgccggaggcgaccaccaggggggaagcgcccttggccgcggcgcggcggtagagcttcagcgcgcggtccaggcggcccgcgaggagcggggtgacgcggcctccggggacgcccgcgcccaggacgacgatggcggtgtggccggtgcgcttgcggacgcggccgtagagcactgagtagagcaggaagagggtgaaggcgaagccgaggtagcccgcgagcaggaacagcgaaccggccgccgccagcacccacggctcgggggagccctcgcgcagcgcggacagcaggaccaggagcaggccgcccaccacgcccaggatggccgtgcccgcgccgaaggagagggcgttgcccggcttgaaaccctcgcgccgcagcagggtcacgccgttggcgatcaggaagagcgcgatcacgacgacgaacaccggggcgatcagcaccacggcgccgaccaccagcaccctggtctgcggttcggccagggtgagcagccagagcgcgaggaacgccaggctcatcagcaggtacacgccgttgcccagtcgtctgggctcccgcttgagctgcaccaggaagacgacgagcagcaccaccgcgacgatcagagcccccatggggtgatcgtagtggtgttcgcggggtgctccgaccacaagatcgagcactgaggggtgaggggccgcgagcgcgcggcagctcgcccgcgccggacgcctttccccgaaagcgtcaccggggcgtgcggacaccccggttcccgttcatccaattgtgcgcacgaccgcgccgccgtgcccgcgtgctgcggtgatggcctgggaacaggggggattagggggaggtcaagggggttggtcgggatctgcggaatcggtagcgtcgaattccgaaagccggccgcggtgaattcccccgcgccatttccgcgaacgacgggacaggggctcatgggcagttctccggacgcaggaatcgacttcccggcctggccgcagcacgacgacgccgagcgcgcggccctgctgcgcgcgctggaccagggccagtggtggcgggtcggcggcagcgaggtcgacgagttcgagcgcgagttcgccgagtaccacggcgcgggccacgcgctcgccgtcaccaacggcacgcacgcgctggagctggccctccaggtgctcgacgtgggccccggcacggaggtgatcgtgcccgccttcacgttcatctcgtcctcgcaggccgtgcagcgcctcggcgcggtcgccgtgcccgtggacgtcgaccccgacacctactgcctggacgtcgccgccgccgaggacgccgtcacctcccgcacctcggcgatcatgcccgtgcacatggcgggccagttcgccgacatggaccggctcgacaagctgtccgcgagcaccggcgtgccggtcgtgcaggacgccgcccacgcgcacggcgcccactggcgcggcaagcgggtcggcgagctcgggtcgatcgccacgttcagcttccagaacggcaagctgatgacggcgggcgagggcggggccgtcctgttcgccgaccaggcccagtgggagaaggcgttcgtcctgcacagctgcggcaggcccaagggcgaccgcggctacttccacctcacctcgggctcgaacttccggatgaacgagttctccgccgccgtgctgcgcgcccagctcggcaggctggactcgcagatcgcgaccaggcaggcccgctggccggtgctgtccgcgctgctggcggggatcgacggcgtcgtgccgcagaccgtcgacccgcgcagcgaccgcaacccgagctacatggcgatgttccggatgcccggcgtcaccgaggagcggcgcaacgcggtggtggacgagctggtgcggcgcgggatcccggccttcatggcgttccgggcggtctaccgcacccaggcgttctgggagaccggcgcgcccgacctgacccccgaggagctggccgcgcggtgcccggtgtcggaggagatcacccgcgactgcgtgtggctgcaccaccgggtgctgctgggcgccgaggagcaggtgcgccggctggccgcggtcgtggccgacgtggtcgcgggcgcgtgagcgcggcggtgcgggttccggggacgggggaggtggcgtgagcgccgggagcggtggcgtgcgcggtggaccggtgcgggtggcggtggtcgggctcgggtgggtggcgcgggaggtgtggctgccccggttgctgggcggcgccgcctaccgggtggtcgccgtggtcgagccgtccgcgcgggcccgcgccgccgtgctcggcgacctcaccggggtcgccgcgctggagcgggcggaggacctgcgccgcgacgaggtcgacctggcggtggtcgccgtgccgaaccacctgcacgcgccggtcgcgtccggtctgctgcggcggggcgtcccggtgttcctggagaagccgctgtgcctggggtccgccgaggccgccgcgctcaccgccgccgagcgggccggtggcgcggtgctgctgggcgggagcgccgcgcggcaccgggccgacgtgcgggcgctgcgggaggtggccgcgtcgctgggcgcgctgcggcacgtcgacgtgtcctgggtgcggtccaggggcatcccggaccggggcggctggttcaccgaccgcacccgctccggcggcggcgcgctggtcgacctcggctggcacctgctcgacaccgcccgcgtgctgctgcgcggcgccaccggtgacccggagggcgcggagctggagcacgtcaccggcgcggtgtccgcggacttcgtcgacgaccacgcgttccgcgccgcctggcgcggcgacggggcgcaggcggacccggtggtcggcgacgtggaggacaccgccaggggattcctcgtcacccggtcggggatctcgatcggggtgcgcgcggcctgggcctcgcaccaggcgctggacaccaccgtcgtgcgcgtggagggcagcggcggcacggccgagctgcgctgcaccttcggcttcagcccgaaccgcgagggcccgtcgcggctcctgctcaccaccgacggccgcaccaccccggtcgagctgcccgccgagccggtgggcgccgagtacgacgcccagctcgcgagcctgcccgcccgcctggccgaccccgccgcgcgcggcgaggccgcgtccggggcgcgctggatcgcgggcgcgatcgaacgggtctaccgcgccgccgacgccgtgcggtgcgccgaacgacgacacgcgccggtcgacctcgccccgacgggagccccgtgatgaccaccggacagaccgatccccgagtcgtgcgggcccgagccgtggacgcgcgggccgtggacgcgcgagccgcgaccgccgcccccacgaccgccgcccccacgaccgccgctccggcgaccactgctccggcgaccgccgctccggcgaccgccgctccggcgaccgccgctccggcgaccgccgcccccacgaccactcctccgcgcccccgcgtcccccaggcccccgcgccccaccggcacgttccgggcgcgacctacgaccgggccgtgctgttcgacctcgacggggtgctggtcaacagcttcgccgtcatgcggcaggcgttcgagatcgcctacaccgaggtcgtcggcgacgggcccgcgccgttcgaggagtacaaccggcacctggggcggtacttcccggacatcatgcggatcatgggcctgccgctggaggtggaggggccgttcgtccgcgagagctaccggctcgcgggtgaggtcgaggtgttcgagggcgcgccggagctgctggcggacctgcggcagcacggcttcggcaccgccgtggtcaccggcaagagcggcccgcgcgcccggtcgctgctgaccaccctcggcctggcggagctgttcgaccacgtcatcggctccgacgaggtcgtgaaccccaagcccgcgccggacatgctcctgctggccaccggcctgctcggcgtcccggccgaccgggtggtgatggtgggcgacgccctgaccgacctggcgagcgcccgcgccgcgggctacccggcgctggccgcgctgtggggcgagacggacgaggcggagctgctggcggcgaacccggacgcggcggtccgcaagccctcgcaggtgctcgactggtgcctggcccacctcgccgacgaccggacgtgacgcgcgggcaggccccgttcccgccgaggcggaagtggtggtggacatgacgcggaccggttccgcgcaagcgcccctcggtggccccccggcggcgggcgccccgtcctggtccggctggtcgctggccggtggtggcgacgcccgcccggtggccgaggtgctgccccccgtcgtgcgggagctgcgcgcgcgggaccggggcgcgcactggtcgttcctgcgcgggacgggggtggcgctgtggctgggcacgaccgaggccgcgcggggcccggtgctcgccgtgctgcgcgccgaggcggcggcccgcgagctgcgcgtcgacccggcgggcgagggcttgccgggcggtccggtcgagccgctggtcgaggtcgcgacggcctccagcgaactggcgctggacgtcgcggcggcggtgggcccggcgtggggcgaggaccccggcgggtcgggcgcgcaggccacggtgcggcgccacctggccgaactggtcgcgctggccccggaaccggccaggcccgcgttcctgttccactgctggcagagctggggcagaccgctcagcccggccgcgcgcaccgaggccgccgtcgccgcgcagggcccgtcgcccggcgggcgcgtggtcggctcggccggcgcgtggcggcgctacctggaacagatcgatcgcgcgcagctgcgcaccgcggcggaccgccgccgcccggccctgccggtgctgttcgagcaggcgaccctgacccacaaccgcctcggcgtcggcgcggccgtggcggcgcgggcggcgctcgccctgcgcaccgcgctcgtgacgggcgccgcctgcggctgagggcccgcgccctcgctccgccgcccgccccaccacccgtcccgccctcccgaggaggaagcagtgcccaccaccgccaccacccgcgaggtccgggtcgagctgggcgagcgcgcctacaccgtccacatcggacacggcgtgcgcgccgccctcccggacgtcgtgcgggccctcggcgcgcgccgcgccgtcgtggtcaccgcccgcccgccggaccagacgcccgaccccggcgtcccctccctcgtcctcccggcccgcgacggcgagcacgacaagaccctcgccaccgtcgcccacctgtgcagccggttcgccgagttcggcctgacccgcgccgacgtcgtcgtctcctgcggcggcggcaccaccaccgacgcggtcggcctcgccgcctcgctctaccaccggggcacaccggtcgtgcacgtgccgacctcgctgctcgcccaggtcgacgcgagcgtcggcggcaagacggcggtcaacctccccgagggcaagaacctcgtcggctcctactggcagcccgccgccgtgctgtgcgacctcgacctgctcgccaccctgcccgagcgcgagctgcgcaacggcctcggcgagatcgcccgctgccacttcatcggcgcgccgggccttgaccggttgccgctgctggaccaggtggcggcgagcgtcgccctcaaggcccgcgtggtggccgccgacgagcgcgaccgcggcctgcggcacaccctcaactacggccacaccctcggccacgccctggagctcgccaccggtttcgcggtgcggcacggcgagggcgtcgcgatcggcacggtcttcgccggccgcctggcgggcgcgctcggccggatcggcccggaccgggtggccgagcaccacgccgtggtcgcgggctacgggctgccggtcgcgctgccgccgggcgtgccggacgacgagctgctgcggttcgtgcgccgcgacaagaagtccaccggcggcctggcgttcgtgctcgacggcccgcgcggcgcggagctggtgcgcgacgtcgacgagcacgtcgtcgccgccaccctggccgcgatgccgcgggcctgagaggtcctagcggaatcgctgggcgtgtcggtggcaggtgatgcgggcggcggggccgaggagggcttcgcggatgtcgtcgcggcgttcccggcggacgcgtagccggttcacgccgcggtgccaggcgatcggtgcttgtcgtggtcgtagccgcggtcgactcacccctctgcgcgaccgaacacgatccagatcgggcaggggcggccccgcaccccctcggggcgcccctgcgcgtcaccccgaccggtcggcgctcagcgcgggcggcagctgctcccgctgcttgatcccgagcttgcggaacacccgcgtcaggtgctgctccaccgtgctgtgcgtgacgtacagcttcaccgcgatctcccggttcgtcagccccgacgccgcgagcgcggccacccggtgctccgagctggtcaaccggcccgtgtccgacccgcaggccgccgacgcctgctgcgcgggcaccagcccgtcggcggtgggcatcagctcctcgcacagcggcgccgccccgcacatccgggcgacgtgccacccgcgccggaccacccggcgcgccagcgcgtgctcgcccagcgcccgctgcgcccctcccaggtcgcgcagggtccgcgccagctcgtacttgtcgtccacggcctcgaacaccgccaccgcctcccgcagcagccgcgggcgccgcttcaccgggccggtcgccgcgagcagccgcagcgcctgcccgcgcgcccgcctgctgtccgagcccggcctgccgagctgctcgtgcgccagcgcccgcgcccggtccgggtcgccctgcgccagccacgcctccgccgccgcggtccgccacggcaccggcgcgcacccgccgcccagaccccaccgggtcagctgctccccgcacagcaggaagtcgcccagcgccgcgcgcggccgtccccgcgccaggaggaagtgcccgcgcgcgtgcaggtagtccacgctggtgcggtgcccgaacaccccgttcggcaccggccgcgccaccacggccgccgcctcgtcgcagcggcccatcctggtcagcgccaggatccgggtgctcagcggcagccccaccgacaccccccacgcggtggggggcagcacggtcagcgcctcccgcgccagctcgtccgccctcgggtggtcgccccggcgcagcgccgactcggccgccagcgccagcacctgcgcgcgcggtaccggcagcagcggcacggcccgcgccgacagcagctgctcgcaccaggaggcggccacgtccgcgcggtccgagtacagcagcgcggacagcgcaggcacggccgcaccgccggagccgccggggtcccgcgccagccgcacctcccgcagcaccgcctccgcccggtcggccacgtggtcgccgtccccggccaccagcacgtccagcagcaccgcgttcgccctcggcgcctgctccgccctggtgcgcgcggagtccacgtcccgccccggcgcggtccccggcctggccgcaccggggtaggcgccagccagccagtgctccagcaccggcacctcctcggcggcgcgcgggtcggcccgcagcccggcgagcaccgcgtccgagtcggcggtgctcccggcccagcgcaggtgctccaccagcgtcgcgctgtcgcgcaccgacaacctgcccgcgcggaacgccgccagcagcgcggtgtggtgcctggccgaggtcgacgggttcaccagccactcggccgccgccagccgcgcctgcacggccgccctgcgcacccggtccgggctgcag